(19) United States
(12) Patent Application Publication
(10) Pub. No.: US 2022/0333198 A1
(43) Pub. Date: Oct. 20, 2022

(54) Predicting Chronic Allograft Injury Through Age-Related DNA Methylation

(71) Applicants: VIB VZW, Gent (BE); KATHOLIEKE UNIVERSITEIT LEUVEN, K.U.LEUVEN R&D, Leuven (BE)

(72) Inventors: Diether Lambrechts, Kessel-Lo (BE); Line Heylen, Wilsele (BE); Ben Sprangers, Leuven (BE); Maarten Naesens, Leuven (BE)

(21) Appl. No.: 17/620,261

(22) PCT Filed: Jun. 17, 2020
(86) PCT No.: PCT/EP2020/066702

§ 371 Date: Dec. 17, 2021

(30) Foreign Application Priority Data
Jun. 17, 2019 (EP) 19180558.9

(51) Int. Cl. C12Q 1/6883

(52) U.S. Cl. CPC C12Q 1/6883; C12Q 2600/154

(57) ABSTRACT

The present invention relates to biomarkers for predicting the risk of developing chronic allograft injury in a patient, and means and methods for (post-transplant) preservation of allografts and transplantation organs. In particular, a method to predict the risk of developing chronic allograft injury in a patient is presented based on age-related increase of methylation of CpGs. In particular, the allograft is a kidney.

### CROSS-REFERENCE TO RELATED APPLICATIONS

This application is a national phase entry under 35 U.S.C. § 371 of International Patent Application PCT/EP2020/066702, filed Jun. 17, 2020, designating the United States of America and published in English as International Patent Publication WO 2020/254364 A1 on Dec. 24, 2020, which claims the benefit under Article 8 of the Patent Cooperation Treaty to European Patent Application Serial No. 19180558.9, filed Jun. 17, 2019, the entireties of which are hereby incorporated by reference.

### FIELD OF THE INVENTION

The present invention relates to biomarkers for predicting the risk of developing chronic allograft injury in a patient, and means and methods for (post-transplant) preservation of allografts and transplantation organs. In particular, a method to predict the risk of developing chronic allograft injury in a patient is presented based on age-related increase of methylation of CpGs. In particular, the allograft is a kidney.

### BACKGROUND

Kidney transplantation is the treatment of choice for patients with end-stage renal failure. Despite the development of potent immune suppressive therapies, which improve outcome early after transplantation, annually 3-5% of grafts show late graft failure, with devastating consequences for patient quality of life and survival. Chronic allograft injury (CAI) represents a leading cause for this late graft loss, and has been linked to ischemia-reperfusion injury (IRI) occurring during transplantation. In kidney transplantation, cold ischemia time is directly proportional to delayed functioning of grafted kidneys (Ojo et al. 1997, Transplantation 63:968-974), overall reduced allograft function (Salahudeen et al. 2004, Kidney Int 65:713-718), and CAI (Yilmaz et al. 2007, Transplantation 83:671-676). Experimental studies have highlighted that cold ischemia can trigger a complex set of events that delay graft function and sustain renal injury. For instance, acute ischemia can lead to chronic activation of the host immune response to the allograft (Perico et al. 2004, The Lancet 364:1814-1827). Immunological as well as non-immunological insults leading to interstitial fibrosis and tubular atrophy culminate in injury and kidney failure, which was shown to be correlated to DNA methylation changes (Bontha et al. 2017, Am J Transplant 17:3060-3075). Epigenome-wide studies assessing methylation levels to determine response to a specific cancer treatment has pinpointed a panel of specific methylation markers (Spinella et al. WO2014/025582A1). Chronic allograft injury or nephropathy predictive biomarkers based on differential gene expression levels identified so far all involve complex methods including mRNA analysis and therefore highly depend on timing of sampling and accuracy (for instance see Scherer, US2010/0022627A1 and Murphy et al. US2017/0114407A1). In fact, there are currently no biomarkers to predict CAI. So there is a need for reliable markers to determine or predict an increased risk of developing CAI, which in turn can assist in the development of treatments aimed at avoiding, inhibiting or restricting the development of CAI.

DNA methylation changes affecting the Ras oncoprotein inhibitor RASAL1 have been proposed to underlie kidney fibrosis, which is a key pathological feature contributing to chronic allograft injury (CAI) following kidney transplantation (Bechtel et al. 2010, Nat Med 16:544-550). Bontha et al. 2017 looked into DNA methylation in relation to kidney allograft IFTA (interstitial fibrosis and tubular atrophy) with the focus on the consequences of changes in DNA methylation on gene expression, the integration of both leading to identification of 3 miRNAs.

### SUMMARY OF THE INVENTION

The invention in one aspect relates to methods for predicting the risk of developing chronic kidney allograft injury, comprising the steps of:
obtaining DNA from a biological sample obtained from the allograft or from the recipient of the allograft;
detecting methylation on a set of CpGs in the DNA of the sample;
predicting the allograft to be at risk of developing chronic injury when the methylation detected on the set of CpGs is higher compared to reference values of methylation on the same set of CpGs;
   wherein the set of CpGs is comprising at least 4 CpGs chosen from the CpGs listed in Table 3, at least 4 CpGs chosen from the CpGs listed in Table 4, or at least 4 CpGs chosen from the CpGs listed in Tables 3 and 4. When said set of CpGs is comprising at least 4 CpGs chosen from the CpGs listed in Table 3, the risk of developing chronic injury can be defined as a risk of developing glomerulosclerosis. When said set of CpGs is comprising at least 4 CpGs chosen from the CpGs listed in Table 4, the risk of developing chronic injury can be defined as a risk of developing interstitial fibrosis.

The above methods can further comprise detecting, in the DNA of the sample, methylation on a CpG of a CpG island chosen from Table 5, on a CpG chosen from Table 6, or on a CpG chosen from Table 7. In particular, the above methods are further comprising detecting, in the DNA of the sample, methylation on a set of at least 4 CpGs chosen from Table 7.

Alternatively, the invention relates to methods for predicting the risk of developing chronic kidney allograft injury, comprising the steps of:
obtaining DNA from a biological sample obtained from the allograft or from the recipient of the allograft;
detecting methylation on a set of CpGs in the DNA of the sample;
predicting the allograft to be at risk of developing chronic injury when the methylation detected on the set of CpGs is higher compared to reference values of methylation on the same set of CpGs;
   wherein the set of CpGs is comprising at least 1 CpG chosen from the CpGs listed in Table 3, or at least 1 CpG chosen from the CpGs listed in Table 4; and is further comprising at least 1 CpG chosen from the CpGs of the CpG islands listed in Table 5, at least 1 CpG chosen from the CpGs listed in Table 6, or at least 1 CpG chosen from the CpGs listed in Table 7; and
   wherein the set of CpGs is comprising at least 4 CpGs chosen from the combination of the CpGs listed in Tables 3, 4, 6, and 7, and the CpGs of the CpG islands listed in Table 5.

In any of the above methods, the biological sample can be taken at the time of implantation, or can be taken post-implantation. In particular, said biological sample is a biopsy sample from an allograft, or is a liquid biopsy sample.

Any of the above methods may further comprise the step of selecting an inhibitor of hypermethylation or an inhibitor of fibrosis for use in preservation of the kidney allograft when the kidney allograft is predicted to be at risk of developing chronic injury. Such inhibitor of hypermethylation can be a stimulator of TET enzyme, such as an inhibitor of the BCAT1 enzyme. Such inhibitor of fibrosis may be azacytidine or a Jnk-inhibitor.

The invention further relates to the use of a set of CpGs in a method for predicting the risk of developing chronic kidney allograft injury according to any of the above methods, wherein the set of CpGs is comprising:
at least 4 CpGs chosen from the CpGs listed in Table 3, at least 4 CpGs chosen from the CpGs listed in Table 4, or at least 4 CpGs chosen from the CpGs listed in Tables 3 and 4;
at least 4 CpGs chosen from the CpGs listed in Table 3, at least 4 CpGs chosen from the CpGs listed in Table 4, or at least 4 CpGs chosen from the CpGs listed in Tables 3 and 4; and is further comprising a CpG of a CpG island chosen from Table 5, a CpG chosen from Table 6, or a CpG chosen from Table 7; or
at least 1 CpG chosen from the CpGs listed in Table 3, or at least 1 CpG chosen from the CpGs listed in Table 4; and is further comprising at least 1 CpG chosen from the CpGs of a CpG island listed in Table 5, at least 1 CpG chosen from the CpGs listed in Table 6, or at least 1 CpG chosen from the CpGs listed in Table 7;
wherein the set of CpGs is comprising at least 4 CpGs chosen from the combination of the CpGs listed in Tables 3, 4, 6, and 7, and the CpGs of the CpG islands listed in Table 5;
and wherein the set of CpGs is comprising at most 10000 CpGs.

The invention further encompasses kits, such as diagnostic kits, comprising oligonucleotides to detect DNA methylation on a set of CpGs, wherein the set of CpGs is comprising:
at least 4 CpGs chosen from the CpGs listed in Table 3, at least 4 CpGs chosen from the CpGs listed in Table 4, or at least 4 CpGs chosen from the CpGs listed in Tables 3 and 4;
at least 4 CpGs chosen from the CpGs listed in Table 3, at least 4 CpGs chosen from the CpGs listed in Table 4, or at least 4 CpGs chosen from the CpGs listed in Tables 3 and 4; and is further comprising a CpG of a CpG island chosen from Table 5, a CpG chosen from Table 6, or a CpG chosen from Table 7; or
at least 1 CpG chosen from the CpGs listed in Table 3, or at least 1 CpG chosen from the CpGs listed in Table 4; and is further comprising at least 1 CpG chosen from the CpGs of a CpG island listed in Table 5, at least 1 CpG chosen from the CpGs listed in Table 6, or at least 1 CpG chosen from the CpGs listed in Table 7;
wherein the set of CpGs is comprising at least 4 CpGs chosen from the combination of the CpGs listed in Tables 3, 4, 6, and 7, and the CpGs of the CpG islands listed in Table 5; and
wherein the set of CpGs is comprising at most 10000 CpGs.

In particular, such kits find their use for predicting the risk of developing chronic kidney allograft injury.

The invention further relates to stimulators of TET enzyme activity and/or to inhibitors of fibrosis for use in preservation of a kidney allograft, wherein a higher risk of developing chronic allograft injury was predicted according to the any of the above methods or kits according to the invention.

### DESCRIPTION OF THE FIGURES

The drawings described are only schematic and are non-limiting. In the drawings, the size of some of the elements may be exaggerated and not drawn on scale for illustrative purposes.

FIG. 1. Manhattan plot showing genome-wide logarithmic P-values of the association between DNA methylation at individual CpGs (n=803 663) across the renal genome and age, adjusted for gender, cold ischemia time and type of donation. The dotted line represents the P-value at the FDR value of 0.05.

FIG. 2. Volcano plot showing logarithmic P-values of changes in methylation at individual CpGs (n=803 663) with increase in age, as measured in 95 renal biopsies. Peaks gaining (to the right of the middle vertical dotted line) and losing (to the left of the middle vertical dotted line) methylation are highlighted at FDR <0.05 and P<0.05 (between horizontal dotted lines).

FIG. 3. Top canonical pathways and top upstream regulators among the genes with a differentially methylated region upon aging, left for the implantation cohort (based on 5445 DMRs), right for the post-reperfusion cohort (based on 10 274 DMRs). The significance levels are depicted on the y-axis. In the boxes, the number of genes with significant age-associated differentially methylated regions in the pathways are presented as percentage and ratio, respectively.

FIG. 4. Top canonical pathways and top upstream regulators among the genes whose promoters were either hyper- or hypomethylated upon aging in the implantation cohort. The significance levels are depicted on the y-axis. In the boxes, the number of genes with significant age-associated hyper- or hypomethylated promoters in the different pathways are presented as percentage and ratio, respectively.

FIGS. 5A-5B. Volcano plot showing logarithmic P-values of changes in methylation at age-associated CpGs with structural changes observed upon aging at baseline and at one year after transplantation. Peaks gaining (to the right of the middle vertical dotted line) and losing (to the left of the middle vertical dotted line) are highlighted at FDR <0.05 and P<0.05 (between horizontal dotted lines).

FIG. 6. Top canonical pathways and top upstream regulators among the age-associated differentially methylated genes whose promoter methylation correlates to future glomerulosclerosis and interstitial fibrosis, and to only future glomerulosclerosis. The significance levels are depicted on the y-axis. In the boxes, the number of significant genes in the different pathways are presented as percentage and ratio, respectively.

FIG. 7. Changes in methylation correlating with glomerulosclerosis at one year after transplantation, against the correlation with reduced renal allograft function (eGFR<45 ml/min/1.73 m2) at one year after transplantation. Colored points depict CpGs for which both correlations are significant at FDR<0.05, with blue used for the same direction of effect in both correlations and red for the inverse direction of effect.

### DETAILED DESCRIPTION TO THE INVENTION

The present invention will be described with respect to particular aspects and embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, N.Y. (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

Although it is known that DNA methylation levels change with age in various organs, the functional implications of increased DNA methylation on an organ are not known. In work leading to the present invention, genome-wide DNA methylation changes (in >800 000 CpG sites) were profiled in 95 renal biopsies obtained prior to kidney transplantation from donors aged 16 to 73 years. Donor age associated significantly with methylation of 92 778 CpGs (FDR<0.05), corresponding to 10 285 differentially methylated regions. Using an independent cohort of 67 biopsies, these findings were independently validated. Interestingly, methylation status of the 92 778 age-related CpG's was associated with glomerulosclerosis (34.4% of CpGs at FDR<0.05) and interstitial fibrosis (0.9%) and graft function at one year after transplantation, but not with tubular atrophy and arteriosclerosis. No association was observed with any of these pathologies at the time of transplantation (0% at FDR<0.05). Thus, age-associated organ DNA methylation status at the time of transplantation (a defined time-point) is predictive for future functioning and injury of transplanted organs.

Therefore, the invention in one aspect relates to several methods for predicting, determining, detecting, measuring, assessing or assaying the risk of developing chronic allograft injury. In particular to these methods, the allograft organ is a kidney. Such methods include those comprising e.g. the steps of:
obtaining or isolating DNA from a biological sample obtained from the allograft or from the recipient of the allograft;
detecting, determining, measuring, assessing or assaying methylation on a set of CpGs in the DNA of the sample;
predicting, determining, detecting, measuring, assessing or assaying the allograft to be at risk of developing chronic injury when the methylation detected on the set of CpGs is higher compared to reference values of methylation on the same set of CpGs;
   wherein the set of CpGs is comprising or at least 4 CpGs chosen from the CpGs listed in Table 3, or at least 4 CpGs chosen from the CpGs listed in Table 4, or at least 4 CpGs chosen from the CpGs listed in Tables 3 and 4. In particular, said set of CpGs is in one embodiment comprising at least 4 CpGs chosen from the CpGs listed in Table 3, and the risk of developing chronic injury can then be defined as a risk of developing (post-transplant) glomerulosclerosis and/or (post-transplant) interstitial fibrosis. In an alternative, said set of CpGs is in one embodiment comprising at least 4 CpGs chosen from the CpGs listed in Table 4, and the risk of developing chronic injury can then be defined as a risk of developing interstitial fibrosis.

The annotation “CpG” is an abbreviation for 5′-cytosine-phosphate-guanine-3′. Although the frequency of occurrence of CpGs in the human genome is less than 25% of the expected frequency, CpGs tend to cluster in “CpG islands”. One possible definition of a CpG island refers to a region of at least 200 bp in length with a GC-content of more than 50%, and with an observed-to-expected CpG ratio of more than 60%. Herein the observed CpG obviously is the actual number of CpG occurrences within the delineated CpG island. The expected number of CpGs can be calculated as ([C]×[G])/sequence length (Gardiner-Garden et al. 1987, J Mol Biol 196:261-282) or as (([C]+[G])/2)²/sequence length (Saxonov et al. 2006, PNAS 103:1412-1417), wherein [C] and [G] are the number of cytosines and guanines, respectively, in the delineated CpG island. As synonym for CpG island, reference is sometimes made to differentially methylated region or DMR.

“DNA methylation”, in particular methylation on a (set of) CpG(s) or methylation of a (set of) CpGs, is the attachment of a methyl group to the cytosine located in a (set of) CpG dinucleotide(s), creating a (set of) 5-methylcytosine(s) (5mC). CpG dinucleotides (CpGs) tend to cluster in so-called CpG islands, and when they are methylated this often correlates with transcriptional silencing of the affected gene. DNA methylation represents a relatively stable but reversible epigenetic mark (Bachman et al. 2014, Nat Chem 6:1049-1055). Its removal can be initiated by ten-eleven translocation (TET) enzymes, which convert 5mC to 5-hydroxymethylcytosine (5hmC) in an oxygen-dependent manner (Williams et al. 2011, Nature 473:343-348). Recently, it was demonstrated that tumor hypoxia reduces TET activity, leading to the accumulation of 5mC and loss of 5hmC (Thienpont et al. 2016, Nature 537:63-68). Assays for determining, detecting, measuring, assessing or assaying DNA methylation as well as methodologies for scoring DNA methylation levels (and changes therein) will be discussed in more detail further herein. The term “allograft” is used herein to define a transplant/transplantation of an organ or tissue from one individual to another of the same species (with a different genotype). For example, a transplant or translation of an organ or tissue from one person to another (not being an identical twin), is an allograft. Allografts account for many human transplants, including those from cadaveric donors, living related donors, and living unrelated donors. Allografts are also known as an allogeneic graft or a homograft. Allografts may consist of cells, tissue, or organs. An “allograft sample” or “sample of an allograft” may be obtained as a solid or liquid biopsy. A solid biopsy is normally comprising cells or tissue whereas a liquid biopsy is comprising any bodily fluid. More in particular, a liquid biopsy is comprising blood, serum or plasma, or is derived from blood, serum or plasma, in particular obtained from the recipient of the allograft. The advantage of a liquid biopsy is that it is non-invasive. Liquid biopsies taken from the blood usually comprise cell-free DNA (cfDNA) from different sources, including from transplanted donor organs, and therefore is increasingly studied as source of biomarkers (Knight et al. 2019, Transplantation 103:273-283). Methylation of cfDNA of tumor origin is being studies e.g. for purposes of detecting cancer (e.g. Nunes et al. 2018, Cancers 10:357). Although not yet routinely implemented, longitudinal surveillance biopsies post-transplant are being used as monitoring tool in some clinics for detection of often unsuspected graft injury such as to adjust post-transplant treatment and to individualize therapy in order to limit allograft injury (Henderson et al. 2011, Am J Transplant 11:1570-1575). In the clinical unit of Henderson et al. (ibidem), surveillance biopsies led to change in management in 56% of their patients. In case of the allograft being a kidney, basically two ways to perform a renal biopsy exist: percutaneous biopsy (renal needle biopsy) and open biopsy (surgical biopsy). The percutaneous biopsy is most common and employs a thin biopsy needle to remove kidney tissue wherein the needle may be guided using ultrasound or CT scan. For small renal tissue samples, a fine needle aspiration biopsy is possible, whereas for larger renal tissue samples, a needle core biopsy is obtained by e.g. using a spring-loaded needle. Liquid biopsies from a kidney can be taken by collecting e.g. blood or urine leaving the kidney, or by collecting urine; such liquid biopsies comprising DNA shedded from cells in the kidney.

Allograft injury is referred to herein as any type of injury to the transplanted origin (present prior to transplantation such as already present in the donor or occurring between retrieval of the organ from the donor and transplantation to the recipient, or inflicted as consequence of the transplantation surgery) and leading to long term damage affecting the functioning of the organ—referred to herein as chronic allograft damage or injury—and potentially ultimately leading to failure of the allograft. In the context of the present invention, particular types of chronic damage can be predicted including kidney/renal glomerulosclerosis and kidney/renal interstitial fibrosis. Glomerulosclerosis refers to scarring (fibrosis, deposit of extracellular matrix) of the glomeruli, the small blood vessels of the kidney that filter waste products from the blood. Another type of injury is hypoxia, and renal tubules may be highly susceptible in view of their high oxygen consumption (Hewitson et al. 2012, Fibrogenesis & Tissue Repair 5(Suppll): S14). Hypoxia or ischemia may occur as consequence of ongoing kidney disease, but also as consequence of the transplantation procedure. It is usually the result of obstruction or cessation of blood flow to a tissue, for instance as a result from vasoconstriction, thrombosis or embolism, or because of removal from a (living or deceased) donor, resulting in limited supply of oxygen and nutrients, and if prolonged, in impairment of energy metabolism and cell death. Restoration of the blood flow, called “reperfusion”, results in oxygen reintroduction and a burst of ROS, leading to cell death associated with inflammation (Jouan-Lanhouet et al., 2014; Vanlangenakker et al., 2008; Halestrap, 2006). Ischemia can occur acutely, as during surgery, or from trauma to tissue incurred in accidents or by injuries, or following harvest of organs intended for subsequent transplantation, for example. When ischemia is ended by the restoration of blood flow, a second series of injuries events ensue, producing additional injury. Thus, whenever there is a transient decrease or interruption of blood flow in a subject, the resultant injury involves two-components, the direct injury occurring during the ischemic interval, and the indirect or reperfusion injury that follows, therefore named “ischemia-reperfusion injury (IRI)”. Chronic allograft injury (CAI) is common after kidney transplantation in which immunological (e.g., acute and chronic cellular and antibody-mediated rejection) and non-immunological factors (e.g., donor-related factors, ischemia—reperfusion injury, polyoma virus, hypertension, and calcineurin inhibitor nephrotoxicity) have a role. Despite the new Banff pathological classification, histopathological diagnosis is still far from being the ‘gold standard’ to understand the exact mechanisms in the development of CAI, which may lead to appropriate treatment (Akalin & O'Connell 2010, Kidney Int 78 (Suppl 119), S33-S37).

Predicting, determining, detecting, measuring, assessing or assaying an allograft to be at risk of developing chronic injury in general refers to any procedure relying on the status of markers or biomarkers that have predictive power for predicting, determining, measuring, assessing or assaying whether or not chronic injury will occur to the allograft in the future. In particular the status of such markers or biomarkers does not, or does not necessarily, provide information of the condition of the allograft at the moment of running the said procedure but does provide information on how the condition of the allograft is likely to develop over time, such as three months to one year after running the said procedure. Thus, by running such procedure, information is becoming available that is highly useful in the follow-up of subjects having received an allograft (allograft recipients) and assisting in the post-transplant management of these subjects/recipients. Such procedures can also be employed in the setting of clinical trials evaluating the effect of therapeutic compounds aiming at preserving the allograft or aiming at treating, inhibiting or preventing chronic allograft injury, or aiming at preservation of the allograft. The term “treatment” or “treating” or “treat” can be used interchangeably and is defined by a therapeutic intervention that slows, interrupts, arrests, controls, stops, reduces, or reverts the progression or severity of a sign, symptom, disorder, condition, injury, or disease, but does not necessarily involve a total elimination of all disease-related signs, symptoms, conditions, or disorders. The term “preservation” in the present context relates to allograft or organ preservation, and refers to any procedure or intervention supporting, maintaining, keeping, or ensuring, at any stage, the proper functioning of the allograft or organ.

Previously, correlations were established between the methylation status of CpGs as consequence of allograft ischemia (prior to transplantation) and future, long-term functioning of a kidney/renal allograft (Heylen et al. 2018, J Am Soc Nephrol 29:1566-1576; PCT/EP2018/086509, published as WO2019/122303; see Example 2 herein, which is taken from the Examples of PCT/EP2018/086509, published as WO2019/122303). In particular, a correlation was established with future kidney/renal interstitial fibrosis and glomerulosclerosis. These CpGs are listed herein as the CpGs occurring in the CpG islands listed in Table 5, or as the CpGs as listed in Tables 6 and 7. Table 5 refers to 66 CpG islands together covering 1634 CpGs, Table 6 refers to 413 CpGs selected from the said 1634 CpGs (26.4%), and Table 7 refers to 29 CpGs being a further selection from the said 413 CpGs (1.77% of the 1634 CpGs; 7% of the 413 CpGs). Example 2.5 concludes that determining the ischemia-induced methylation status of 4 CpGs from Table 7 (current numbering) is sufficient to predict future/chronic allograft injury.

In the context of the present invention, an unprecedented correlation was established between the methylation state of a particular and limited set of age-associated CpGs in the DNA of an allograft and the future, long-term (long time between assessment of the methylation status of these age-associated CpGs and the clinical outcome) functioning of a kidney/renal allograft. An “age-associated CpG” refers to the methylation status of a CpG or to the level of methylation on/of a CpG that correlates with age. In particular, the level of methylation on/of the age-associated CpGs in the DNA of an allograft referred to herein is increasing (also referred to as hypermethylated) with increasing age, or is decreasing (also referred to as hypomethylated) with increasing age. In particular, the methylation status of one set of (age-associated) CpGs in the DNA of an allograft was found to correlate with future glomerulosclerosis in the allograft (CpGs listed in Table 3; which are the top 50, or 0.16% of the 31805 (34.4% of all identified age-associated CpGs) differentially methylated CpG sites correlated with glomerulosclerosis), and the methylation status of another set of (age-associated) CpGs in the DNA of an allograft was found to correlate with future interstitial fibrosis in the allograft (CpGs listed in Table 4; which are the top 50, or 5.7% of the 880 (0.9% of all identified age-associated CpGs) differentially methylated CpG sites correlated with glomerulosclerosis). The CpGs as listed in Tables 3 and 4 all were resulting from further analysis of a larger set of CpGs for which their methylation status was correlated with age; in particular, a high level of methylation in these CpGs in the allograft is predictive for an increased risk of developing chronic allograft injury. In view of the conclusion of Example 2.5, it appears plausible that determining the methylation status of 4 CpGs from Table 3 and/or Table 4 is likewise sufficient to predict future/chronic allograft injury. In addition, the age-related CpG markers in the DNA of an allograft as identified herein as correlating with future/chronic allograft injury (Tables 3 and 4) can be combined with the previously identified ischemia-induced CpG markers (Tables 5-7) identified to correlate with future/chronic allograft injury. Thus, determining, detecting, measuring, assessing or assaying the methylation status of any such combination of 4 CpGs from any of Tables 3 to 7 is likewise sufficient to predict future/chronic allograft injury; and any such combinations comprising at least 1 CpG marker as defined or listed in Table 3 or 4 is part of the current invention. All of the CpGs (as listed in Tables 1, 3, 4, 6, 7) or CpG island (as listed in Tables 2, 5) were defined by their respective positions on the indicated chromosomes as annotated in the Genome Reference Consortium Human Hg19 Build #37 assembly. Retrieving the actual nucleic acid sequence from the indicated allocation on the indicated chromosome is known to the skilled person, and the actual nucleic acid sequence can be retrieved e.g. by using a genome browser (e.g. https://genome.ucsc.edu/ or https://www.ncbi.nlm.nih.gov/genome/). For Example, when using the Genome Browser available via https://genome.ucsc.edu/, by selecting as Human Assembly “Feb.2009(GRCh37/hg19)” (i.e. the Human Assembly as relied on in the Examples, see Example 1.1.4 and Example 2.4), and by querying the Position/Search Term “chr13:92050718-92050725” (i.e. region of chromosome 13 that should comprise the first listed CpG, cg03036557, of Table 1), the sequence “AT [symbol] ATGT” is retrieved—positions 92050720 (see column “pos” in Table 1)-92050721 herein correspond to the CpG sequence (bold, italic, underlined in the retrieved sequence) of cg03036557.

Therefore, the invention, in relating to several methods for predicting, determining, detecting, measuring, assessing or assaying the risk of developing chronic allograft injury includes methods comprising e.g. the steps of:

obtaining or isolating DNA from a biological sample obtained from the allograft or from the recipient of the allograft;
determining, detecting, measuring, assessing or assaying methylation on a set of CpGs in the DNA of the sample;
predicting, determining, detecting, measuring, assessing or assaying the allograft to be at risk of developing chronic injury when the methylation detected on the set of CpGs is higher compared to reference values of methylation on the same set of CpGs;
   wherein the set of CpGs is comprising or at least 4 CpGs chosen from the CpGs listed in Table 3, or at least 4 CpGs chosen from the CpGs listed in Table 4, or at least 4 CpGs chosen from the CpGs listed in Tables 3 and 4; and
   wherein these methods further comprise determining, detecting, measuring, assessing or assaying, in the DNA of the sample:
methylation on a CpG of a CpG island chosen from Table 5, on a CpG chosen from Table 6, or on a CpG chosen from Table 7: or
methylation on a set of at least 4 CpGs chosen from Table 7

In particular, said set of CpGs is in one embodiment comprising at least 4 CpGs chosen from the CpGs listed in Table 3, and the risk of developing chronic injury can then be defined as a risk of developing glomerulosclerosis. In an alternative, said set of CpGs is in one embodiment comprising at least 4 CpGs chosen from the CpGs listed in Table 4, and the risk of developing chronic injury can then be defined as a risk of developing interstitial fibrosis. In these embodiments, the defined risk may in particular be predicted or determined based on the results obtained with the set of CpGs selected from Table 3 or Table 4, respectively, only (thus not taking into account the results obtained with the additional CpG(s) selected from Tables 5, 6, and/or 7).

The invention, in relating to several methods for predicting, determining, detecting, measuring, assessing or assaying the risk of developing chronic allograft injury further includes methods comprising e.g. the steps of:
obtaining or isolating DNA from a biological sample obtained from the allograft or from the recipient of the allograft;
determining, detecting, measuring, assessing or assaying methylation on a set of CpGs in the DNA of the sample;
predicting, determining, detecting, measuring, assessing or assaying the allograft to be at risk of developing chronic injury when the methylation detected on the set of CpGs is higher compared to reference values of methylation on the same set of CpGs;
   wherein the set of CpGs is comprising or at least 1 CpG chosen from the CpGs listed in Table 3, or at least 1 CpG chosen from the CpGs listed in Table 4; and is further comprising at least 1 CpG chosen from the CpGs of the CpG islands listed in Table 5, at least 1 CpG chosen from the CpGs listed in Table 6, or at least 1 CpG chosen from the CpGs listed in Table 7; and
   wherein the set of CpGs is comprising at least 4 CpGs chosen from the combination of the CpGs listed in Tables 3, 4, 6, and 7, and the CpGs of the CpG islands listed in Table 5.

In any of the several methods for predicting, determining, detecting, measuring, assessing or assaying the risk of developing chronic allograft injury as described hereinabove, the allograft in particular is a kidney allograft. Furthermore, the sample of the allograft may be taken at the time of implantation in the recipient subject, or is taken post-implantation from the subject (e.g. 1 week, 2 weeks, 3 weeks or 4 weeks post-implantation, or up to 1, 2, or 3 months post-transplantation, or 3 months post-transplantation). In particular, such allograft sample is a biopsy sample from the allograft, or is a liquid biopsy sample.

The prediction, determination, detection, assessment or attribution of a ‘higher risk’ for chronic allograft injury or ‘higher risk’ of developing chronic allograft injury may be a 2-fold higher risk, a 3-fold, 4-fold or 5-fold higher risk, or a 10%, 20%, 30%, 40%, 50%, 60%, 70%, 80%, 90%, or 100% or more higher risk, as compared to the population of allografts displaying reference or control DNA or CpG methylation levels (see further). In general the risk of developing chronic allograft injury is increasing with the increase in DNA or CpG methylation levels on/of the set of CpGs as defined herein compared to the control or reference DNA or CpG methylation levels on/of the same set of CpGs; i.e. the higher the difference in DNA or CpG methylation, the higher the risk for chronic allograft injury or for developing chronic allograft injury.

Hypermethylation can be reversed by means of therapeutic intervention. Several compounds are used as methylation inhibitors, mainly in the field of cancer and in hypoxic tumors. Non-limiting examples comprise 5-azacytidine (AZA), a cytidine analog which is used for demethylation and also approved (as Vidaza) for treatment of myelodysplastic syndrome or other cancers, and decitabine (DEC) (Licht et al. 2015, Cell 162:938). Furthermore, by modulating the TET enzyme activity, compounds such as α-ketoglutarate, a cofactor of the TET enzymes, may also act in inhibiting DNA methylation under hypoxic or anoxic conditions. Thus, a stimulator of TET enzyme activity can be used for preservation or treatment of the allograft prior or post transplantation, when a higher risk of developing chronic allograft injury in a patient was predicted for said allograft, according to any of the hereinabove described methods for predicting or determining the risk of developing chronic allograft injury. The TET enzyme is converting methylated cytosine (5mC) into hydroxymethylated cytosine (5hmC), a reaction which is inhibited upon oxygen shortage. So stimulation of the TET enzyme activity may also be accomplished by oxygenation. In one embodiment, a method for preservation of the allograft comprises reverting hypermethylation of CpGs in the allograft by oxygenation. In another embodiment, stimulation of TET activity is established via acting on or modulating another enzyme that affects TET activity. For instance, in one embodiment, said stimulator of TET activity for use in preservation of allograft prior to transplantation is a modulator or inhibitor of BCAT1 activity. In fact, BCAT activity results reversible transamination of an α-amino group from branched-chain amino acids (BCAAs; i.e. valine, leucine and isoleucine) to α-ketoglutarate (αKG), which is a critical regulator of its own intracellular homeostasis and essential as cofactor for αKG-dependent dioxygenases such as the TET enzyme family (Raffel et al. 2017, Nature 551:384). By reducing the activity of BCAT1, intracellular αKG levels increase, thereby stimulating TET, resulting in inhibition of 5mC formation or DNA methylation. Recently, the role of BCAT1 in macrophages has been investigated, and the BCAT1-specific inhibitor, ERG240, a leucine analogue, showed reduced inflammation through a decrease of macrophage infiltration in for instance kidneys (Papathanassia et al. 2017, Nat Commun 8:16040). These findings all together allow to conclude that such BCAT1 inhibitors represent an alternative in the treatment needed to preserve allografts, via a mechanism acting on inhibition of hypermethylation.

Preclinical work has identified e.g. azacytidine and Jnk-inhibitors as having the potential to halt kidney fibrosis (Bechtel 2010, Nat Med 16:544; Yang 2010, Nat Med 16:535). Demethylating agents are likewise considered in the treatment of chronic or diabetic kidney disease (Larkin et al. 2018, FASEB 1 32:5215).

Any of the several methods for predicting, determining, detecting, measuring, assessing or assaying the risk of developing chronic allograft injury as described hereinabove may further be comprising a step of selecting an inhibitor of hypermethylation or an inhibitor of fibrosis for use in preservation of the kidney allograft when the kidney allograft is predicted to be at risk of developing chronic injury. Examples of inhibitors of hypermethylation include stimulators of the TET enzyme, such as inhibitors of the BCAT1 enzyme. Examples of inhibitors of fibrosis are azacytidine (or other demethylating agents) and ink-inhibitors.

In another aspect of the invention, stimulators of TET enzyme activity or inhibitors of fibrosis (in particular of kidney or renal fibrosis), demethylating agents, or inhibitors of hypermethylation for use in preservation of a kidney allograft are envisaged, in particular in conjunction with the prediction or determination of a higher risk of developing chronic allograft injury according to any of the several methods for predicting, determining, detecting, measuring, assessing or assaying the risk of developing chronic allograft injury as described hereinabove. Thus, the invention relates to: (a) stimulators of TET enzyme activity or inhibitors of fibrosis and/or demethylating agents for use in preservation of a kidney allograft, (b) use of a stimulator of TET enzyme activity, of an inhibitor of fibrosis and/or of a demethylating agent for use in the manufacture of a medicament for preserving of a kidney allograft, or (c) methods for preserving a kidney allograft, comprising:
obtaining or isolating DNA from a biological sample obtained from the allograft or from the recipient of the allograft;
determining, detecting, measuring, assessing or assaying methylation on a set of CpGs in the DNA of the sample;
predicting, determining, detecting, measuring, assessing or assaying the allograft to be at risk of developing chronic injury when the methylation detected on the set of CpGs is higher compared to reference values of methylation on the same set of CpGs;
administering a stimulator of TET enzyme activity, an inhibitors of fibrosis, and/or a demethylating agent to the recipient of the allograft;
   wherein the set of CpGs is comprising:
or at least 4 CpGs chosen from the CpGs listed in Table 3, or at least 4 CpGs chosen from the CpGs listed in Table 4, or at least 4 CpGs chosen from the CpGs listed in Tables 3 and 4;
or at least 4 CpGs chosen from the CpGs listed in Table 3, or at least 4 CpGs chosen from the CpGs listed in Table 4, or at least 4 CpGs chosen from the CpGs listed in Tables 3 and 4; and is further comprising a CpG of a CpG island chosen from Table 5, a CpG chosen from Table 6, or a CpG chosen from Table 7; or
or at least 1 CpG chosen from the CpGs listed in Table 3, or at least 1 CpG chosen from the CpGs listed in Table 4; and is further comprising at least 1 CpG chosen from the CpGs of a CpG island listed in Table 5, at least 1 CpG chosen from the CpGs listed in Table 6, or at least 1 CpG chosen from the CpGs listed in Table 7; wherein the set of CpGs is comprising at least 4 CpGs chosen from the combination of the CpGs listed in Tables 3, 4, 6, and 7, and the CpGs of the CpG islands listed in Table 5.

The invention further relates to uses of sets of CpGs in any of the several methods for predicting, determining, detecting, measuring, assessing or assaying the risk of developing chronic allograft injury as described hereinabove, wherein such sets of CpGs e.g. are comprising:
or at least 4 CpGs chosen from the CpGs listed in Table 3, or at least 4 CpGs chosen from the CpGs listed in Table 4, or at least 4 CpGs chosen from the CpGs listed in Tables 3 and 4;
or at least 4 CpGs chosen from the CpGs listed in Table 3, or at least 4 CpGs chosen from the CpGs listed in Table 4, or at least 4 CpGs chosen from the CpGs listed in Tables 3 and 4; and is further comprising a CpG of a CpG island chosen from Table 5, a CpG chosen from Table 6, or a CpG chosen from Table 7; or
or at least 1 CpG chosen from the CpGs listed in Table 3, or at least 1 CpG chosen from the CpGs listed in Table 4; and is further comprising at least 1 CpG chosen from the CpGs of a CpG island listed in Table 5, at least 1 CpG chosen from the CpGs listed in Table 6, or at least 1 CpG chosen from the CpGs listed in Table 7; wherein the set of CpGs is comprising at least 4 CpGs chosen from the combination of the CpGs listed in Tables 3, 4, 6, and 7, and the CpGs of the CpG islands listed in Table 5.

The invention further relates to kits, such a diagnostic kits or theranostic kits, comprising tools to detect, determine, measure, assess or assay methylation on/of (sets of) CpGs subject of the invention. In particular such tools are oligonucleotides capable of detecting, determining, measuring, assessing or assaying DNA methylation on/of (sets of) CpGs of the invention; other reagents are, however, not excluded from being part of the kit. Oligonucleotides for instance are primers and/or probes (one or more of them optionally provided on any type of solid support; and one or more of the primers or probes provided may comprise any type of detectable label) targeting the CpGs of the intended set of CpGs. A further reagent part of the kit may be one or more of a bisulfite reagent, an artificially generated methylation standard, a methylation-dependent restriction enzyme, a methylation-sensitive restriction enzyme, and/or PCR reagents. The kit may also comprise an insert or leaflet with instructions on how to operate the kit. The kit may further comprise a computer-readable medium that causes a computer to compare methylation levels from an allograft sample at the selected CpG loci to one or more control or reference profiles and computes a prediction value form the difference in CpG methylation in the allograft sample and the control profile. In an embodiment, the computer readable medium obtains the control or reference profile from historical methylation data for an allograft or patient or pool of allografts or patients. In some embodiments, the computer readable medium causes a computer to update the control or reference based on the testing results from the testing of a new allograft sample. In particular, such kits are used in in any of the several methods for predicting, determining, detecting, measuring, assessing or assaying the risk of developing chronic allograft injury as described hereinabove. In one particular embodiment, oligonucleotides capable of detecting, determining, measuring, assessing or assaying DNA methylation are used in allele-specific amplification or primer extension methods. These reactions typically involve use of primers that are designed to specifically target a polymorphism (such as the cytosine or thymidine of a CpG after bisulfite conversion) via a mismatch at the 3′-end of a primer. The presence of a mismatch effects the ability of a polymerase to extend a primer when the polymerase lacks error-correcting activity. If the 3′-terminus is mismatched, the extension is impeded. In some embodiments, the oligonucleotide is used in conjunction with a second primer in an amplification reaction. The second primer hybridizes at a site up- or downstream/in the vicinity of the CpG of interest. Amplification proceeds from the two primers leading to a detectable product signifying the particular allelic form is present. In a further particular embodiment, oligonucleotides capable of detecting, determining, measuring, assessing or assaying DNA methylation are used as allele-specific probes (e.g. designed to discriminate between cytosine or thymidine of a CpG after bisulfite conversion); such probes usually incorporate a label detectable in some way (many variations are known and available to the skilled person).

More in particular, such kits are kits comprising oligonucleotides to detect, determine, measure, assess or assay DNA methylation on a set of CpGs, wherein the set of CpGs is e.g. comprising:
or at least 4 CpGs chosen from the CpGs listed in Table 3, or at least 4 CpGs chosen from the CpGs listed in Table 4, or at least 4 CpGs chosen from the CpGs listed in Tables 3 and 4;
or at least 4 CpGs chosen from the CpGs listed in Table 3, or at least 4 CpGs chosen from the CpGs listed in Table 4, or at least 4 CpGs chosen from the CpGs listed in Tables 3 and 4; and is further comprising a CpG of a CpG island chosen from Table 5, a CpG chosen from Table 6, or a CpG chosen from Table 7; or
or at least 1 CpG chosen from the CpGs listed in Table 3, or at least 1 CpG chosen from the CpGs listed in Table 4; and is further comprising at least 1 CpG chosen from the CpGs of a CpG island listed in Table 5, at least 1 CpG chosen from the CpGs listed in Table 6, or at least 1 CpG chosen from the CpGs listed in Table 7;
   wherein the set of CpGs is comprising at least 4 CpGs chosen from the combination of the CpGs listed in Tables 3, 4, 6, and 7, and the CpGs of the CpG islands listed in Table 5.

As indicated above, such kits find their particular use in predicting, determining, detecting, measuring, assessing or assaying the risk of developing chronic kidney allograft injury.

In a particular embodiment to all of the methods, uses and kits of the invention as outlined hereinabove, the sets of CpGs referred to therein are comprising at least 4 CpGs, at least 5 CpGs, at least 6 CpGs, at least 7 CpGs, at least 8 CpGs, at least 9 CpGs, at least 10 CpGs, at least 11 CpGs, at least 12 CpGs, at least 13 CpGs, at least 14 CpGs, at least 15 CpGs, at least 16 CpGs, at least 17 CpGs, at least 18 CpGs, at least 19 CpGs, at least 20 CpGs; or are comprising between 4 and 10000 CpGs, between 4 and 7500 CpGs, between 4 and 5000 CpGs, between 4 and 4000 CpGs, between 4 and 3000 CpGs, between 4 and 2000 CpGs, between 4 and 1000 CpGs, between 4 and 900 CpGs, between 4 and 800 CpGs, between 4 and 700 CpGs, between 4 and 600 CpGs, between 4 and 500 CpGs, between 4 and 400 CpGs, between 4 and 300 CpGs, between 4 and 200 CpGs, between 4 and 100 CpGs, between 4 and 90 CpGs, between 4 and 80 CpGs, between 4 and 70 CpGs, between 4 and 60 CpGs, between 4 and 50 CpGs, between 4 and 40 CpGs, between 4 and 30 CpGs, between 4 and 20 CpGs, or between 4 and 10 CpGs; or a most 10000 CpGs, at most 7500 CpGs, at most 5000 CpGs, at most 4000 CpGs, at most 3000 CpGs, at most 2000 CpGs, at most 1000 CpGs, at most 900 CpGs, at most 800 CpGs, at most 700 CpGs, at most 600 CpGs, at most 500 CpGs, at most 400 CpGs, at most 300 CpGs, at most 200 CpGs, at most 100 CpGs, at most 90 CpGs, at most 80 CpGs, at most 70 CpGs, at most 60 CpGs, at most 50 CpGs, at most 40 CpGs, at most 30 CpGs, at most 20 CpGs, or at most 10 CpGs. In a further embodiment, where the set of CpGs is comprising at least 1 CpG chosen from the CpGs listed in Table 7, the selected CpG is cg01811187, is cg17078427, is cg16547027, is cg19596468, is cg14309111, is cg17603502, is cg08133931, is cg18599069, is cg24840099, is cg09529433, is cg10096645, is cg06108383, is cg03884082, is cg01065003, is cg22647713, is cg20449692, is cg07136023, is cg20811659, is cg20048434, is cg06546607, is cg00403498, is cg20891301, is cg17416730, is cg01724566, is cg16501308, is cg06230736, is cg03199651, is cg06329022, or is cg13879776.

In a particular embodiment to all of the methods, uses and kits of the invention as outlined hereinabove, the detection, determination, measurement, assaying or assessment of the methylation on/of a set of CpGs in the DNA of a biological sample obtained from the allograft or from the recipient of the allograft, the total number of CpGs in the set of CpGs is at least 4 CpGs, at least 5 CpGs, at least 6 CpGs, at least 7 CpGs, at least 8 CpGs, at least 9 CpGs, at least 10 CpGs, at least 11 CpGs, at least 12 CpGs, at least 13 CpGs, at least 14 CpGs, at least 15 CpGs, at least 16 CpGs, at least 17 CpGs, at least 18 CpGs, at least 19 CpGs, at least 20 CpGs; or are comprising between 4 and 10000 CpGs, between 4 and 7500 CpGs, between 4 and 5000 CpGs, between 4 and 4000 CpGs, between 4 and 3000 CpGs, between 4 and 2000 CpGs, between 4 and 1000 CpGs, between 4 and 900 CpGs, between 4 and 800 CpGs, between 4 and 700 CpGs, between 4 and 600 CpGs, between 4 and 500 CpGs, between 4 and 400 CpGs, between 4 and 300 CpGs, between 4 and 200 CpGs, between 4 and 100 CpGs, between 4 and 90 CpGs, between 4 and 80 CpGs, between 4 and 70 CpGs, between 4 and 60 CpGs, between 4 and 50 CpGs, between 4 and 40 CpGs, between 4 and 30 CpGs, between 4 and 20 CpGs, or between 4 and 10 CpGs; or a most 10000 CpGs, at most 7500 CpGs, at most 5000 CpGs, at most 4000 CpGs, at most 3000 CpGs, at most 2000 CpGs, at most 1000 CpGs, at most 900 CpGs, at most 800 CpGs, at most 700 CpGs, at most 600 CpGs, at most 500 CpGs, at most 400 CpGs, at most 300 CpGs, at most 200 CpGs, at most 100 CpGs, at most 90 CpGs, at most 80 CpGs, at most 70 CpGs, at most 60 CpGs, at most 50 CpGs, at most 40 CpGs, at most 30 CpGs, at most 20 CpGs, or at most 10 CpGs.

In a particular embodiment to all of the methods, uses and kits of the invention as outlined hereinabove, the detection, determination, measurement, assaying or assessment of the methylation on/of a set of CpGs in the DNA of a biological sample obtained of the allograft or of the recipient of the allograft, is involving extraction of the DNA from the biological sample. Such DNA can be cell-free DNA (cfDNA) as described hereinabove.

In a particular embodiment to all of the methods, uses and kits of the invention as outlined hereinabove, the detection, determination, measurement, assaying or assessment of the methylation on/of a set of CpGs in the DNA of a biological sample obtained of the allograft or of the recipient of the allograft, is involving treatment of the DNA with bisulfite and further, optionally, amplifying the bisulfite-treated genomic DNA with primers specific for each of CpGs in the set of CpGs.

In a particular embodiment to all of the methods, uses and kits of the invention as outlined hereinabove, the methylation on/of a set of CpGs in the DNA of a biological sample obtained of the allograft or of the recipient of the allograft, can be detected, determined, measured, assayed or assessed by methylation-specific PCR, quantitative methylation-specific PCR, methylation-sensitive DNA restriction enzyme analysis, quantitative bisulfite pyrosequencing, bisulfite genomic sequencing PCR, TAB-seq, TAPS, RRBS or cf-RRBS.

In a particular embodiment to all of the methods, uses and kits of the invention as outlined hereinabove, the detection, determination, measurement, assaying or assessment of the methylation on/of a set of CpGs in the DNA of a biological sample obtained of the allograft or of the recipient of the allograft, is involving extraction of the DNA or cfDNA from the biological sample, and/or treatment of the DNA with bisulfite, and/or methylation-specific PCR, quantitative methylation-specific PCR, methylation-sensitive DNA restriction enzyme analysis, quantitative bisulfite pyrosequencing, bisulfite genomic sequencing PCR, TAB-seq, TAPS, RRBS or cf-RRBS.

DNA Methylation Level

Although sequences in the human genome other than CpG are prone to DNA methylation such as CpA and CpT (see Ramsahoye 2000, Proc Natl Acad Sci USA 97:5237-5242; Salmon and Kaye 1970, Biochim Biophys Acta 204:340-351; Grafstrom 1985, Nucleic Acids Res 13:2827-2842; Nyce 1986, Nucleic Acids Res 14:4353-4367; Woodcock 1987, Biochem Biophys Res Commun 145:888-894), the methylation state is typically determined in CpG sequences. The methylation detected, determined, measured, assayed or assessed on/of CpGs of the DNA of an allograft sample according to any of the methods described hereinabove is referred to also as DNA methylation level. The terms “determining”, “detecting”, “measuring,” “assessing,” and “assaying” are used interchangeably and include both quantitative and qualitative determinations.

Differences in DNA methylation levels/CpG methylation levels can be compared between samples. An increase in the DNA methylation level can for instance refer to a value that is at least 10% higher, at least 20% higher, or at least 30% higher, at least 40% higher, at least 50% higher, at least 60% higher, at least 70% higher, at least 80% higher, at least 90% higher, or more than 100% higher, or at least 2-fold, or at least 3-fold, or more than 4-fold higher than the methylation level of the reference value of methylation (as long as methylation on/of the same DNA methylation sites/same CpGs are compared), or more specifically than the methylation level of the lower tertile of the reference allograft organ population.

The DNA methylation level can alternatively be used to calculate a methylation risk score (MRS), which is compared to one or more control MRS values. A “methylation risk score”, “DNA methylation score”, “risk score”, or “methylation score”, as used interchangeably herein, may be developed and/or calculated via several formulas, and is based in the methylation level or value of a number of CpGs. One example of a method for MRS calculation is provided by Ahmad et al. 2016 (Oncotarget 7:71833) being developed from the multivariate Cox model. Another MRS calculation method as used herein is explained in Example 2.6.4 herein). A person skilled in the art will be aware of applicable formulas and models for implementation and development of the MRS of the present method of the invention. Once the MRS is obtained for an allograft sample, the prediction of the outcome or higher risk of developing chronic allograft injury is dependent on a comparison of said MRS to a reference population, or the MRS of a reference population, or the average or mean MRS of a reference population. Said reference population comprises allograft samples from a population of subjects with a mixtures of high and low MRS values, representing healthy high-quality and damaged low-quality allografts or donor organs, which can be ranked and classified according to the MRS value. Such MRS values can be divided in e.g. terciles or tertiles (3), quartiles (4), quintiles (5), sextiles (6), septiles (7), octiles (8) or deciles (10), and reference MRS values can e.g. consist of the lower tertile, quartile, . . . , decile, etc.

The control or reference DNA or CpG methylation level may be a reference value and/or may be derived from one or more samples, an average or mean MRS may be used, optionally from historical methylation data for a patient/allograft or pool of patients or pool of allografts. In function of the number of sample values available, the control or reference DNA or CpG methylation levels may be adjusted. It will be understood that the control may also represent an average of the methylation levels or an average of the MRS for a group of samples or patients, in particular for a group of samples from organs which are the same as the allografted organ.

As a further alternative allowing comparison of DNA or CpG methylation levels, the methylation β values (as an estimate of methylation level using the ratio of intensities between methylated and unmethylated alleles. β values range between 0 and 1, with β=0 being unmethylated and β=1 being fully methylated), can be used. In particular, DNA methylation β values of a CpG is determined, and β values higher than those determined for control or reference DNA or CpG methylation are indicative of an increased risk of developing chronic allograft injury.

DNA methylation β values for each CpG of a set of CpGs can be determined, and an increased risk of developing chronic allograft injury can either be determined as requiring a higher β values for each of the individual CpG compared to the reference or control β value for each individual CpG, or it can be determined as requiring a higher average β value calculated starting from the β values of the individual CpGs compared to the average reference or control β value calculated starting from the reference or control β values of the individual CpGs. In particular, an increased risk of developing chronic allograft injury can be predicted when those β values (whether per individual CpG or as average of a set of CpGs) are at least 0.025 higher in the allograft as compared to the control or reference β values. Alternatively, said β values are at least 0.05, at least 0.075, at least 0.1, at least 0.125, at least 0.15, at least 0.175, at least 0.2, at least 0.2125, at least 0.225, at least 0.25, at least 0.275, at least 0.3, at least 0.325, at least 0.35, or at least 0.375 higher in the set of CpGs as compared to the control or reference β values.

DNA Methylation Assays

Assays for DNA methylation analysis have been reviewed by e.g. Laird 2010 (Nat Rev Genet 11:191-203). The main principles of possible sample pretreatment involve enzyme digestion (relying on restriction enzymes sensitive or insensitive to methylated nucleotides), affinity enrichment (involving e.g. chromatin immunoprecipitation, antibodies specific for 5MeC, methyl-binding proteins), sodium bisulfite treatment (converting an epigenetic difference into a genetic difference) followed by analytical steps (locus-specific analysis, gel-based analysis, array-based analysis, next-generation sequencing-based analysis) optionally combined in a comprehensible matrix of assays. Laird 2010 is providing a plethora of bioinformatic resources useful in DNA methylation analysis which can be applied by the skilled person as guiding principles, when wishing to analyze the methylation status of up to about 100 CpGs in a sample, with assays such as MethyLight, EpiTYPER, MSP, COBRA, Pyrosequencing, Southern blot and Sanger BS appearing to be the most suitable assays. This guidance does, however, not take into account that assays with higher coverage can be adapted towards lower coverage. For example, design of custom DNA methylation profiling assays covering up to 96 or up to 384 individual regions is possible e.g. by using the VeraCode® technology provided by IIlumina® (compared to the 450K DNA methylation array covering approximately 480000 individual CpGs). Another such adaptation for instance is enrichment of genome fractions comprising methylation regions of interest which is possible by e.g. hybridization with bait sequences. Such enrichment may occur before bisulfite conversion (e.g. customized version of the SureSelect Human Methyl-Seq from Agilent) or after bisulfite conversion (e.g. customized version of the SeqCap Epi CpGiant Enrichment Kit from Roche). Such targeted enrichment can be considered as a further modification/simplification of RRBS (Reduced Representation Bisulfite Sequencing).

As used herein, the term “bisulfite reagent” refers to a reagent comprising in some embodiments bisulfite (or bisulphite), disulfite (or disulphite), hydrogen sulfite (or hydrogen sulphite), or combinations thereof to distinguish between methylated and unmethylated cytidines, e.g., in CpG dinucleotide sequences. Methods of bisulfite conversion/treatment/reaction are known in the art (e.g. WO2005038051). The bisulfite treatment can e.g. be conducted in the presence of denaturing solvents (e.g. in concentrations between 1% and 35% (v/v)) such as but not limited to n-alkylenglycol or diethylene glycol dimethyl ether (DME), or in the presence of dioxane or dioxane derivatives. The bisulfite reaction may be carried out in the presence of scavengers such as but not limited to chromane derivatives. The bisulfite conversion can be carried out at a reaction temperature between 30° C. and 70° C., whereby the temperature may be increased to over 85° C. for short times. The bisulfite treated DNA may be purified prior to the quantification. This may be conducted by any means known in the art, such as but not limited to ultrafiltration, e.g., by means of Microcon columns (Millipore). Bisulfite modifications to DNA may be detected according to methods known in the art, for example, using sequencing or detection probes which are capable of discerning the presence of a cytosine or uracil residue at the CpG site. The choice of specific DNA methylation analysis methods depends on the purpose and nature of the analysis, and is for example outlined in Kurdyukov and Bullock (2016, Biology 5: 3).

The MethyLight assay is a high-throughput quantitative or semi-quantitative methylation assay that utilizes fluorescence-based real-time PCR (e.g., TagMan®) that requires no further manipulations after the PCR step (Eads et al. 2000, Nucleic Acids Res 28:e32). Briefly, the MethyLight process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed in a “biased” reaction, e.g., with PCR primers that overlap known CpG dinucleotides. Sequence discrimination occurs at the level of the amplification process, at the level of the probe detection process, or at both levels. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites or with oligonucleotides covering potential methylation sites.

The EpiTYPER assay involves many steps including gene-specific amplification of bisulfite-converted genomic DNA, in vitro transcription of the amplified DNA, uranil-specific cleavage of transcribed RNA, and MALDI-TOF analysis of the RNA fragments. The EpiTYPER software finally distinguishes between methylated and non-methylated cytosine in the genomic DNA.

Methylation-specific PCR (MSP) refers to the methylation assay as described by Herman et al. 1996 (Proc Natl Acad Sci USA 93:9821-9826), and by U.S. Pat. No. 5,786,146. MSP (methylation-specific PCR) allows for assessing the methylation status of virtually any group of CpG sites within a CpG island, independent of the use of methylation-sensitive restriction enzymes. Briefly, DNA is modified by sodium bisulfite, which converts unmethylated, but not methylated cytosines, to uracil, and the products are subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. MSP primer pairs contain at least one primer that hybridizes to a bisulfite treated CpG dinucleotide. Therefore, the sequence of said primers comprises at least one CpG dinucleotide. MSP primers specific for non-methylated DNA contain a “T” at the position of the C position in the CpG. Variations of MSP include Methylation-sensitive Single Nucleotide Primer Extension (Ms-SNuPE; Gonzalgo & Jones 1997, Nucleic Acids Res 25:2529-2531). Another variation, however including restriction enzyme digestion instead of bisulfite modification as sample pretreatment, is Methylation-Sensitive Arbitrarily-Primed Polymerase Chain Reaction (MS AP-PCR; Gonzalgo et al. 1997, Cancer Research 57:594-599).

Combined Bisulfite Restriction Analysis (COBRA) refers to the methylation assay described by Xiong & Laird 1997 (Nucleic Acids Res 25:2532-2534). COBRA analysis is a quantitative methylation assay useful for determining DNA methylation levels at specific loci in small amounts of genomic DNA. Briefly, restriction enzyme digestion is used to reveal methylation-dependent sequence differences in PCR products of sodium bisulfite-treated DNA. Methylation-dependent sequence differences are first introduced into the genomic DNA by bisulfite treatment. PCR amplification of the bisulfite converted DNA is then performed using primers specific for the CpG islands of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples.

Sanger BS is the original way of analysis of bisulfite-treated DNA: gel electrophoresis-based Sanger sequencing of cloned PCR products from single loci (Frommer et al. 1992, Proc Natl Acad Sci USA 89:1827-1831). A technique such as pyrosequencing is similar to Sanger BS and obviates the need of gel electrophoresis; it, however, requires other specialized equipment (e.g. Pyromark instrument). Sequencing approaches are still applied, especially with the emergence of next-generation sequencing (NGS) platforms. Southern blot analysis of DNA methylation depends on methyl-sensitive restriction enzymes (e.g. Moore 2001, Methods Mol Biol 181:193-201).

Other assays to determine CpG methylation include the HeavyMethyl (HM) assay (Cottrell et al. 2004, Nucleic Acids Res 32, e10; WO2004113567), Methylated CpG Island Amplification (MCA; Toyota et al. 1999, Cancer Res 59:2307-12; WO 00/26401), Reduced Representation Bisulfite Sequencing (RRBS; e.g. Meissner et al. 2005, Nucleic Acids Res 33: 5868-5877), Quantitative Allele-specific Real-time Target and Signal amplification (QuARTS; e.g. WO2012067830), and assays described in Laird et al. 2010 (Nat Rev Genet 11:191-203) and in Kurdyukov & Bullock 2016 (Biology 5(1), pii: E3). Tailored to determine CpG methylation in cfDNA are for instance the cf-RRBS method (De Koker et al. 2019, bioRxiv:663195, doi: http://dx.doi.org/10.1101/663195; WO 2017/162754; Van Paemel et al. 2019, bioRxiv:795047, doi: https://doi.org/10.1101/795047). RRBS methods provide an acceptable balance between genome-wide coverage and accurate quantification of the methylation status and this at an affordable cost. Other methods tailored to analysis of methylation in cfDNA are described in WO2019006269 and US20100240549A1.

Bisulfite reagents convert unmethylated cytosine moieties in DNA into uracil moieties. Drawbacks of such bisulfite reagents are DNA degradation (although perhaps only relevant for long DNA molecules) and lack of complete conversion. Other methods to convert unmethylated cytosine to uracil include TET-assisted bisulfite sequencing (TAB-Seq; involving ten-eleven translocation (TET) enzyme; Yu et al. 2012, Cell 149:1368-1380) and oxidative bisulfite sequencing (oxBS; involving potassium perruthenate; Booth et al. 2012, Science 336:934-937).

An alternative method relies on conversion of 5-methyl-cytosine (5mC) and 5-hydroxy-methyl-cytosine (5hmC) to dihydrouracil (DHU), leaving unmethylated cytosines unaffected. Such method is known as ten-eleven translocation (TET)-assisted pyridine borane sequencing or TAPS. First, 5mC and 5hmC are oxidized by TET enzymes, resulting in conversion to 5-carboxyl-cytosine (5caC). 5caC moieties are then reduced by pyridine borane or 2-picoline borane, resulting in conversion to DHU. Upon duplication or amplification, DHU is converted to thymine (methylated cytosine to thymine conversion) in the duplicated or amplified DNA or RNA. Selective conversion of 5mC (and not 5hmC) to DHU is possible by protecting 5hmC from TET-oxidation by means of adding a glucose to 5hmC (to produce 5gmC) by means of a beta-glucosyltransferase (method referred to as TAPSβ); selective conversion of 5hmC (and not 5mC) is possible by oxidizing 5hmC by means of potassium perruthenate to produce 5-formyl-cytosine (5fmC) and subsequent borane reduction to convert 5fmC to DHU (method referred to as chemical-assisted pyridine borane sequencing or CAPS) (Liu et al. 2019, Nat Biotechnol 37:424-429).

Subject

A “subject”, or “patient”, for the purpose of this invention, relates to any organism such as a vertebrate, particularly any mammal, including both a human and another mammal, e.g., an animal such as a rodent, a rabbit, a cow, a sheep, a horse, a dog, a cat, a lama, a pig, or a non-human primate (e.g., a monkey). In one embodiment, the subject is a human, a rat or a non-human primate. Preferably, the subject is a human. In one embodiment, a subject is a subject with or suspected of having a disease or disorder, or an injury, also designated “patient” herein. In another embodiment, a subject is a subject ready to receive a transplant or allograft, also designated as a “patient eligible for receiving an allograft”. Once an allograft is transplanted in a subject, the subject is a “recipient of the allograft”.

It is to be understood that although particular embodiments, specific configurations as well as materials and/or molecules, have been discussed herein for engineered cells and methods according to the present invention, various changes or modifications in form and detail may be made without departing from the scope of this invention. The following examples are provided to better illustrate particular embodiments, and they should not be considered limiting the application. The application is limited only by the claims.

### EXAMPLES

### Example 1. Age-Related Methylation of CpGs and Correlation with Post-Transplant Kidney Allograft Injury

1.1. Methods

1.1.1. Study Design and Patients

Genome-wide DNA methylation profiling was performed on a cohort of 95 kidney biopsies, obtained prior to kidney transplantation, immediately before implantation: 82 from brain-dead donors and 13 from living donors. Kidney transplants were selected to provide a wide range of donor age, ranging from 16 to 73 years old (average 49±15 years). This implantation cohort was used as a discovery cohort for the association between renal aging and DNA methylation. In addition, a second, independent cohort of 67 kidney transplant biopsies was selected to validate the findings from the discovery cohort: 58 from brain-dead donors and 9 from living donors. These validation-set biopsies were obtained immediately after implantation and reperfusion during the transplant procedure. Also here, donor age ranged widely from 16 to 79 years old (average 49±16 years). All transplant biopsies were selected from our Biobank, where biopsies are performed at implantation, post-reperfusion, 3, 12 and 24 months after transplant in each kidney transplant recipient at the University Hospitals Leuven (Naesens et al. 2015, J Am Soc Nephrol 27:281-292). No left and right kidney transplants from the same donor were included. Immunosuppressive therapy consisted of tacrolimus, mycophenolate mofetil and corticosteroids tapering. Based on results of protocol-specified transplant biopsies at 3 months post-transplant, corticosteroids are discontinued or continued at a low dose. No biopsies for cause (“indication biopsies”) performed at the time of transplant dysfunction, were included in this study. All transplant recipients gave written informed consent as part of this Biobank, which was approved by the local ethical committee (553364). The biopsies from brain-dead donor kidneys were also profiled for our previous study on ischemia-associated DNA methylation changes during kidney transplantation (Heylen et al. 2018, J Am Soc Nephrol 29:1566-1576).

1.1.2. Epigenome-Wide Analyses

Genomic DNA was extracted from all biopsies using Allprep DNA/RNA/miRNA Universal kit (Qiagen, Hilden, Germany). For genome-wide methylation analysis, DNA was bisulphite converted using EZ DNA Methylation kit (Zymo Research, Irvine, Calif., USA) and subsequently probed for DNA methylation levels using the Infinium MethylationEPIC Beadchips (Illumina, San Diego, Calif., USA). These chips target methylation at single-nucleotide resolution at around 850 000 CpG sites across the genome, covering 99% of genes in the Reference Sequence database (Pidsley et al. 2016, Genome Biol 17:208). For the validation cohort, Infinium HumanMethylation450 arrays (Illumina, San Diego, Calif., USA) were used, that target methylation at single-nucleotide resolution at around 450 000 CpG sites across the genome. Quality control consisted of: removal of probes for which any sample did not pass a 0.01 detection P-value threshold, bead cut-off of 0.05, and removal of probes on sex chromosomes. Raw data were normalised using BMIQ using the ChAMP pipeline (Morris et al. 2014, Bioinformatics 30:428-430), and batch corrected using Combat embedded in the ChAMP pipeline. In addition, batch effect was prevented by distributing samples of different ages among all batches. Methylation levels (beta-values) were logarithmically transformed to M-values for all statistical tests. Coefficients in the graphs are based on beta-values to permit its interpretation.

1.1.3. Clinical and Histological Data

Clinical data of both donors and recipients were collected in electronic clinical patient charts. Post-transplant data were collected during routine clinical follow-up of the transplant recipients. Transplant biopsies were scored by one pathologist (EL) according to the revised Banff criteria (Sis et al. 2010, Am J Transplant 10:464-471). For this study, we focused on the typical age-associated lesions, at the time of implantation, as well as at one year after transplant: interstitial fibrosis (Banff “ci” score), tubular atrophy (Banff “ct” score), intimal thickening (Banff “cv” score), and glomerulosclerosis. For the latter, the total number of glomeruli in each biopsy, and the number of globally sclerosed glomeruli, were calculated separately. Only biopsies with >10 glomeruli (A quality) were included for evaluation of glomerulosclerosis. 41.1% of deceased renal transplant biopsies had some degree of interstitial fibrosis at the time of transplant. At one year after transplant, this number increased to 62.7% (ci1 42.4%, ci2 15.3%, ci3 5%). Tubular atrophy prevalence increased from 58.6% to 94.9% after one year (ct1 83.1%, ct2 11.8%). Glomerulosclerosis was present in 41.2% of biopsies at the time of transplant, and 51.7% of biopsies after one year (41.4% gs1, 10.3% gs2). Arteriosclerosis prevalence increased from 16.2% to 62.7% at one year after transplant (cv1 33.9%, cv2 25.4%, cv3 3.4%).

1.1.4. Statistical Analyses

All statistical analyses were performed using RStudio (version 0.99). The effect of age on DNA methylation was examined for all CpGs individually using linear regression adjusted for donor gender, cold ischemia time and type of donation (deceased versus living). Since only 2 out of 95 donors from the implantation cohort had diabetes mellitus and none of them had glomerulosclerosis at baseline, we did not correct for donor diabetes. For this, we used the CpGassoc package for R (Barfield et al. 2012, Bioinformatics 28:1280-1281). For the postreperfusion cohort, also anastomotic warm ischemia time was included in the multivariable model, as these biopsies experienced additional ischemia during implantation. Results were corrected for multiple testing by Benjamini-Hochberg correction, and a false discovery rate (FDR)<5% was considered as significant. Hyper- versus hypomethylation events were compared using binomial tests. Based on the CpG-site specific results, we searched for significantly differentially methylated regions upon age (consisting of several CpG sites associated with age), by combining p-values from nearby sites, using the comb-p pipeline (Pedersen et al. 2012, Bioinformatics 28:2986-2988). Differentially methylated regions were considered significant when their P-value adjusted for multiple testing correction (Šidák correction) was below 0.05. Regions were considered to be hypermethylated, respectively hypomethylated upon age when at least 70% of their CpG sites were hypermethylated, respectively hypomethylated with age. Differentially methylated regions were annotated according to genes based on overlap using the Ensembl genome database (GRCh37). Promoters were defined as regions starting 1500 base pairs before the transcription start site and ending 500 base pairs after. Pathway analysis was performed using Ingenuity Pathway Analysis (IPA). As too many differentially methylated regions were significant using the FDR 0.05 threshold to enable Ingenuity Pathway Analysis, a threshold of 0.0001 was used. To assess whether CpG sites measured on the methylation arrays are not biased towards genes involved in age-related processes, we performed additional Ingenuity Pathway Analyses by assigning a p-value of 0.01 and 1 to all differentially methylated regions that we detected. However, in none of these analyses age-related pathways were ranked high (in the top 10).

The DNA methylation level of all age-associated CpGs were individually correlated to the histology scores and to reduced allograft function (defined as an estimated glomerular filtration rate (eGFR) below 45 mg/ml/1.73 m² calculated by the MDRD formula (Poggio et al. 2006, Am J Transplant 6:100-108) using linear and logistic regression, respectively, adjusted for donor gender.

We also investigated whether the DNA methylation changes upon aging occurred preferentially in genes associated with a specific functional anatomical unit of the kidney. For the glomerulus, we used the human renal glomerulus-enriched gene expression dataset published by Lindenmeyer et al, which is based on microarray analysis of microdissected glomeruli and tubulointerstitial specimen (Lindenmeyer et al. 2010, PloS one 5:e11545). The authors did not publish the tubulointerstitial geneset, and no other study on the transcriptome of microdissected human kidneys was found. Therefore, we used the GUDMAP database, defining the markers of the renal proximal tubules and the renal interstitium, respectively. The human homologue genes of the described mouse markers were used.

1.2. Results

1.2.1. Genome-Wide Changes in DNA Methylation Upon Ageing

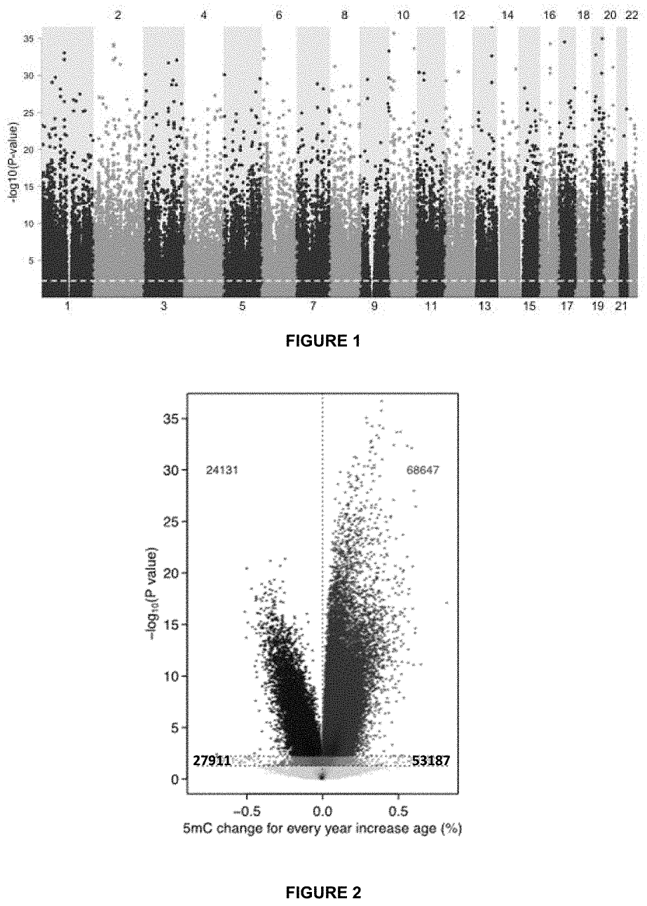

To investigate DNA methylation changes at the genome-wide level in the kidney, we profiled 95 renal biopsies obtained prior to kidney transplantation. We hereafter refer to this cohort of 95 biopsies as the implantation cohort. Donor age ranged from 16 to 73 years (49±15), 49 (60%) donors were male and 13 (14%) were living donors. We used Infinium MethylationEPIC Beadchips (Illumina, San Diego, Calif., USA) to measure DNA methylation of ˜850 000 CpG sites across the genome, covering 99% of genes in the Reference Sequence database (Pidsley et al. 2016, Genome Biol 17:208). After quality control, normalization and batch correction, we correlated age with DNA methylation for each individual CpG using linear regression adjusted for donor gender, cold ischemia time and donor type (deceased versus living). This revealed a significant linear association (FDR<0.05) between donor age and the extent of methylation for 92 778 out of 803 663 CpG sites (11.5%). The top 50 from these 92 778 CpG sites is represented in Table 1. A Manhattan plot of the 92 778 sites shows how they were distributed throughout the genome with significance levels up to 2.38×10⁻³⁷ (FIG. 1).

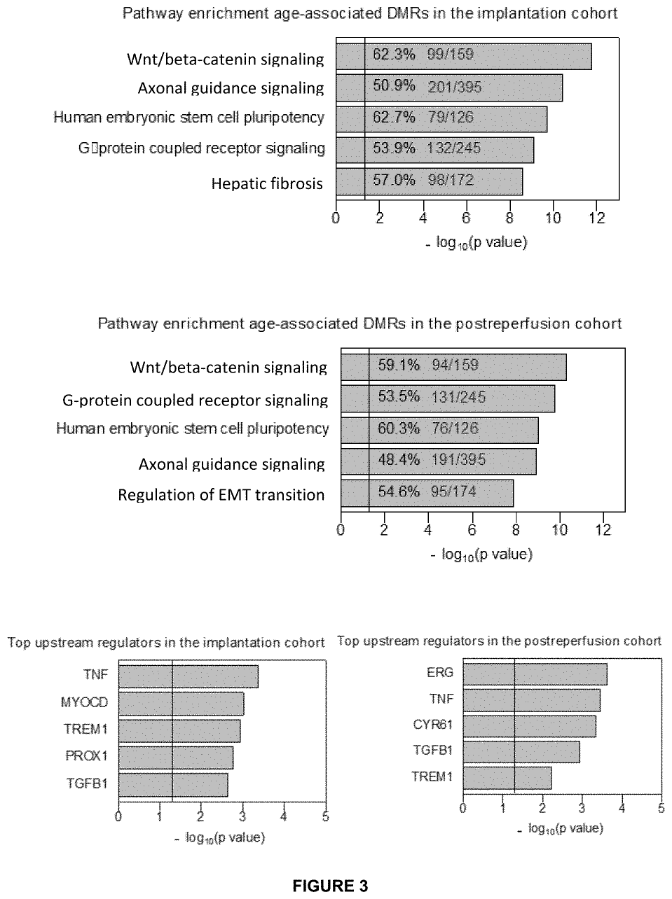

Of the 92 778 CpG sites, significantly more CpG sites were hypermethylated with increasing donor age: 68 647 (74.0%) hypermethylated versus 24 131 (26.0%) hypomethylated CpG sites (binomial test P<1×10⁻¹⁵) (FIG. 2). Per decade increase in donor age, DNA methylation increased by 0.9% for hypermethylated regions, but decreased by 1.1% for hypomethylated regions. For CpGs located inside gene promoters (24 267 or 26.2% of the CpGs), this deviation towards age-associated hypermethylation was even more pronounced, with 20 270 (83.5%) CpGs being hypermethylated and 3 997 (16.5%) being hypomethylated (binomial test P<1×10⁻¹⁵). The shift towards hypermethylation in gene promoters is consistent with the epigenetic drift model proposed in previous studies on other tissues (Jones et al. 2015, Aging Cell 14:924-932). Although less striking, there was still a trend towards hypermethylation upon aging outside the CpG island context, with 25 542 of 43 648 CpGs in open sea context (58.5%) showing hypermethylation.

1.2.2. Loss of DNA Hydroxymethylation Triggers Age-Associated Hypermethylation

DNA demethylation is initiated by ten-eleven translocation (TET) enzymes that convert 5-methylcytosine (5mC) to 5-hydroxymethylcytosine (5hmC) (Williams et al. 2011, Nature 473:343-348). These enzymes are ubiquitously expressed in adult cells, including the kidney where 5hmC is particularly abundant (Bachman et al. 2014, Nature Chem 6:1049-1055). To determine whether age-related kidney hypermethylation is perhaps due to a decrease in DNA demethylation, we profiled 5hmC genome-wide in 6 renal biopsies of the implantation cohort that were also profiled for methylation. We selected 3 biopsies from donors aged 25 years or less, and 3 biopsies from donors aged 65 years or more. Most sites hypermethylated in old versus young kidneys (P<0.05) exhibited a decrease in DNA hydroxymethylation (7 290 of 7 809 sites, 93.4%), suggesting that reduced DNA demethylation underlies the increase in DNA methylation in aged kidneys. To assess whether this decrease in DNA hydroxymethylation upon aging was due to reduced TET expression, we determined TET1, TET2 and TET3 transcription in deceased donor biopsies prior to transplantation. There was however no correlation between donor age and TET1, TET2, or TET3 gene expression (P>0.05 for each correlation). Donor age also did not correlate with expression of any of the DNA methylating enzymes (DNMT1, DNMT3A and DNMT3B) (P>0.05 for each correlation).

1.2.3. Ageing and DNA Hypermethylation of Wnt-Signaling Pathway Genes

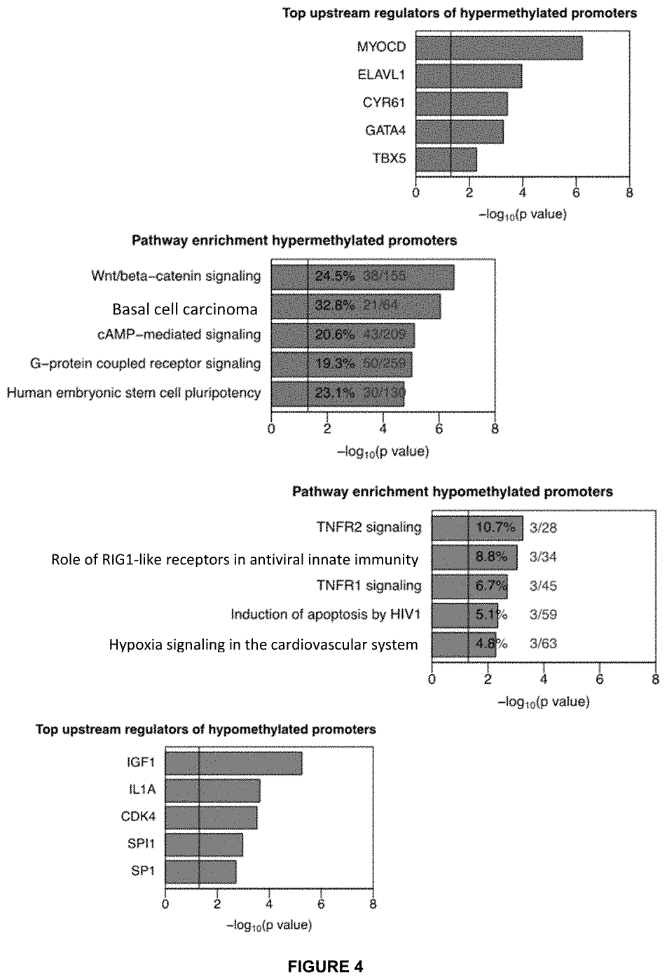

To determine which genes were predominantly affected by methylation changes upon renal aging, we assessed the 92 778 CpGs as differentially methylated regions (DMRs), whereby a DMR was defined as nearby located CpGs demonstrating the same age-associated methylation changes while adjusting for donor gender, type of donation and cold ischemia time. Overall, 57 343 regions were differentially methylated upon aging, of which 10 285 surpassed a Šidák multiple testing corrected P-value of 0.05, with 5 445 highly significant DMRs surpassing a Šidák multiple testing corrected P-value of 0.0001. The top 99 from these 5 445 DMRs is represented in Table 2. When assigning these 5 445 highly significant DMRs to an individual gene and verifying whether they were enriched in specific pathways, we found that the top-enriched canonical pathway was the Wnt/beta-catenin signaling pathway (P=1.8×10⁻¹²; 62.3% overlap), which is involved in cellular proliferation and renal fibrosis (FIG. 3) (Edeling et al. 2016, Nat Rev Nephrol 12: 426-439). We also eliminated the possibility that enrichment for the Wnt/catenin pathway was the result from a bias in the CpGs selected on the arrays (see methods).

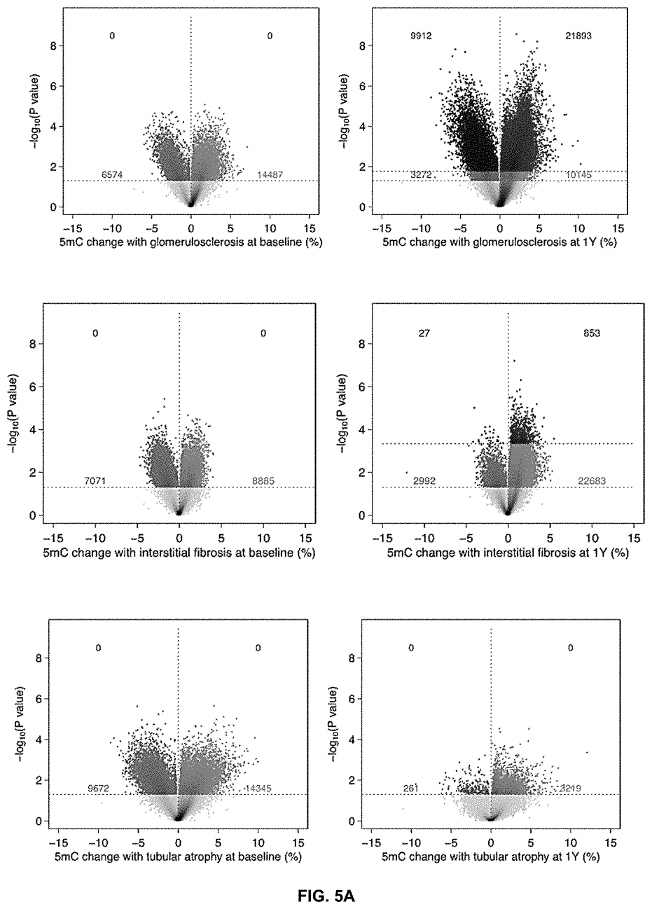

As DNA methylation changes affecting gene promoters are often associated with gene expression changes (with hypermethylation reducing, and hypomethylation inducing gene expression), we specifically analyzed genes with a hyper- or hypomethylated region in their promoter (2 721 hypermethylated regions inside promoters versus 251 hypomethylated regions). Pathway analysis of the genes with a hypermethylated promoter (n=2 570, not shown) revealed that the Wnt-/beta-catenin signaling pathway, cAMP mediated signaling, G-protein coupled receptor signaling and embryonic stem cell pluripotency were among the top enriched pathways (FIG. 4). Of the 38 Wnt-/beta-catenin signaling pathway genes with a hypermethylated region in their promoter, 18 are considered inhibitory, i.e. counteracting the Wnt-/beta-catenin pathway, including the dickkopf Wnt signaling inhibitors (DKK), several SOX transcription factors, Wnt inhibitory factor 1 (WIF1), secreted frizzled related protein 2 (SFRP2), and retinoic acid receptor alfa and beta (RARA and RARB).

In contrast, genes with hypomethylated promoters (n=162, not shown) were enriched for inflammatory and immunological pathways, such as TNFR2 signaling and TNTR1 signaling (including the genes: TNF receptor associated factor 2 (TRAF2), NFKB inhibitor epsilon (NFKBIE), and TRAF family member associated NFKB activator (TANK)), and hypoxia signaling and induction of apoptosis (FIG. 4). Other, less enriched pathways include the Th1 pathway (P=5.83×10⁻³; 3.1% overlap), death receptor signaling (P=1.29×10⁻²; 3.4% overlap), IL17A signaling in fibroblasts (P=1.65×10⁻²; 5.7% overlap), Th1 and Th2 activation pathway (P=1.79×10⁻²; 2.2% overlap), IL-6 pathway (P=3.13×10⁻²; 2.5% overlap) and autophagy (P=3.21×10⁻²; 4.0% overlap). Interestingly, the top upstream regulator of genes with hypomethylated regions in their promoter was insulin-like growth factor-1 (IGF1) (P<0.001) (FIG. 4), a key regulator of longevity and aging (Russell et al. 2007, Nat Rev Mol Cell Biol 8: 681-691).

To independently confirm these observations, we associated DNA methylation with donor age in an independent validation cohort of 67 kidney biopsies obtained after reperfusion (post-reperfusion cohort). Mean donor age in this cohort was 49±16 years, 41 (61.2%) donors were male and 9 (13.4%) biopsies were from living donors. In this cohort, methylation levels of 64 336 CpGs (out of 435 162 (14.8%) CpGs profiled by Infinium 450K arrays) were independently associated with age at FDR<0.05. Again, older age induced more hyper-than hypomethylation (57 236 (90.0%) versus 7 100 (10.0%); Chi-square test P<1×10⁻¹⁵)), and the top enriched pathway among genes with a DMR upon aging (multiple testing corrected P<0.0001) was the Wnt/beta-catenin pathway (FIG. 3), demonstrating the robustness of these findings.

1.2.4. Role of Age-Associated DNA Hypermethylation in Nephrosclerosis

Next, we investigated whether age-associated DNA methylation changes correlated with any of the structural changes that are characteristic for renal aging. For this, we selected donor kidneys from deceased patients from the implantation cohort (n=82). We focused on the histological characteristics of the implantation biopsies as well as the protocol biopsies at one year after transplant (i.e. at the time of stable kidney transplant function). Biopsies for cause were not included, to eliminate any potential bias because of graft rejection.

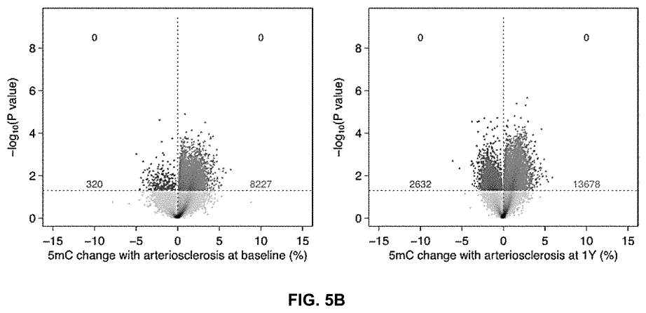

Since the prevalence of tubular atrophy, arteriosclerosis, interstitial fibrosis and glomerulosclerosis in kidney biopsies obtained prior to transplantation increases with age, one would expect that age-associated DNA methylation also correlates with these histological characteristics at transplantation. However, none of the 92 778 CpG sites whose methylation status correlated with age was also correlated with these lesions at the time of transplantation (FDR>0.05 for all comparisons). Intriguingly, however, 31 805 out of 92 778 CpG sites (34.3%) correlated with glomerulosclerosis (at FDR<0.05) (top 50 from these 31 805 is represented in Table 3), and 880 out of 92 778 (0.9%) CpG sites correlated to a lesser extent with interstitial fibrosis (at FDR<0.05) (top 50 from these 880 is represented in Table 4) at one year after transplantation. In contrast, none of the CpGs were associated with future tubular atrophy or arteriosclerosis at FDR<0.05 (FIGS. 5A-5B). This suggests that age-associated methylation correlated strongly with future but not present glomerulosclerosis.

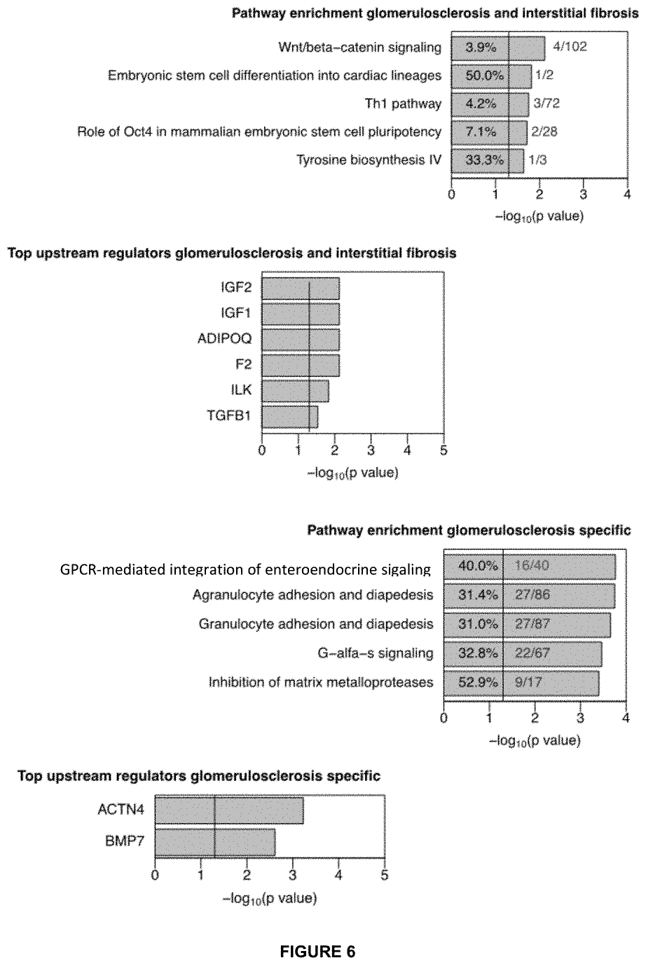

Next, we explored which pathways were affected by the methylation changes that associated with both age and with interstitial fibrosis and/or glomerulosclerosis. Genes whose age-associated promoter methylation uniquely correlated with glomerulosclerosis (n=5517) were enriched in immunological and matrix metalloproteases inhibition pathways, with actinin alpha 4 (ACTN4) and bone morphogenic protein 7 (BMP7) as top upstream regulators (FIG. 6). Too few genes were uniquely associated with interstitial fibrosis to enable pathway enrichment analysis. For 293 genes with age-dependent methylation, methylation inside the promoter correlated with both future interstitial fibrosis as well as glomerulosclerosis at FDR<0.05 These genes were again enriched for members of the Wnt/beta-catenin signaling pathway, with IGF1 and IGF2 as top upstream regulators (FIG. 6). Thus, age-dependent epigenetic changes in the Wnt/beta-catenin signaling pathway are involved in both interstitial fibrosis and glomerulosclerosis, and not unique to these lesions individually.

1.2.5. Age-Associated DNA Methylation Affects Genes Involved in Nephrosclerosis

Since age-associated methylation changes predominantly associated with glomerulosclerosis, we evaluated whether affected genes were indeed expressed in the glomerular compartment. We focused on genes with high expression in the glomerulus relative to the tubulo-interstitium, as assessed by Lindenmeyer et al. 2010 (PloS One 5:e11545). Out of the 617 glomerular-specific genes for which DNA methylation in the gene promoter was assessed, 138 genes (22.4%) exhibited a differentially methylated promoter region with increasing age (FDR<0.05). This was significantly higher than expected based on random chance (4 621/41 780 (11.1%); chi square P<0.001). Because the age-associated epigenetic changes also correlated with interstitial fibrosis at one year after transplantation, we additionally evaluated whether typical renal interstitium markers were enriched for age-associated methylation changes. Of 34 interstitial markers defined by the GenitoUrinary Development Molecular Anatomy Project (GUDMAP), there were 9 genes for which the promoter was differentially methylated upon aging (26.5%), which is also significantly more than expected by chance (4 621/43 157 (11.1%); chi square P<0.001). In line with the lack of correlation between age-associated methylation changes and tubular atrophy, none of the 31 tubular marker genes defined by GUDMAP contained a differentially methylated promoter upon aging.

1.2.6. Role of Age-Associated DNA Methylation in Post-Transplant Function

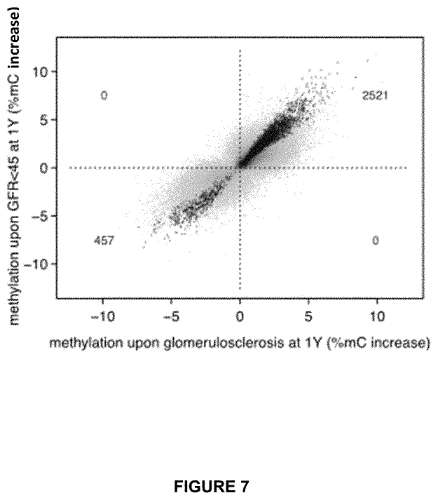

Finally, we assessed whether age-associated methylation changes also correlated with renal function at one year after transplantation (n=82). Out of 92 778 CpG sites whose methylation changed upon increased age, 6 188 sites (6.7%) also correlated with reduced renal transplant function (eGFR<45 ml/min/1.73 m²) at one year after transplantation (FDR<0.05). Age-associated CpG sites that correlated with glomerulosclerosis at one year after transplantation (n=31 805) were more frequently associated with reduced allograft function than those that did not correlate with glomerulosclerosis (2 978/31 805 (9.4%) versus 3 210/60 973 (5.3%), chi square P<0.001). Strikingly, we observed that 2 521 out of 2 978 sites were both correlated with glomerulosclerosis and reduced renal allograft function. A similar observation was done for 457 hypomethylated CpG sites (FIG. 7).

1.2.7. Discussion

This study provides the first kidney-specific study of age-associated epigenetic alterations. Interestingly, aging affected predominantly the methylation of genes whose cellular functions are known to be involved in aging processes of the kidney, suggesting a causal relation between DNA hypermethylation and age-associated kidney dysfunction. Indeed, these methylation changes correlated with future glomerulosclerosis and interstitial fibrosis, as well as with reduced renal function after transplant. In addition, we demonstrated for the first time that age-associated hypermethylation in kidneys is accompanied by loss of DNA hydroxymethylation, suggesting that reduced activity of the TET demethylation enzymes drives these changes.

The observed DNA methylation changes in the aging kidney were quite substantial, as 11.5% of the CpG sites assessed were significantly altered, which is much more than the previously described 0.05 to 4% of CpG sites previously described for other organs (Bacos et al. 2016, Nat Commun 7:11089; Hernandez et al. 2011, Hum Mol Genet 20:1164-1172). This difference can possibly be attributed to the fact that kidney cells are differentiated and generally non-proliferative, which enables the progressive accumulation of these epigenetic changes. Most of the observed changes involved DNA hypermethylation, not only in gene promoters and CpG islands, but also outside of these regions. This contrasts with studies in other tissues where CpG sites outside of gene promoters and CpG islands exhibited profound DNA demethylation (Jones et al. 2015, Aging Cell 14:924-932). Interestingly, this age-induced hypermethylation was accompanied by loss of DNA hydroxymethylation, suggesting that reduced activity of the TET demethylation enzymes drives these changes. Interestingly, TET and DNMT expression did not correlate with age, which suggests that other factors contribute to the reduction in DNA hydroxymethylation. Possibly, reduced TET activity could be attributed to increased oxidative stress of the aged kidney, which is known to inhibit TET activity (Hommos et al. 2017, J Am Soc Nephrol 28: 2838-2844). Such hypothesis is consistent with our previous study, in which we show that oxygen shortage during ischemia also reduces TET activity and subsequent hydroxymethylation, leading to increased DNA methylation of the kidney during kidney transplantation (Heylen et al. 2018, J Am Soc Nephrol 29:1566-1576). The effects of aging that we describe here could, however, not be attributed to cold ischemia time, as all of our statistical analyses were adjusted for cold ischemia time or the type of donation (as living donor kidneys are characterized by very little ischemia compared to deceased donors), indicating that the effect of aging on DNA methylation is independent of ischemia. Overall, this suggests that we are the first to couple age-associated increases in DNA methylation to decreased hydroxymethylation. Interestingly, apart from the brain, the kidney is characterized by the highest levels of hydroxymethylation across organs (Bachman et al. 2014, Nat Chem 6:1049-1055). These high levels of 5-hydroxymethylation might render the kidney more prone to DNA hypermethylation upon reduced TET activity. The kidney therefore also represents a unique organ to study methylation-associated aging processes.

Several studies have described DNA methylation changes upon aging in various organs (Hannum et al. 2013, Mol Cell 49:359-367; Horvath 2013, Genome Biol 14:R115), but until now it has remained elusive which genes are affected and whether this has functional implications in these organs (Sen et al. 2016, Cell 166:822-839). Interestingly, the cellular functions that are affected by aging in the kidney, such as decreased epithelial cell proliferation, increased susceptibility to apoptosis, deteriorated stem cell function and activation of inflammatory cells (Schmitt & Cantley 2008, Am J Physiol-Renal Physiol 294:F1265-F1272), were all enriched in the pathways that we observed to be affected by methylation upon aging. This suggests that age-associated epigenetic changes causally underlie the age-associated functional changes. Interestingly, age-associated hypermethylation of gene promoters was most strongly observed in genes involved in the Wnt-catenin signaling pathway. It is well-established that activation of this pathway in aging mice leads to reduced progenitor cell activation and increased fibrosis (Liu et al. 2007, Science 317:803-806; Brack et al. 2007, Science 317:807-810). Hypermethylation of this pathway upon aging, associated with reduced gene expression, seems to be in contrast with the age-associated activation of this pathway. However, many of these hypermethylated genes are inhibitors of this pathway, or downregulated upon pathway activation. These include several dickkopf Wnt signaling inhibitors (DKK), SOX transcription factors, Wnt inhibitory factor 1 (WIF1), secreted frizzled related protein 2 (SFRP2), and retinoic acid receptor alfa and beta (RARA and RARB). SOX transcription factors are also involved in the regulation of embryonic development and cell fate. Moreover, inhibition of SOX2 has been linked to activation of apoptosis. Hypermethylation also preferentially occurred in genes involved in stem cell pluripotency, such as BM P7, several frizzled class receptors, and transcription factors such SOX2 and TCF3.

We also observed that the age-associated DNA methylation changes did not correlate with the severity of structural lesions in the biopsy collected at the time of kidney transplantation. This is striking, as DNA methylation profiles are highly dependent on the cell type. In contrast, there was a profound correlation of age-associated epigenetic changes to future injury after transplant, more specifically to glomerulosclerosis and to a lesser extent interstitial fibrosis, while no correlation was observed with tubular atrophy and arteriosclerosis. In line with these findings, epigenetic aging also preferentially occurred in genes involved in glomerular function and interstitium development. Thus, while aged kidneys are characterized by glomerulosclerosis, tubular atrophy, interstitial fibrosis and arteriosclerosis, our results suggest that the molecular mechanisms driving these changes differ. This is in line with our previous study where we demonstrated that telomere attrition, another mechanisms of senescence, was associated with renal arteriosclerosis, but not with other age-associated histological findings (De Vusser et al. 2015, Aging 7:766-775). Thus, not all hallmarks of aging, such as replicative senescence, klotho deficiency, inflammation, autophagy, and oxidative stress (O'Sullivan et al. 2017, J Am Soc Nephrol 28:407-420), evoke similar structural and functional changes in the kidney. Strategies to combat the impact of renal aging will therefore most likely need to target different pathophysiological processes.

Our results demonstrate that age-associated DNA methylation changes are mainly involved in age-associated fibrogenesis, both in the interstitium as well as in the glomerulus. Indeed, both lesions are fibrotic events, characterized by similar cellular changes, involving the loss of epithelial cells and their vascular capillary bed, and the accumulation of activated myofibroblasts, matrix, and inflammatory cells (Edeling et al. 2016, Nat Rev Nephrol 12:426-439; Liu 2011, Nat Rev Nephrol 7:684-696). Since epigenetics, and more specifically DNA methylation alterations, can determine long-term cellular phenotype changes that are transmitted during cell division (Petronis 2010, Nature 465:721-727; Portela et al. 2010, Nat Biotech 28:1057-1068), it is not surprising that these changes are involved in the phenotype switch that occurs in cells upon fibrogenesis.

Our findings are in line with studies on a rodent model of folic-acid induced kidney fibrosis, where methylation changes were shown to drive kidney fibrosis and also preferentially affected genes in the Wnt/beta catenin-signaling pathway (Bechtel et al. 2010, Nat Med 16: 544-550). Several animal studies also demonstrated that the Wnt/beta-catenin pathway plays an important role in interstitial fibrosis, glomerulosclerosis and chronic allograft injury (Edeling et al. 2016, Nat Rev Nephrol 12:426-439; von Toerne et al. 2009, Am J Transplant 9:2223-2239; Dai et al. 2009, J Am Soc Nephrol 20: 1997-2008; Zhou et al. 2017, J Am Soc Nephrol 28: 2322-2336; Zhou et al. 2012, Kidney Int 82: 537-547). Moreover, DKK1 and DKK2, inhibitors of the Wnt pathway, are reduced in expression in murine renal fibrosis models (Edeling et al. 2016, Nat Rev Nephrol 12:426-439; He et al. 2009, J Am Soc Nephrol 20:765-776) and these genes were hypermethylated upon aging in our study. The observation that age-associated epigenetic changes correlate more with future fibrosis, than with the injury already apparent at the time of measurement is, however, remarkable. This might suggest that these DNA methylation changes upon aging prime the kidney for increased vulnerability to injury during and after transplantation, and could act as some sort of susceptibility factor. This is also consistent with older donor kidneys being more susceptible to ischemic injury (Tullius et al. 2000, J Am Soc Nephrol 11:1317-1324).

For the field of transplantation, these observations are relevant, as interstitial fibrosis and tubular atrophy are generally considered as one entity (interstitial fibrosis/tubular atrophy) (Solez et al. 2008, Am J Transplant 8:753-760). Our results suggest, however, that although both can share a common cause, DNA methylation changes play a role in the development of interstitial fibrosis, but not of tubular atrophy. Our patient-based study however does not enable us to assess whether age-associated DNA methylation changes really drive these functional changes or are merely reflecting them. Another limitation is that post-transplant histology can be influenced by several donor, recipient and post-transplant factors. We accounted for several of these in this study, for example by excluding biopsies for cause (i.e. biopsies performed at the time of graft dysfunction) or by adjusting our analyses for type of donation, donor gender and cold ischemia time. Moreover, it is very unlikely that diabetes mellitus of the donor confounded the association with glomerulosclerosis, since only 2 out of 95 donors from the implantation cohort had diabetes mellitus and none of them had glomerulosclerosis at baseline. Because many of the potential confounding variables often occur at low frequency, it was statistically not possible to account for all of them when assessing the role of DNA hypermethylation for transplant outcome. Larger studies that also adjust for these post-transplant parameters will be needed to confirm our observations. Finally, future work is also needed to build a model based on age-induced DNA methylation CpG sites that can reliably predict outcome of glomerulosclerosis, interstitial fibrosis, graft function or survival.

In conclusion, this study opens new perspectives to combat the consequences of aging in the kidney. As DNA methylation is reversible and targeted modification of DNA methylation recently have become feasible (Liu et al. 2016, Cell 167:233-247), it is at least theoretically possible to start modifying epigenetic information during kidney preservation as a potential approach to slow nephrosclerosis and prolong transplant survival.

### Example 2. Lschemia-Induced Methylation of CpGs and Correlation with Post-Transplant Kidney Allograft Injury

2.1. DNA Hypermethylation of Kidney Allografts Following Ischemia.

To evaluate DNA methylation changes arising during cold ischemia, a prospective clinical study was set up to collect paired pre-ischemic procurement and post-ischemic reperfusion biopsies of 13 brain-dead donor kidney transplants (Heylen et al. 2018, J Am Soc Nephrol 29:1566-1576; PCT/EP2018/086509). This paired design minimized inter-individual differences, such as genetic differences, age and gender, which are known to profoundly influence DNA methylation levels. The average cold ischemia time was 10.1±4.1 hours.

DNA methylation levels were analysed for >850,000 CpGs using Illumina EPIC beadchips micro-arrays (Pidsley et al. 2016, Genome Biol 17: 185-192) and, following normalisation, pre- versus post-ischemia levels were compared in a pair-wise fashion. First, global DNA methylation levels averaged across all probes were evaluated. An increase in each transplant pair following ischemia was observed (median increase: 1.3±0.9%, P=0.0002). Next, it was assessed which individual CpGs were affected by ischemia. Identified were 91,430 differentially methylated sites (P<0.05), most of which showed hypermethylation in the post-reperfusion biopsy (82,033 CpG sites, 90%; P<0.00001). Methylation levels of these CpGs increased up to 12.1% after ischemia. Significantly hypermethylated CpGs were frequently found near CpG islands, particularly within CpG island shores (20.2% versus 17.8% by random chance, P<0.00001). We therefore grouped methylation of individual CpGs per CpG island: the vast majority of CpG islands (22,001 out of 26,046, 84.5%) were hypermethylated after ischemia, of which 8,018 at P<0.05. When correcting for multiple testing (FDR<0.05), 4,156 out of 26,046 islands analysed (16.0%) were differentially methylated, 4,138 (99.6%) of which showed hypermethylation after ischemia. These islands corresponded to 2,388 unique genes. Interestingly, the CpG island with the highest increase in methylation was located in the DDR1 promoter, a gene known to be involved in apoptosis and kidney fibrosis (Borza 2014, Matrix Biol 34:185-192).

2.2. Dose-Dependency of Ischemia-Induced DNA Methylation Changes.

Each additional hour of cold ischemia time increases the risk of developing chronic allograft failure (Debout et al. 2015, Kidney Int 87: 343-349). Therefore, we assessed whether a similar correlation exists between cold ischemia time and the extent to which ischemia-induced methylation changes occur. We assembled a second independent cross-sectional cohort of 82 post-ischemic pre-implantation biopsies. In pre-implantation biopsies DNA methylation levels cannot be affected by warm ischemia nor reperfusion, and therefore cell composition changes cannot occur, excluding the possibility that changes in cell type composition underlie the methylation changes.

Cold ischemia time ranged from 4.7 to 26.7 hours. Genome-wide DNA methylation levels analysed using Illumina EPIC beadchips were correlated with cold ischemia time using a linear regression adjusted for donor gender and age. Methylation levels correlated with cold ischemia time for 29,700 CpG sites (P<0.05), the bulk of these (21,413 CpGs, 72.1%) showing ischemia-time dependent hypermethylation (P<0.00001). In some CpGs, methylation increased up to 2.6% with each hour increase in cold ischemia time. These CpGs were also more likely to be hypermethylated in the post-ischemic biopsies analysed in the longitudinal cohort (P<0.0001). Particularly, up to 2,932 CpGs were hypermethylated in both cohorts (P<0.05) and mainly affected CpG islands and shores, and less frequently shelves and open sea regions. When classifying these 2,932 CpGs based on kidney chromatin state, these CpGs were predominantly found at enhancers and gene promoters.

At the CpG island level, cold ischemia time significantly correlated with methylation levels of 189 CpG islands (FDR<0.05, adjusted for age and gender). The vast majority of these were hypermethylated (156 islands, 82.5%, FIG. 4D). Of these 156 CpG islands, 66 (42.3%) were also hypermethylated at an FDR<0.05 threshold in the longitudinal cohort (versus 15.9% expected by random chance; P<0.00001). We thus identified 66 CpG islands (listed in Table 5; for listing of the CpG sites within these islands: see Table 2 of PCT/EP2018/086509) that were consistently hypermethylated at a stringent multiple correction threshold in both cohorts.

2.3. Ischemia-Induced Hypermethylation and Chronic Allograft Injury.

Next, we assessed whether these methylation changes become transient or stably imbedded in the kidney methylome after the ischemic insult. We measured DNA methylation in biopsies obtained several months after transplantation (longitudinal cohort) and assessed hypermethylation in the 66 CpG islands. Interestingly, we observed that CpGs located in these islands were still hypermethylated at 3 months and 1 year after transplantation.

We then investigated whether ischemia-induced hypermethylation observed at the time of transplantation correlates with chronic allograft injury (calculated by the Chronic Allograft Damage Index (CADI) score; Yilmaz et al. 2003, J Am Soc Nephrol 14:773-779). When correlating the methylation status of 1 634 CpGs in the 66 islands with injury, we found that 487 (30%) and 332 (20%) CpGs were positively correlated with CADI score at 3 months, respectively at P<0.05 and FDR<0.05, whereas 402 (25%) and 135 (8%) CpGs were associated with CADI at 1 year. This was significantly more than the 48 and 14 CpGs negatively correlating (P<0.05) with CADI at 3 months and 1 year, respectively. When adjusting for donor age and gender, similar effects were observed. The bias towards a direct correlation between hypermethylation and future injury was also not detected at baseline injury, as only 43 out of 75 (57%; P>0.05) CpGs correlated positively with CADI at baseline. Also when adjusting for cold and warm ischemia time, DNA methylation correlated better with future injury than with injury already evident at the time of transplantation.

2.4. DNA Hypermethylation Predicts Chronic Allograft Injury.

Having shown that ischemia-induced hypermethylation of kidney transplants correlates with chronic allograft injury, we tested whether a methylation-based risk score at the time of transplantation could predict chronic injury 1 year after transplantation. The latter was defined by a CADI>2, representing a threshold that predicts graft survival at 1 year after transplantation. First, we developed a risk score reflecting DNA methylation in the 66 CpG islands (Table 5) weighted for their correlation with chronic injury at one year after transplant in the pre-implantation cohort. Patients with a methylation risk score (MRS) in the highest tertile had an increased risk (odds ratio [OR], 45; 95% confidence interval [95% CI], 8 to 499; P<0.00001) to develop chronic injury relative to patients in the lowest tertile. The score had an AUC value of 0.919 to predict chronic injury, thereby outperforming baseline clinical risk factors including donor age and donor criteria, donor last serum creatinine, cold ischemia time, anastomosis time and the number of HLA mismatches (combined AUC of 0.743). Since CADI combines 6 different histopathological lesions, we additionally evaluated MRS for each lesion individually. MRS was higher in recipients with interstitial fibrosis (P<0.00001), vascular intima thickening (P=0.003) and glomerulosclerosis (P=0.0001) on the 1-year protocol-specified biopsies. In contrast, MRS did not differ in recipients with or without inflammation (P=0.82), tubular atrophy (P=0.13) or mesangial matrix increase (P=0.77).

Second, we validated our MRS in an independent cross-sectional cohort of 46 post-reperfusion brain-dead donor kidney biopsies. We deliberately selected biopsies taken at the post-reperfusion time point, which is a later time point than for the previous 2 cohorts, to ensure robustness and clinical validity of our observations. The highest versus lowest tertile of patients had a 9-fold increased risk to develop chronic injury (95% CI, 2 to 57; P=0.005). Likewise, MRS yielded a better AUC than baseline clinical risk factors combined (AUC 0.775 versus 0.694). Interestingly, MRS also correlated with reduced allograft function at 1 year after transplantation (pre-implantation cohort: Pearson correlation or r=−0.29, P=0.03; post-reperfusion cohort: r=−0.37, P=0.009), further strengthening the clinical significance of our findings. CpG islands and individual CpGs are defined by their respective positions on the chromosomes as annotated in the Genome Reference Consortium Human Hg19 Build #37 assembly.

2.5. Ranking of Methylated CpGs Based on a LASSO Model of 1000 Iterations to Predict Outcome for CAI.

The methylation risk score (MRS) as used in the presented examples was developed and calculated based on the methylated CpGs listed for the 66 validated CpG islands, as shown above and in Table 5. To determine the number of CpGs that is minimally required to calculate an MRS with a better predictive power than the current clinical parameters, we used a LASSO model consisting of 1000 iterations to calculate the MRS based on as little CpGs as possible. Those minimal models were subsequently tested in the validation cohort to allow prediction of chronic allograft injury at one year after transplantation. Of the 1634 methylated CpGs located within the 66 CpG islands (Table 5), 413 different CpGs turned out to be relevant in the LASSO model (Table 6). The number of times that each of these 413 CpG was used in one of the 1000 LASSO models was used to rank the CpGs according to their importance in predicting the risk for chronic allograft injury via MRS. Of those 413 CpGs, 29 CpGs were used in at least 10% (100 out of 1000) of the Lasso models (Table 7), and 169 CpGs were used for the MRS in 1% of the models. Finally, from these 1000-iterations minimal models we can conclude that even 4 CpGs from the most highly-ranked CpGs (Table 7) were sufficient to acquire an MRS outperforming the clinical parameters of the validation cohort to predict chronic injury at one year after transplantation.

2.6. Methods

2.6.1. Study Design and Patients

We subjected 3 different cohorts of kidney transplants to genome-wide DNA methylation profiling: a longitudinal cohort of 13×2 paired procurement (pre-ischemia) and post-reperfusion (post-ischemia) kidney transplant biopsies, with an additional biopsy 3 or 12 months after transplantation in a subgroup (n=2×5); a second pre-implantation cohort of biopsies obtained immediately prior to implantation (n=82); a third cohort of post-reperfusion biopsies (n=46; post-reperfusion cohort). We additionally collected 10 post-reperfusion biopsies, 5 from living donor kidney transplantations versus 5 from deceased donor transplantations with long cold ischemia times to validate DNA hydroxymethylation changes through LC-MS. Machine-perfused kidneys were excluded from all cohorts. All transplant recipients gave written informed consent and the study was approved by the Ethical Review Board of the University Hospitals Leuven (S53364).

2.6.2. Epigenome-Wide Methylation Profiling

Genomic DNA was extracted from all biopsies using Allprep DNA/RNA/miRNA Universal kit (Qiagen, Hilden, Germany). For genome-wide methylation analysis, DNA was bisulphite converted using EZ DNA Methylation kit (Zymo Research, Irvine, Calif., USA) and subsequently probed for DNA methylation levels using the Illumina EPIC array (for the longitudinal and pre-implantation cohort) or the 450K array²⁴ (for the post-reperfusion cohort). TET-assisted bisulphite conversion was used for hydroxymethylation analysis, as described (Thienpont et al. 2016, Nature 537:63-68). Quality control consisted of: removal of probes for which any sample did not pass a 0.01 detection P value threshold, bead cut-off of 0.05, and removal of probes on sex chromosomes. Probe annotation was performed using Minfi (Aryee et al. 2014, Bioinformatics 30:1363-1369).

2.6.3. Gene Expression Profiling

RT-PCR was performed using OpenArray technology, a real-time PCR-based solution for high-throughput gene expression analysis (Quantstudio 12K Flex Real-Time PCR system, Thermofisher Scientific, Ghent, Belgium) for 70 transcripts that corresponded to the protein-coding genes associated with the 66 CpG islands that were hypermethylated upon ischemia at FDR<0.05 in both cohorts, and for the DNA methylation modifiers TET1, TET2, TET3, DNMT1, DNMT3A, DNMT3B, DNMT3L. Five housekeeping genes (B2M, 18S, TBP, RPL13A, YWHAZ) were selected according to the literature, of which 18S, TBP and YWHAZ were considered adequate based on the gene expression changes pre- versus post-ischemia. Five of 70 transcripts failed.

2.6.4. Statistical Analyses

Statistical analyses were performed using RStudio (version 0.99). Raw methylation data were normalised using BMIQ and batch corrected using Combat, with the ChAMP pipeline (Morris et al. 2014, Bioinformatics 30:428-430). Methylation levels (beta-values) were logarithmically transformed to M-values for all statistical tests, unless stated otherwise. Results are presented as P values and FDR values using the Benjamini and Hochberg method. LC-MS to determine unmethylated C, 5mC and 5hmC concentrations in the transplant genome was performed as described (Thienpont et al. 2016, Nature 537:63-68). In the longitudinal cohort, we compared DNA methylation and hydroxymethylation levels pre- versus post-ischemia overall using Wilcoxon signed-rank and paired t-tests respectively, and subsequently at CpG-site level. In the pre-implantation cohort, we examined the effect of cold ischemia time expressed as a continuous variable (in hours) on DNA methylation for all CpGs using linear regression adjusted for donor age and gender, since age and gender are major determinants of the DNA methylome. In addition, individual CpGs were grouped according to their associated CpG island (including shores and shelves) and similar analyses were performed for CpG islands: in the longitudinal cohort by paired t-tests per island and in the pre-implantation cohort using a linear mixed model, adjusted for donor age and gender, and with transplant identifier as a random effect. To evaluate locus-specifically whether changes in 5mC are mirrored by inverse changes in 5hmC in the longitudinal cohort, 5mC levels for this particular analysis were estimated by subtracting 5hmC from 5mC, as described previously (Thienpont et al. 2016, Nature 537:63-68), since 5mC and 5hmC are both measured as 5mC after bisulphite conversion.

Hyper- versus hypomethylation events were compared using binomial tests. Overlap between cohorts was investigated by χ² analysis. We annotated ischemia-hypermethylated probes in both cohorts to their chromatin state using chromHMM data annotated for human fetal kidney (Kundaje et al. 2015, Nature 518:317-330). Pathway analysis was performed using DAVID, gene ontology enrichment using topGO in R.

Gene expression in each post-ischemia sample was calculated relative to the expression of the reference pre-ischemia sample, using the ΔΔCt method with log 2 transformation.

Ischemia-induced hypermethylation was correlated with the CADI score in protocol-specified allograft biopsies obtained at 3 months and 1 year after transplantation. Analyses were done unadjusted and adjusted for donor age (the major determinant of chronic injury) (Stegall et al. 2011, Am J Transplant 11:698-707) and donor gender (which influences DNA methylation), and in a separate analysis also for cold and warm ischemia time.

Methylation values are usually expressed as “beta values”. Beta values ((3) are the estimate of methylation level using the ratio of intensities between methylated and unmethylated alleles. β values range between 0 and 1, with β=0 being unmethylated and β=1 being fully methylated.

A methylation risk score (MRS) was developed to predict chronic injury (CADI-score>2) at 1 year after transplantation. For this, we first selected all 66 CpG islands that were hypermethylated due to transplantation-induced ischemia in two cohorts (i.e., the paired biopsy cohort and the pre-implantation biopsy cohort). These 66 CpG islands contained 1,634 CpGs. From these, we selected all 1,238 CpGs that are also measured using 450K arrays (to allow our 850K array-based methylation data to be replicated in the post-implantation biopsy cohort, which was profiled using 450K Illumina arrays only). Then, we correlated methylation (beta) values from each of the 1,238 CpGs located in these 66 CpG islands with chronic injury (CADI>2) in the pre-implantation cohort. For this, a logistic regression model containing each of the 1238 CpGs was fit using ridge regression to penalize the coefficient estimates. Ridge regression was chosen because it is better suited for logistic models with many input variables and also because it can handle input variables that are dependent from each other (which is necessary here because CpGs that belong to a CpG island are often co-regulated at the methylation level). This resulted in a logistic model, in which a coefficient was assigned to each individual CpG. Next, the methylation risk score was defined as the sum of methylation (beta) values at each CpG in 66 ischemia-hypermethylated CpG islands, weighted by marker-specific effect sizes (i.e., multiplied by the coefficient obtained for this CpG in the logistic regression model). The DNA methylation risk score was correlated to allograft function at 1 year after transplantation using the estimated glomerular filtration rate (eGFR) calculated by the MDRD formula (Poggio et al. 2006, Am J Transplant 6:100-108).

The formula for calculating the methylation risk score (MRS) as outlined above is: MRS=intercept+c₁β₁+c₂β₂+c₃β₃+ . . . +cₙβₙ. The methylation risk score, consisting of the same coefficients that were determined in the pre-implantation discovery cohort (c₁, c₂, c₃, c₄, . . . , c₁₂₃₈) was subsequently validated in the post-reperfusion cohort.

The MRS can be calculated for n methylation markers wherein n is the actual number of methylation markers. For instance, n=4, 5, 6, 7, 8, 9, 10, 11, 12, 13, 14, 15, 16, 17, 18, 19, 20, 21, 22, 23, 24, 25, 26, 27, 28, 29 or more (see description).

TABLE 1
DNA methylation changes at the genome-wide level in the kidney. Top 50 differentially methylated CpG sites (out of 92 778) with
significant linear association (FDR < 0.05) between age of kidney donor and the extent of methylation in a kidney biopsy.

| | | | | | | | Relation | |
|---|---|---|---|---|---|---|---|---|
| | CpG name | p-value | FDR | chr | pos | CpG island | to Island | UCSC RefGene Accession |
| 1 | cg03036557 | 2.38E−37 | 1.91E−31 | chr13 | 92050720 | chr13: 92051153-92051716 | N_Shore | NM_004466 |
| 2 | cg15815333 | 1.96E−36 | 7.89E−31 | chr10 | 22765209 | chr10: 22764708-22767050 | Island | |
| 3 | cg07060551 | 1.01E−35 | 2.70E−30 | chr19 | 51198381 | chr19: 51198143-51198460 | Island | NM_016148 |
| 4 | cg14692377 | 3.06E−35 | 6.15E−30 | chr17 | 28562685 | chr17: 28562387-28563186 | Island | NM_001045; NM_001045 |
| 5 | cg17251658 | 5.42E−35 | 8.72E−30 | chr16 | 49315754 | chr16: 49314037-49316543 | Island | NM_004352 |
| 6 | cg23394610 | 6.70E−35 | 8.97E−30 | chr2 | 98703695 | chr2: 98703354-98703889 | Island | NM_144992; NM_144992 |
| 7 | cg09330898 | 1.04E−34 | 1.20E−29 | chr2 | 98703682 | chr2: 98703354-98703889 | Island | NM_144992; NM_144992 |
| 8 | cg11705975 | 2.30E−34 | 2.22E−29 | chr10 | 120354248 | chr10: 120353692-120355821 | Island | NM_004248 |
| 9 | cg24724428 | 2.48E−34 | 2.22E−29 | chr6 | 11044888 | chr6: 11043913-11045206 | Island | NM_017770 |
| 10 | cg11667847 | 4.92E−34 | 3.95E−29 | chr9 | 140033911 | chr9: 140033235-140034176 | Island | NM_000832; NM_001185091; |
| | | | | | | | | NM_007327; NM_021569; |
| | | | | | | | | NM_001185090; NM_000832; |
| | | | | | | | | NM_001185091; NM_007327; |
| | | | | | | | | NM_021569; NM_001185090 |
| 11 | cg12100751 | 8.93E−34 | 6.53E−29 | chr1 | 109203672 | chr1: 109203593-109204378 | Island | NM_001102592; NM_001102592; |
| | | | | | | | | NM_144584 |
| 12 | cg07544187 | 1.66E−33 | 1.11E−28 | chr19 | 19651235 | chr19: 19650683-19651274 | Island | NM_153221 |
| 13 | cg12678562 | 2.38E−33 | 1.47E−28 | chr13 | 92050726 | chr13: 92051153-92051716 | N_Shore | NM_004466 |
| 14 | cg22454769 | 4.43E−33 | 2.54E−28 | chr2 | 106015767 | chr2: 106014878-106015884 | Island | NM_001039492; NM_001450; |
| | | | | | | | | NM_201557; NM_201555 |
| 15 | cg16867657 | 5.24E−33 | 2.81E−28 | chr6 | 11044877 | chr6: 11043913-11045206 | Island | NM_017770 |
| 16 | cg21953332 | 7.09E−33 | 3.56E−28 | chr1 | 109203680 | chr1: 109203593-109204378 | Island | NM_001102592; NM_144584; |
| | | | | | | | | NM_001102592 |
| 17 | cg15149655 | 7.80E−33 | 3.69E−28 | chr2 | 98703698 | chr2: 98703354-98703889 | Island | NM_144992; NM_144992 |
| 18 | cg07553761 | 8.54E−33 | 3.81E−28 | chr3 | 160167977 | chr3: 160167184-160168200 | Island | NM_173084 |
| 19 | cg07806886 | 1.96E−32 | 8.30E−28 | chr3 | 120626899 | chr3: 120626880-120627579 | Island | NM_014980 |
| 20 | cg23606718 | 3.16E−32 | 1.27E−27 | chr2 | 131513927 | chr2: 131513363-131514183 | Island | NM_152698; NM_001105194; |
| | | | | | | | | NM_001105195; NM_001105194; |
| | | | | | | | | NM_001105193; NM_001105195 |
| 21 | cg06210197 | 6.11E−32 | 2.34E−27 | chr8 | 24771256 | chr8: 24770908-24772547 | Island | NM_001105541; NM_005382 |
| 22 | cg16822939 | 7.98E−32 | 2.91E−27 | chr20 | 43922174 | chr20: 43921949-43922642 | Island | NM_030592; NM_003833; |
| | | | | | | | | NM_030590 |
| 23 | cg14693555 | 1.09E−31 | 3.80E−27 | chr16 | 49315752 | chr16: 49314037-49316543 | Island | NM_004352 |
| 24 | cg21620282 | 1.36E−31 | 4.55E−27 | chr14 | 93389628 | chr14: 93389245-93389899 | Island | NM_001301690; NM_001275; |
| | | | | | | | | NM_001301690; NM_001275 |
| 25 | cg10906284 | 3.04E−31 | 9.77E−27 | chr12 | 63544430 | chr12: 63543636-63544967 | Island | NM_000706 |
| 26 | cg23091758 | 3.88E−31 | 1.20E−26 | chr11 | 9025767 | chr11: 9025095-9026315 | Island | NM_020645 |
| 27 | cg01867395 | 4.87E−31 | 1.41E−26 | chr11 | 31839628 | chr11: 31839363-31839813 | Island | NM_001127612 |
| 28 | cg03389653 | 4.96E−31 | 1.41E−26 | chr16 | 49316197 | chr16: 49314037-49316543 | Island | NM_004352 |
| 29 | cg12546181 | 5.08E−31 | 1.41E−26 | chr19 | 48902029 | chr19: 48901804-48902123 | Island | NM_000836 |
| 30 | cg12841266 | 6.93E−31 | 1.86E−26 | chr3 | 9594093 | chr3: 9594068-9594328 | Island | NM_198560 |
| 31 | cg25090514 | 7.58E−31 | 1.97E−26 | chr5 | 2038743 | chr5: 2038527-2038949 | Island | |
| 32 | cg26885220 | 1.82E−30 | 4.56E−26 | chr1 | 65775570 | chr1: 65775018-65775746 | Island | NM_014787 |
| 33 | cg05376147 | 2.14E−30 | 5.22E−26 | chr9 | 139097007 | chr9: 139096665-139096993 | S_Shore | NM_178138 |
| 34 | cg15123984 | 2.75E−30 | 6.50E−26 | chr5 | 174151634 | chr5: 174151478-174152364 | Island | NM_002449; NM_002449 |
| 35 | cg11018337 | 3.34E−30 | 7.68E−26 | chr10 | 8095495 | chr10: 8091374-8098329 | Island | NR_024256; NM_002051; |
| | | | | | | | | NM_001002295; NR_024255 |
| 36 | cg14585700 | 3.55E−30 | 7.92E−26 | chr9 | 37027605 | chr9: 37026222-37028014 | Island | NM_016734 |
| 37 | cg13570972 | 4.31E−30 | 9.36E−26 | chr11 | 31839632 | chr11: 31839363-31839813 | Island | NM_001127612 |
| 38 | cg15593298 | 4.49E−30 | 9.49E−26 | chr3 | 142681996 | chr3: 142681137-142683268 | Island | NM_198504 |
| 39 | cg10091994 | 5.40E−30 | 1.11E−25 | chr12 | 4381803 | chr12: 4378366-4382222 | Island | NM_001759 |
| 40 | cg11229185 | 6.04E−30 | 1.21E−25 | chr10 | 22625274 | chr10: 22623350-22625875 | Island | |
| 41 | cg02623400 | 8.48E−30 | 1.66E−25 | chr1 | 50513749 | chr1: 50513644-50514320 | Island | NM_001144777; NM_001144777 |
| 42 | cg10548038 | 9.18E−30 | 1.76E−25 | chr13 | 92050731 | chr13: 92051153-92051716 | N_Shore | NM_004466 |
| 43 | cg20899581 | 1.18E−29 | 2.20E−25 | chr6 | 27841230 | chr6: 27841001-27841244 | Island | NM_003546; NM_003533 |
| 44 | cg00048759 | 1.36E−29 | 2.48E−25 | chr7 | 99775422 | chr7: 99774733-99775583 | Island | NM_012447; NM_152742 |
| 45 | cg18740893 | 1.80E−29 | 3.21E−25 | chr3 | 136538934 | chr3: 136537559-136539204 | Island | NM_001097600; NM_025246; |
| | | | | | | | | NM_001097599 |
| 46 | cg16703882 | 1.85E−29 | 3.23E−25 | chr3 | 157823479 | chr3: 157822973-157823836 | Island | NM_006884; NM_003030; |
| | | | | | | | | NM_001163678 |
| 47 | cg05236677 | 2.78E−29 | 4.75E−25 | chr8 | 99952217 | chr8: 99952020-99954686 | Island | |
| 48 | cg15504992 | 2.87E−29 | 4.81E−25 | chr8 | 67874970 | chr8: 67873388-67875600 | Island | NM_001193502 |
| 49 | cg06022942 | 3.91E−29 | 6.41E−25 | chr10 | 8095484 | chr10: 8091374-8098329 | Island | NR_024256; NM_002051; |
| | | | | | | | | NM_001002295; NR_024255 |
| 50 | cg22353329 | 4.94E−29 | 7.93E−25 | chr17 | 77814357 | chr17: 77812991-77819081 | Island | NM_003655 |

TABLE 2
DNA methylation changes at the genome-wide level in the kidney. Top 99 differentially methylated regions
(DMRs) surpassing a \check{S}idák multiple testing corrected P-value of 0.0001. A DMR was
defined as nearby located CpGs demonstrating the same age (of kidney donor)-associated methylation changes.

| | | | | | | no of | |
|---|---|---|---|---|---|---|---|
| | | | | sidak p | min p | CpG | |
| | chromosome | start | end | value | value | probes | all genes names |
| 1 | chr8 | 24768802 | 24774727 | 9.83E−59 | 1.54E−80 | 32 | RP11-624C23.1, GS1-72M22.1, NEFM |
| 2 | chr10 | 22621287 | 22627667 | 5.41E−50 | 1.87E−78 | 26 | RP11-573G6.9, RP11-573G6.8 |
| 3 | chr8 | 145101998 | 145107857 | 9.55E−64 | 4.37E−76 | 21 | CTD-3065J16.6, OPLAH |
| 4 | chr3 | 147121892 | 147132559 | 9.74E−60 | 4.85E−74 | 71 | ZIC4, ZIC1 |
| 5 | chr14 | 29234282 | 29238731 | 1.83E−68 | 2.41E−73 | 30 | RP11-966I7.1, FOXG1 |
| 6 | chr16 | 67194079 | 67203222 | 1.21E−49 | 2.78E−68 | 39 | TRADD, FBXL8, HSF4, RP11-5A19.5 |
| 7 | chr8 | 70980488 | 70984917 | 1.66E−51 | 1.39E−63 | 22 | PRDM14 |
| 8 | chr11 | 31817810 | 31849262 | 2.54E−82 | 1.45E−62 | 155 | PAX6, RCN1 |
| 9 | chr16 | 2285252 | 2289673 | 3.73E−44 | 1.67E−57 | 25 | RP11-304L19.12, E4F1, DNASE1L2, ECI1 |
| 10 | chr15 | 41950389 | 41954252 | 2.21E−37 | 3.23E−56 | 17 | MGA |
| 11 | chr8 | 145697495 | 145702343 | 4.12E−22 | 1.85E−55 | 20 | KIFC2, FOXH1 |
| 12 | chr2 | 98701516 | 98704858 | 4.73E−49 | 1.93E−53 | 18 | VWA3B |
| 13 | chr1 | 91300215 | 91303544 | 3.89E−49 | 3.65E−53 | 18 | RP4-665J23.1 |
| 14 | chr16 | 66612290 | 66615649 | 2.19E−38 | 5.98E−53 | 17 | RP11-403P17.2, CKLF-CMTM1, CMTM1, CMTM2 |
| 15 | chr7 | 155595288 | 155599625 | 1.08E−31 | 7.00E−52 | 12 | SHH |
| 16 | chr19 | 36244945 | 36250163 | 1.04E−19 | 7.09E−52 | 30 | AC002398.12, AC002398.9, LIN37, HSPB6, C19orf55 |
| 17 | chr13 | 28363281 | 28372843 | 7.93E−30 | 2.74E−51 | 33 | GSX1 |
| 18 | chr1 | 151809534 | 151814071 | 2.89E−29 | 4.22E−49 | 19 | C2CD4D |
| 19 | chr2 | 74725040 | 74728623 | 2.23E−36 | 2.20E−48 | 18 | AC005041.17, LBX2 |
| 20 | chr15 | 68110793 | 68129069 | 3.29E−47 | 3.46E−48 | 76 | RP11-34F13.3, RP11-34F13.2, SKOR1 |
| 21 | chr16 | 66636477 | 66639593 | 6.46E−45 | 6.34E−48 | 25 | CMTM3 |
| 22 | chr3 | 120626107 | 120628544 | 2.57E−42 | 1.11E−47 | 13 | STXBP5L |
| 23 | chr1 | 63782395 | 63796486 | 1.52E−44 | 3.42E−47 | 65 | LINC00466, RP4-792G4.2, FOXD3 |
| 24 | chr2 | 162270453 | 162285454 | 6.87E−42 | 3.94E−47 | 63 | AC009487.4, AC009487.5, TBR1, SLC4A10 |
| 25 | chr2 | 241457438 | 241460664 | 1.87E−40 | 1.24E−46 | 9 | ANKMY1 |
| 26 | chr19 | 46915570 | 46918365 | 2.21E−23 | 1.69E−46 | 16 | CCDC8 |
| 27 | chr17 | 62773012 | 62778413 | 3.61E−17 | 6.55E−46 | 28 | hsa-mir-6080, RP11-927P21.4, PLEKHM1P |
| 28 | chr6 | 27774865 | 27778836 | 8.45E−43 | 8.75E−46 | 25 | HIST1H4PS1, HIST1H2BL, HIST1H2AI, HIST1H3H |
| 29 | chr5 | 134361614 | 134372398 | 1.45E−39 | 1.30E−45 | 42 | PITX1, C5orf66 |
| 30 | chr2 | 27528349 | 27532725 | 1.51E−33 | 1.34E−45 | 23 | TRIM54, UCN, MPV17 |
| 31 | chr15 | 83951663 | 83956766 | 2.29E−11 | 2.09E−45 | 37 | RP11-382A20.4, BNC1 |
| 32 | chr19 | 18979397 | 18981378 | 1.00E−45 | 4.56E−45 | 6 | CERS1, GDF1 |
| 33 | chr20 | 42873864 | 42876939 | 2.61E−41 | 1.33E−44 | 17 | GDAP1L1 |
| 34 | chr2 | 63273436 | 63287686 | 1.10E−35 | 2.93E−44 | 93 | AC009501.4, EHBP1, OTX1 |
| 35 | chr1 | 6268709 | 6270967 | 1.10E−20 | 5.52E−44 | 12 | RPL22, RNF207 |
| 36 | chr3 | 9744908 | 9747183 | 9.81E−36 | 1.27E−43 | 13 | CPNE9 |
| 37 | chr4 | 158140839 | 158144318 | 7.59E−33 | 1.55E−43 | 24 | GRIA2 |
| 38 | chr2 | 124782117 | 124783698 | 1.06E−43 | 5.15E−43 | 12 | AC079154.1, CNTNAP5 |
| 39 | chr17 | 6898315 | 6900356 | 3.69E−32 | 7.37E−43 | 22 | AC027763.2, ALOX12, RP11-589P10.7, RP11-589P10.5 |
| 40 | chr13 | 79168044 | 79171679 | 2.38E−35 | 8.19E−43 | 23 | RNF219-AS1, RP11-52L5.6 |
| 41 | chr2 | 119599067 | 119613877 | 1.40E−27 | 8.29E−43 | 70 | EN1 |
| 42 | chr15 | 60284643 | 60298900 | 2.96E−39 | 3.40E−42 | 56 | FOXB1 |
| 43 | chr19 | 17436863 | 17440072 | 1.71E−31 | 5.96E−42 | 9 | ANo8 |
| 44 | chr15 | 41803428 | 41806588 | 4.56E−32 | 8.20E−42 | 20 | LTK |
| 45 | chr7 | 155246474 | 155252796 | 1.82E−31 | 2.16E−41 | 26 | AC008060.8, EN2 |
| 46 | chr1 | 91180913 | 91187268 | 1.14E−34 | 2.41E−41 | 26 | BARHL2 |
| 47 | chr2 | 172943770 | 172953925 | 2.01E−42 | 4.04E−41 | 50 | METAP1D, DLX1 |
| 48 | chr4 | 111530900 | 111545628 | 1.63E−33 | 5.66E−41 | 55 | RP11-380D23.2, PITX2 |
| 49 | chr2 | 25472665 | 25476664 | 6.21E−30 | 6.07E−41 | 15 | DNMT3A |
| 50 | chr11 | 847547 | 850952 | 4.26E−34 | 7.24E−41 | 9 | TSPAN4 |
| 51 | chr2 | 175195899 | 175210231 | 3.11E−34 | 7.37E−41 | 51 | SP9, AC018470.1 |
| 52 | chr10 | 118889589 | 118901190 | 3.38E−34 | 9.49E−41 | 48 | VAX1 |
| 53 | chr14 | 24639764 | 24642358 | 1.61E−36 | 1.33E−40 | 19 | REC8 |
| 54 | chr13 | 100546327 | 100549017 | 7.78E−29 | 1.67E−40 | 13 | CLYBL |
| 55 | chr4 | 4852986 | 4874042 | 1.09E−34 | 2.21E−40 | 105 | MSX1 |
| 56 | chr20 | 50719777 | 50722929 | 3.66E−39 | 2.67E−40 | 17 | ZFP64 |
| 57 | chr4 | 9781782 | 9783965 | 9.25E−33 | 5.00E−40 | 14 | SLC2A9, DRD5 |
| 58 | chr1 | 1564788 | 1567820 | 1.27E−28 | 6.30E−40 | 16 | MIB2, MMP23B |
| 59 | chr10 | 8084742 | 8102583 | 1.41E−29 | 1.28E−39 | 84 | GATA3, GATA3-AS1, RP11-379F12.4, RP11-379F12.3 |
| 60 | chr6 | 105387483 | 105389370 | 5.39E−33 | 2.50E−39 | 9 | LINC00577 |
| 61 | chr4 | 147557774 | 147561900 | 2.26E−28 | 3.31E−39 | 20 | POU4F2, AC093887.1 |
| 62 | chr10 | 28029852 | 28037266 | 5.17E−23 | 4.02E−39 | 43 | MKX, RP11-360I20.2 |
| 63 | chr6 | 27098478 | 27103185 | 1.59E−33 | 5.90E−39 | 21 | HIST1H2BJ, HIST1H2AG |
| 64 | chr2 | 223154176 | 223173061 | 1.38E−36 | 5.90E−39 | 79 | PAX3, CCDC140 |
| 65 | chr1 | 25252163 | 25259034 | 2.17E−24 | 7.66E−39 | 41 | RUNX3 |
| 66 | chr3 | 9592686 | 9595646 | 3.13E−16 | 9.68E−39 | 11 | LHFPL4 |
| 67 | chr3 | 138654370 | 138669434 | 3.26E−34 | 1.22E−38 | 61 | FOXL2, C3orf72, RP11-548O1.3 |
| 68 | chr10 | 22632995 | 22635143 | 3.93E−33 | 1.40E−38 | 17 | SPAG6 |
| 69 | chr13 | 79174811 | 79179373 | 1.61E−26 | 2.95E−38 | 21 | RNF219-AS1, POU4F1 |
| 70 | chr4 | 174436918 | 174446201 | 2.05E−32 | 6.37E−38 | 31 | HAND2 |
| 71 | chr6 | 10881086 | 10888129 | 7.86E−21 | 1.12E−37 | 42 | RP11-637O19.2, SYCP2L, RP11-637O19.3, GCM2 |
| 72 | chr16 | 69139327 | 69141478 | 2.05E−25 | 1.26E−37 | 21 | HAS3 |
| 73 | chr16 | 56664646 | 56672722 | 4.55E−26 | 1.46E−37 | 32 | MT1JP, AC026461.1, MT1M, MT1A |
| 74 | chr6 | 30094300 | 30095802 | 1.50E−37 | 3.77E−37 | 25 | NA |
| 75 | chr19 | 13120555 | 13125988 | 1.01E−20 | 3.79E−37 | 21 | CTC-239J10.1, NFIX |
| 76 | chr6 | 26224013 | 26226256 | 3.05E−34 | 4.35E−37 | 20 | HIST1H3E |
| 77 | chr3 | 160166748 | 160168922 | 3.04E−31 | 7.60E−37 | 18 | RP11-432B6.3, TRIM59 |
| 78 | chr12 | 54068942 | 54072736 | 5.74E−28 | 1.07E−36 | 29 | ATP5G2 |
| 79 | chr15 | 65686654 | 65690551 | 2.47E−28 | 1.18E−36 | 11 | IGDCC4 |
| 80 | chr7 | 20822829 | 20827982 | 3.25E−25 | 1.31E−36 | 21 | SP8 |
| 81 | chr11 | 636167 | 641042 | 8.93E−29 | 1.33E−36 | 19 | DRD4 |
| 82 | chr2 | 182542510 | 182550065 | 4.31E−21 | 1.43E−36 | 38 | AC013733.3, CERKL, NEUROD1 |
| 83 | chr3 | 147102840 | 147116807 | 1.26E−30 | 1.77E−36 | 53 | ZIC4-AS1, ZIC4, ZIC1 |
| 84 | chr10 | 77155143 | 77159055 | 6.88E−23 | 1.86E−36 | 16 | RP11-399K21.11, ZNF503 |
| 85 | chr1 | 91188999 | 91192803 | 7.23E−31 | 2.99E−36 | 28 | NA |
| 86 | chr10 | 128993051 | 128995478 | 1.11E−32 | 3.50E−36 | 16 | DOCK1, FAM196A |
| 87 | chr12 | 99287129 | 99290378 | 6.22E−19 | 3.72E−36 | 18 | ANKS1B |
| 88 | chr1 | 154473340 | 154476659 | 7.90E−27 | 4.11E−36 | 13 | SHE, TDRD10 |
| 89 | chr14 | 60972853 | 60978852 | 3.72E−27 | 5.16E−36 | 32 | C14orf39, SIX6 |
| 90 | chr13 | 49791335 | 49796489 | 2.01E−21 | 6.32E−36 | 14 | MLNR |
| 91 | chr19 | 13207239 | 13215387 | 1.31E−26 | 8.27E−36 | 29 | NFIX, LYL1 |
| 92 | chr9 | 79627216 | 79635912 | 3.54E−39 | 9.38E−36 | 26 | FOXB2 |
| 93 | chr11 | 82443149 | 82446219 | 6.03E−19 | 9.70E−36 | 15 | FAM181B |
| 94 | chr7 | 35291644 | 35301861 | 1.61E−32 | 1.52E−35 | 36 | AC009531.2, TBX20 |
| 95 | chr1 | 50880864 | 50893984 | 9.31E−39 | 1.52E−35 | 60 | DMRTA2 |
| 96 | chr17 | 37760173 | 37767494 | 5.36E−24 | 1.88E−35 | 32 | NEUROD2 |
| 97 | chr6 | 108484512 | 108492769 | 1.11E−37 | 3.15E−35 | 45 | OSTM1, NR2E1 |
| 98 | chr9 | 37024153 | 37027815 | 3.63E−34 | 3.72E−35 | 9 | PAX5 |
| 99 | chr16 | 47175842 | 47178957 | 1.44E−31 | 4.29E−35 | 15 | RP11-329J18.2, NETO2 |

TABLE 3
DNA methylation changes at the genome-wide level in the kidney. Top 50 differentially methylated CpG sites of the 31 805
(out of 92 778) CpG sites correlated (at FDR < 0.05) with glomerulosclerosis at one year after kidney transplantation.

| | | | | | | | Relation | |
|---|---|---|---|---|---|---|---|---|
| | | | | | | | to CpG | |
| | CpG name | p value | FDR | chr | pos | CpG island | island | UCSC RefGene Accession |
| 1 | cg06720949 | 6.29E−09 | 0.000198733 | chr19 | 45381937 | | OpenSea | NM_001042724; NM_002856 |
| 2 | cg17271223 | 6.43E−09 | 0.000198733 | chr7 | 132957775 | | OpenSea | NM_021807; NM_001037126 |
| 3 | cg19044229 | 2.81E−09 | 0.000198733 | chr11 | 65374955 | chr11: 65374699-65375308 | Island | NM_002419 |
| 4 | cg00061520 | 2.17E−08 | 0.000251175 | chr16 | 4070371 | | OpenSea | NM_001116 |
| 5 | cg01900755 | 1.59E−08 | 0.000251175 | chr6 | 64350180 | chr6: 64345490-64346465 | S_Shelf | NM_001290259; NM_001290260 |
| 6 | cg11782729 | 1.38E−08 | 0.000251175 | chr17 | 576564 | | OpenSea | NM_018289; NM_001128159 |
| 7 | cg16883450 | 2.05E−08 | 0.000251175 | chr1 | 245911763 | | OpenSea | |
| 8 | cg26096304 | 2.08E−08 | 0.000251175 | chr5 | 179950726 | | OpenSea | NM_015455 |
| 9 | cg02665578 | 2.48E−08 | 0.000255242 | chr20 | 62153239 | chr20: 62153066-62153270 | Island | NM_024299 |
| 10 | cg02422197 | 3.64E−08 | 0.000307341 | chr14 | 24771598 | chr14: 24768620-24769364 | S_Shelf | NM_001286367; NM_174913 |
| 11 | cg23083046 | 3.50E−08 | 0.000307341 | chr12 | 19903394 | | OpenSea | |
| 12 | cg05726208 | 5.09E−08 | 0.000393751 | chr11 | 129817871 | | OpenSea | NM_199439; NM_199438; |
| | | | | | | | | NM_020228; NM_199437 |
| 13 | cg19610659 | 6.09E−08 | 0.000434432 | chr12 | 53699282 | | OpenSea | NM_021640 |
| 14 | cg08606493 | 9.06E−08 | 0.000443463 | chr8 | 27308082 | | OpenSea | NM_173176; NM_173175; |
| | | | | | | | | NM_173174; NM_004103 |
| 15 | cg09195780 | 9.50E−08 | 0.000443463 | chr7 | 7811979 | | OpenSea | NM_001302350; NM_001302348; |
| | | | | | | | | NM_001302349 |
| 16 | cg10288719 | 8.24E−08 | 0.000443463 | chr2 | 128622541 | | OpenSea | NM_001199140; NM_031445 |
| 17 | cg11903872 | 1.00E−07 | 0.000443463 | chr9 | 138844422 | | OpenSea | NM_016172 |
| 18 | cg12471836 | 9.23E−08 | 0.000443463 | chr16 | 2480552 | chr16: 2478686-2479968 | S_Shore | NM_001761 |
| 19 | cg20626616 | 8.61E−08 | 0.000443463 | chr3 | 10332506 | | OpenSea | NM_016362; NR_024137; |
| | | | | | | | | NR_024134; NM_001134941; |
| | | | | | | | | NR_024136; NR_024133; |
| | | | | | | | | NR_024132; NR_024146; |
| | | | | | | | | NR_024135; NR_024145; |
| | | | | | | | | NR_004431; NM_001134945; |
| | | | | | | | | NM_001134946; NR_024138; |
| | | | | | | | | NM_001134944; NR_024144 |
| 20 | cg25273619 | 9.95E−08 | 0.000443463 | chr13 | 114123037 | | OpenSea | NM_001014283 |
| 21 | cg26407571 | 8.33E−08 | 0.000443463 | chr12 | 54473534 | chr12: 54473305-54473562 | Island | NR_026655; NR_026658 |
| 22 | cg09202851 | 1.20E−07 | 0.000481572 | chr11 | 567966 | chr11: 567938-569461 | Island | |
| 23 | cg14097773 | 1.20E−07 | 0.000481572 | chr13 | 114123258 | | OpenSea | NM_001014283 |
| 24 | cg14497910 | 1.29E−07 | 0.000481572 | chr13 | 114123184 | | OpenSea | NM_001014283 |
| 25 | cg22960616 | 1.30E−07 | 0.000481572 | chr11 | 71188716 | | OpenSea | NM_018161 |
| 26 | cg01649611 | 1.50E−07 | 0.000485169 | chr2 | 43521066 | | OpenSea | NM_022065; NM_001083953 |
| 27 | cg09589331 | 1.36E−07 | 0.000485169 | chr3 | 71256044 | | OpenSea | NM_001244814; NM_001244812; |
| | | | | | | | | NM_001244816; NM_001244808; |
| | | | | | | | | NM_032682; NM_001244810; |
| | | | | | | | | NM_001012505 |
| 28 | cg10255171 | 1.64E−07 | 0.000485169 | chr6 | 16328344 | chr6: 16328169-16328563 | Island | NM_001128164; NM_000332 |
| 29 | cg14982576 | 1.68E−07 | 0.000485169 | chr13 | 114123001 | | OpenSea | NM_001014283 |
| 30 | cg21541534 | 1.72E−07 | 0.000485169 | chr4 | 86684656 | | OpenSea | NM_001025616 |
| 31 | cg23931819 | 1.67E−07 | 0.000485169 | chr1 | 1245076 | chr1: 1242400-1245185 | Island | NM_153339 |
| 32 | cg24332389 | 1.72E−07 | 0.000485169 | chr1 | 6558085 | chr1: 6557561-6557872 | S_Shore | NM_198681; NM_001042663 |
| 33 | cg24508633 | 1.73E−07 | 0.000485169 | chr15 | 89630675 | chr15: 89631546-89632209 | N_Shore | NM_152924; NM_007011 |
| 34 | cg03929366 | 1.84E−07 | 0.000485324 | chr8 | 105377722 | chr8: 105379566-105379986 | N_Shore | |
| 35 | cg11381106 | 1.79E−07 | 0.000485324 | chr3 | 185643153 | | OpenSea | NM_001243879; NM_004593 |
| 36 | cg15662465 | 1.88E−07 | 0.000485324 | chr13 | 70682004 | chr13: 70681732-70682219 | Island | NM_020866; NM_001286725; |
| | | | | | | | | NM_020866; NM_001286725; |
| | | | | | | | | NR_002717 |
| 37 | cg00910503 | 2.15E−07 | 0.000512442 | chr17 | 80393666 | chr17: 80393470-80393752 | Island | NM_173620 |
| 38 | cg07949722 | 2.15E−07 | 0.000512442 | chr17 | 32576670 | | OpenSea | |
| 39 | cg18982976 | 2.13E−07 | 0.000512442 | chr17 | 61116857 | | OpenSea | NM_025185; NR_036146 |
| 40 | cg03544320 | 2.30E−07 | 0.000516604 | chr4 | 5894691 | chr4: 5894071-5895116 | Island | NM_001014809 |
| 41 | cg04860664 | 2.40E−07 | 0.000516604 | chr1 | 43136315 | | OpenSea | NM_006347 |
| 42 | cg08118957 | 2.34E−07 | 0.000516604 | chr8 | 141300036 | | OpenSea | NM_001160372; NM_031466 |
| 43 | cg13573626 | 2.49E−07 | 0.000516604 | chr14 | 105858487 | | OpenSea | NM_015197; NM_001100913 |
| 44 | cg15567016 | 2.29E−07 | 0.000516604 | chr4 | 74174284 | | OpenSea | |
| 45 | cg16695176 | 2.51E−07 | 0.000516604 | chr5 | 179707569 | | OpenSea | NM_001308244; NM_001308244; |
| | | | | | | | | NM_002752; NM_139070; |
| | | | | | | | | NM_001135044; NM_139069; |
| | | | | | | | | NM_139068 |
| 46 | cg00320453 | 2.80E−07 | 0.000521901 | chr11 | 123756564 | | OpenSea | NM_001013743 |
| 47 | cg00767269 | 2.71E−07 | 0.000521901 | chr19 | 46056709 | chr19: 46056783-46057149 | N_Shore | NM_001017989; NM_025136 |
| 48 | cg02404377 | 3.02E−07 | 0.000521901 | chr11 | 20043971 | | OpenSea | NM_001111019; NM_182964; |
| | | | | | | | | NM_001111018; NM_145117 |
| 49 | cg02589501 | 2.99E−07 | 0.000521901 | chr4 | 53523850 | chr4: 53524958-53526227 | N_Shore | NM_001134223; NM_022832 |
| 50 | cg04644353 | 3.08E−07 | 0.000521901 | chr11 | 119394493 | | OpenSea | |

TABLE 4
DNA methylation changes at the genome-wide level in the kidney. Top 50 differentially methylated CpG sites of the 880 (out
of 92 778) CpG sites correlated (at FDR < 0.05) with interstitial fibrosis at one year after kidney transplantation.

| | | | | | | | Relation | |
|---|---|---|---|---|---|---|---|---|
| | | | | | | | to CpG | |
| | CpG name | p value | FDR | chr | pos | CpG island | island | UCSC_RefGene_Accession |
| 1 | cg18714712 | 6.57E−08 | 0.006095311 | chr19 | 49866917 | chr19: 49866752-49867209 | Island | NM_014419; NM_003598 |
| 2 | cg23872081 | 5.14E−07 | 0.023844286 | chr8 | 22436093 | chr8: 22436295-22437076 | N_Shore | NM_021630 |
| 3 | cg00449941 | 3.26E−05 | 0.039544818 | chr17 | 26926011 | chr17: 26925742-26926512 | Island | NM_006461; NM_006461 |
| 4 | cg00505001 | 3.26E−05 | 0.039544818 | chr13 | 36049807 | chr13: 36049570-36050159 | Island | NM_005584; NR_031646; |
| | | | | | | | | NM_015678 |
| 5 | cg00765922 | 3.45E−05 | 0.039544818 | chr1 | 156626839 | chr1: 156627342-156627576 | N_Shore | NM_021948 |
| 6 | cg01102477 | 2.26E−05 | 0.039544818 | chr2 | 97524719 | chr2: 97523356-97524186 | S_Shore | NM_016466 |
| 7 | cg01608635 | 3.84E−05 | 0.039544818 | chr15 | 89028369 | | OpenSea | |
| 8 | cg01724566 | 1.54E−05 | 0.039544818 | chr17 | 26926132 | chr17: 26925742-26926512 | Island | NM_006461 |
| 9 | cg01863682 | 3.13E−05 | 0.039544818 | chr2 | 182545771 | chr2: 182547873-182549177 | N_Shelf | NM_002500 |
| 10 | cg01885291 | 3.26E−05 | 0.039544818 | chr6 | 28984832 | chr6: 28984418-28984686 | S_Shore | |
| 11 | cg01912015 | 3.73E−05 | 0.039544818 | chr11 | 46431746 | | OpenSea | NM_001300731; NM_001267783; |
| | | | | | | | | NM_001267782; NM_017749 |
| 12 | cg02077276 | 9.41E−06 | 0.039544818 | chr11 | 14993977 | chr11: 14995128-14995908 | N_Shore | NM_001033952; NM_001033953; |
| | | | | | | | | NM_001741 |
| 13 | cg02445909 | 2.40E−05 | 0.039544818 | chr3 | 42190607 | | OpenSea | NM_001265609; NM_001265610; |
| | | | | | | | | NM_001265608; NM_001042646 |
| 14 | cg02648847 | 1.84E−05 | 0.039544818 | chr1 | 167408735 | chr1: 167408512-167409137 | Island | NM_198053; NM_000734 |
| 15 | cg02885694 | 3.47E−05 | 0.039544818 | chr7 | 100807168 | chr7: 100806279-100809064 | Island | NM_003378 |
| 16 | cg03656020 | 3.47E−05 | 0.039544818 | chr7 | 100805972 | chr7: 100806279-100809064 | N_Shore | NM_003378 |
| 17 | cg04279973 | 2.77E−05 | 0.039544818 | chr16 | 23846968 | chr16: 23846941-23848102 | Island | NM_002738; NM_212535 |
| 18 | cg04603730 | 1.14E−05 | 0.039544818 | chr13 | 96204870 | chr13: 96204691-96205496 | Island | NM_006984; NM_182848; |
| | | | | | | | | NM_001160100 |
| 19 | cg04751133 | 9.22E−06 | 0.039544818 | chr5 | 170846273 | chr5: 170845760-170848124 | Island | NM_003862 |
| 20 | cg04801617 | 1.72E−06 | 0.039544818 | chr12 | 106976843 | chr12: 106977388-06977713 | N_Shore | NM_213594 |
| 21 | cg04948892 | 9.66E−06 | 0.039544818 | chr3 | 181428462 | chr3: 181430141-181431076 | N_Shore | NR_004053; NM_003106 |
| 22 | cg04962528 | 2.15E−05 | 0.039544818 | chr14 | 21098715 | chr14: 21100838-21101043 | N_Shelf | |
| 23 | cg05214390 | 3.93E−05 | 0.039544818 | chr11 | 46354574 | chr11: 46354091-46355190 | Island | NM_201532 |
| 24 | cg05951603 | 3.30E−05 | 0.039544818 | chr12 | 57630871 | chr12: 57630106-57630469 | S_Shore | NM_020142 |
| 25 | cg06329022 | 2.95E−05 | 0.039544818 | chr17 | 26926511 | chr17: 26925742-26926512 | Island | NM_006461 |
| 26 | cg06774283 | 2.53E−05 | 0.039544818 | chr17 | 26926076 | chr17: 26925742-26926512 | Island | NM_006461 |
| 27 | cg07063068 | 2.38E−05 | 0.039544818 | chr14 | 91711033 | | OpenSea | NM_003485 |
| 28 | cg07065803 | 3.10E−06 | 0.039544818 | chr11 | 45921557 | chr11: 45921387-45922167 | Island | NM_005456 |
| 29 | cg07096772 | 1.43E−05 | 0.039544818 | chr2 | 240884729 | | OpenSea | |
| 30 | cg07274618 | 1.47E−05 | 0.039544818 | chr17 | 74070698 | chr17: 74070404-74073530 | Island | NM_003857 |
| 31 | cg07298257 | 3.96E−05 | 0.039544818 | chr16 | 28583885 | | OpenSea | NM_138414 |
| 32 | cg07563569 | 8.41E−06 | 0.039544818 | chr17 | 47653309 | chr17: 47653211-47654369 | Island | NM_007225; NM_007225 |
| 33 | cg07647164 | 1.11E−05 | 0.039544818 | chr1 | 214360690 | chr1: 214360607-214360965 | Island | |
| 34 | cg08332990 | 4.01E−05 | 0.039544818 | chr4 | 997351 | chr4: 995482-997541 | Island | NM_000203 |
| 35 | cg08696866 | 3.96E−06 | 0.039544818 | chr2 | 176961907 | chr2: 176962179-176962487 | N_Shore | |
| 36 | cg08812189 | 1.19E−05 | 0.039544818 | chr3 | 147110367 | chr3: 147108511-147111703 | Island | NM_001168378; NR_033118; |
| | | | | | | | | NR_033119; NM_032153; |
| | | | | | | | | NM_001168379 |
| 37 | cg09620840 | 2.90E−05 | 0.039544818 | chr6 | 30458149 | chr6: 30457369-30458175 | Island | NM_005516 |
| 38 | cg10239194 | 7.22E−06 | 0.039544818 | chr16 | 3233298 | chr16: 3232835-3234048 | Island | |
| 39 | cg10305311 | 3.53E−05 | 0.039544818 | chr13 | 96204873 | chr13: 96204691-96205496 | Island | NM_006984; NM_182848; |
| | | | | | | | | NM_001160100 |
| 40 | cg10500512 | 2.83E−05 | 0.039544818 | chr20 | 22564041 | chr20: 22562736-22566104 | Island | NM_021784; NM_153675 |
| 41 | cg10927449 | 3.45E−05 | 0.039544818 | chr2 | 63286621 | chr2: 63285949-63287097 | Island | |
| 42 | cg10992014 | 2.76E−05 | 0.039544818 | chr6 | 121758817 | | OpenSea | NM_000165 |
| 43 | cg11178170 | 4.91E−06 | 0.039544818 | chr4 | 184427252 | chr4: 184425262-184427628 | Island | NM_001564 |
| 44 | cg11471138 | 1.52E−05 | 0.039544818 | chr5 | 179918766 | chr5: 179921201-179922179 | N_Shelf | |
| 45 | cg12064947 | 3.40E−06 | 0.039544818 | chr15 | 41220983 | chr15: 41217789-41223180 | Island | NM_019074 |
| 46 | cg12402251 | 6.93E−06 | 0.039544818 | chr8 | 91094811 | | OpenSea | NM_004929 |
| 47 | cg12534549 | 1.99E−05 | 0.039544818 | chr19 | 41208535 | | OpenSea | NM_001142555; NM_024876 |
| 48 | cg13156931 | 1.92E−05 | 0.039544818 | chr13 | 28554471 | chr13: 28554427-28555065 | Island | NM_001105577 |
| 49 | cg13273128 | 1.41E−06 | 0.039544818 | chr2 | 45241620 | chr2: 45240372-45241579 | S_Shore | |
| 50 | cg13349607 | 1.01E−05 | 0.039544818 | chr7 | 120962655 | | OpenSea | |

TABLE 5
Validated CpG islands (66) containing multiple hypermethylated CpGs (ischemia-induced).

| | | longitudinal cohort | | | pre-implantation cohort | | |
|---|---|---|---|---|---|---|---|
| | | average % methylation | | | % methylation increase | | |
| CpG island | n CpGs | increase after ischemia | p value | FDR value | with cold ischemia time (h) | p value | FDR value |
| chr1: 152008838-152009112 | 21 | 0.91 | 0.00122584 | 0.014203 | 0.99 | 1.77E−05 | 0.009603725 |
| chr1: 156877769-156878649 | 11 | 0.92 | 0.00534559 | 0.038574 | 0.87 | 0.00025 | 0.040838476 |
| chr1: 16085147-16085862 | 25 | 1.33 | 7.12E−05 | 0.00211 | 0.87 | 1.92E−05 | 0.009716415 |
| chr1: 19970255-19971923 | 34 | 1.29 | 6.02E−11 | 3.65E−08 | 0.59 | 8.66E−05 | 0.022781923 |
| chr1: 32169537-32169869 | 19 | 1.42 | 6.03E−11 | 3.65E−08 | 0.75 | 9.86E−05 | 0.025175867 |
| chr2: 27579296-27580135 | 18 | 0.30 | 0.00194542 | 0.019392 | 0.75 | 0.0002 | 0.036716868 |
| chr2: 66672431-66673636 | 21 | 1.89 | 8.55E−15 | 2.47E−11 | 0.82 | 2.57E−06 | 0.002231103 |
| chr2: 74781494-74782685 | 26 | 0.54 | 0.00626869 | 0.042868 | 0.6 | 0.00016 | 0.032221072 |
| chr2: 85640969-85641259 | 25 | 1.29 | 0.00012104 | 0.00311 | 1.14 | 1.89E−05 | 0.009716415 |
| chr2: 85980499-85982198 | 23 | 0.50 | 0.00165714 | 0.017397 | 0.86 | 2.46E−05 | 0.011240042 |
| chr3: 128205495-128212274 | 44 | 0.66 | 2.92E−05 | 0.001127 | 0.54 | 3.50E−05 | 0.013468469 |
| chr3: 146187108-146187710 | 10 | 1.73 | 3.93E−05 | 0.001396 | 2.22 | 3.38E−07 | 0.00055018 |
| chr3: 170136242-170137886 | 21 | 1.03 | 4.65E−09 | 1.35E−06 | 0.83 | 0.00013 | 0.028633263 |
| chr3: 44802852-44803618 | 18 | 0.80 | 2.64E−05 | 0.001056 | 1.39 | 7.74E−06 | 0.00559946 |
| chr4: 4864456-4864834 | 18 | 0.70 | 0.00120177 | 0.014012 | 0.66 | 0.0003 | 0.045570045 |
| chr4: 79472806-79473177 | 14 | 1.26 | 0.00703026 | 0.04624 | 0.94 | 0.00019 | 0.035626493 |
| chr5: 150051116-150052107 | 17 | 0.90 | 0.00286004 | 0.025269 | 1.14 | 0.0003 | 0.045570045 |
| chr6: 10882926-10883149 | 14 | 0.62 | 0.00221371 | 0.02112 | 0.93 | 1.73E−05 | 0.009586409 |
| chr6: 30852102-30852676 | 64 | 1.79 | 1.53E−28 | 3.99E−24 | 0.96 | 1.63E−11 | 1.06E−07 |
| chr6: 32121829-32122529 | 81 | 1.17 | 5.75E−15 | 2.14E−11 | 0.6 | 4.15E−07 | 0.000568856 |
| chr6: 33244677-33245554 | 71 | 1.26 | 1.13E−11 | 1.05E−08 | 0.97 | 1.05E−06 | 0.001093848 |
| chr6: 37503538-37504291 | 15 | 1.57 | 3.54E−05 | 0.001295 | 2.59 | 4.20E−11 | 2.19E−07 |
| chr6: 44187186-44187400 | 18 | 0.93 | 5.93E−06 | 0.000326 | 0.8091776 | 0.00012 | 0.027156853 |
| chr6: 56818873-56820308 | 16 | 0.40 | 0.00666901 | 0.04463 | 1.0201249 | 8.48E−06 | 0.005849883 |
| chr7: 120969587-120970743 | 18 | 0.71 | 0.0011074 | 0.013341 | 0.7142528 | 0.00018 | 0.035284567 |
| chr7: 27190274-27191115 | 24 | 1.06 | 4.54E−05 | 0.001554 | 1.0070883 | 6.27E−08 | 0.00013608 |
| chr7: 63505977-63506298 | 8 | 2.18 | 3.01E−06 | 0.000195 | 2.3619374 | 0.00011 | 0.026372154 |
| chr8: 41165852-41167140 | 29 | 0.72 | 0.00178539 | 0.018378 | 0.5925679 | 0.00022 | 0.039063184 |
| chr9: 1050078-1050510 | 16 | 0.75 | 7.80E−05 | 0.00226 | 0.806199 | 0.00026 | 0.042356651 |
| chr10: 116163391-116164599 | 19 | 1.04 | 0.00553637 | 0.039442 | 0.8192897 | 3.43E−05 | 0.013468469 |
| chr10: 8091374-8098329 | 65 | 0.46 | 2.82E−07 | 3.53E−05 | 0.5430113 | 1.94E−05 | 0.009716415 |
| chr11: 119186947-119187894 | 20 | 0.64 | 0.00237302 | 0.022224 | 0.656425 | 0.00019 | 0.035573633 |
| chr11: 65325081-65326209 | 16 | 0.60 | 0.00070176 | 0.009896 | 1.1010865 | 4.75E−05 | 0.01507488 |
| chr11: 79148358-79152200 | 30 | 0.49 | 0.00012542 | 0.003196 | 0.9617349 | 4.62E−05 | 0.01504041 |
| chr11: 94706291-94707060 | 20 | 0.42 | 0.00390344 | 0.031222 | 1.1275783 | 0.00022 | 0.039063184 |
| chr12: 49738680-49740841 | 20 | 0.12 | 0.00390545 | 0.031222 | 1.0935042 | 0.0002 | 0.03655387 |
| chr12: 57609976-57611168 | 24 | 0.40 | 0.00340812 | 0.028497 | 0.7137577 | 0.00018 | 0.035038325 |
| chr13: 50697984-50702286 | 19 | 0.43 | 0.00020289 | 0.004378 | 0.8704022 | 0.00023 | 0.039262947 |
| chr14: 61746804-61748141 | 17 | 1.93 | 1.03E−07 | 1.67E−05 | 1.1402569 | 1.68E−05 | 0.009511722 |
| chr14: 61787880-61789467 | 28 | 1.43 | 2.59E−08 | 5.72E−06 | 0.8035012 | 0.00012 | 0.027784065 |
| chr15: 101389732-101390260 | 16 | 0.91 | 0.00026046 | 0.023389 | 2.3389174 | 1.17E−08 | 3.81E−05 |
| chr15: 41217789-41223180 | 31 | 0.64 | 0.00013483 | 0.003357 | 0.5012385 | 4.12E−05 | 0.014902956 |
| chr15: 71407656-71408498 | 21 | 0.69 | 0.00013107 | 0.003298 | 0.8682475 | 8.76E−06 | 0.005849883 |
| chr15: 72522131-72524238 | 29 | 1.13 | 0.00035451 | 0.006359 | 0.6266084 | 0.00018 | 0.035497007 |
| chr15: 74218696-74220373 | 33 | 1.34 | 3.02E−10 | 1.46E−07 | 0.6815409 | 0.00011 | 0.026750082 |
| chr16: 66958733-66959655 | 17 | 1.15 | 0.00173637 | 0.01804 | 0.8879075 | 0.00014 | 0.029940678 |
| chr16: 68298012-68298979 | 18 | 1.03 | 1.41E−06 | 0.000111 | 1.0020001 | 5.27E−05 | 0.015776078 |
| chr16: 86539118-86539486 | 10 | 1.20 | 4.45E−05 | 0.011876 | 1.1875647 | 0.00029 | 0.045031324 |
| chr17: 14204168-14207702 | 31 | 0.69 | 8.10E−07 | 7.43E−05 | 0.5653712 | 6.16E−05 | 0.017658404 |
| chr17: 1952919-1962328 | 84 | 0.17 | 0.00155283 | 0.016624 | 0.8775565 | 1.03E−14 | 2.68E−10 |
| chr17: 26925742-26926512 | 16 | 0.98 | 0.00037669 | 0.006607 | 1.2783942 | 0.00011 | 0.026803816 |
| chr17: 48585385-48586167 | 18 | 1.20 | 1.16E−05 | 0.000554 | 1.9663043 | 2.12E−12 | 2.76E−08 |
| chr17: 48636103-48639279 | 46 | 0.38 | 0.0030409 | 0.026392 | 0.4479831 | 0.00013 | 0.029074653 |
| chr17: 74706465-74707067 | 15 | 1.01 | 0.00027218 | 0.005306 | 1.1080732 | 1.37E−05 | 0.00829774 |
| chr18: 24126780-24131138 | 36 | 0.68 | 7.13E−06 | 0.00038 | 0.6478899 | 9.55E−05 | 0.024625762 |
| chr18: 30349690-30352302 | 25 | 1.10 | 8.35E−08 | 1.40E−05 | 0.9334036 | 5.34E−09 | 1.99E−05 |
| chr19: 1465206-1471241 | 21 | 0.68 | 0.00033055 | 0.006055 | 1.3128307 | 9.50E−08 | 0.000190322 |
| chr19: 34012271-34012936 | 17 | 0.55 | 0.00119936 | 0.014012 | 0.8538192 | 0.00011 | 0.026750082 |
| chr19: 46916587-46916862 | 11 | 1.15 | 0.00134375 | 0.01506 | 1.6254384 | 0.00014 | 0.029372897 |
| chr19: 47922251-47922777 | 17 | 0.58 | 0.00093566 | 0.011923 | 0.7974177 | 2.32E−05 | 0.010789657 |
| chr19: 496158-496481 | 10 | 0.69 | 0.00205732 | 0.020137 | 1.1464837 | 5.68E−05 | 0.016621339 |
| chr19: 50931270-50931638 | 9 | 2.11 | 0.00028658 | 0.005505 | 2.1457641 | 2.55E−05 | 0.011256305 |
| chr20: 37230523-37230742 | 12 | 1.09 | 4.31E−05 | 0.001493 | 1.4610661 | 1.43E−05 | 0.0084643 |
| chr21: 34395128-34400245 | 34 | 0.44 | 2.17E−06 | 0.000153 | 0.5756563 | 0.00025 | 0.040838476 |
| chr21: 46785130-46785339 | 10 | 1.26 | 0.00030982 | 0.005764 | 1.1522913 | 0.0003 | 0.045570045 |
| chr22: 32339933-32341192 | 29 | 1.12 | 1.29E−09 | 4.98E−07 | 0.9340402 | 4.74E−06 | 0.003857768 |

TABLE 6
List of CpGs and annotation for the methylated CpGs (ischemia-induced) used in the 1000 minimal LASSO models.

| | No of | | | | | | | |
|---|---|---|---|---|---|---|---|---|
| | times | | | | | | | |
| CpG | used | Percentage | chr | pos | strand | Islands_Name | Relation_to_Island | UCSC_RefGene_Name |
| cg01811187 | 767 | 76.70% | chr17 | 48637445 | + | chr17: 48636103-48639279 | Island | CACNA1G |
| cg17078427 | 703 | 70.30% | chr3 | 170137552 | − | chr3: 170136242-170137886 | Island | CLDN11 |
| cg16547027 | 462 | 46.20% | chr18 | 24127588 | − | chr18: 24126780-24131138 | Island | KCTD1 |
| cg19596468 | 458 | 45.80% | chr4 | 4864110 | + | chr4: 4864456-4864834 | N_Shore | MSX1 |
| cg14309111 | 430 | 43.00% | chr11 | 79150411 | + | chr11: 79148358-79152200 | Island | ODZ4 |
| cg17603502 | 415 | 41.50% | chr17 | 14204056 | − | chr17: 14204168-14207702 | N_Shore | HS3ST3B1 |
| cg08133931 | 384 | 38.40% | chr17 | 48636626 | + | chr17: 48636103-48639279 | Island | |
| cg18599069 | 342 | 34.20% | chr10 | 8096991 | + | chr10: 8091374-8098329 | Island | GATA3 |
| cg24840099 | 239 | 23.90% | chr4 | 4864430 | + | chr4: 4864456-4864834 | N_Shore | MSX1 |
| cg09529433 | 220 | 22.00% | chr17 | 48637255 | + | chr17: 48636103-48639279 | Island | CACNA1G |
| cg10096645 | 220 | 22.00% | chr18 | 24130851 | + | chr18: 24126780-24131138 | Island | KCTD1 |
| cg06108383 | 211 | 21.10% | chr6 | 32120899 | − | chr6: 32121829-32122529 | N_Shore | PPT2; PRRT1 |
| cg03884082 | 172 | 17.20% | chr1 | 19971709 | + | chr1: 19970255-19971923 | Island | NBL1 |
| cg01065003 | 171 | 17.10% | chr18 | 24130839 | − | chr18: 24126780-24131138 | Island | KCTD1 |
| cg22647713 | 168 | 16.80% | chr10 | 8095697 | − | chr10: 8091374-8098329 | Island | FLJ45983; GATA3 |
| cg20449692 | 162 | 16.20% | chr3 | 170136920 | − | chr3: 170136242-170137886 | Island | CLDN11 |
| cg07136023 | 150 | 15.00% | chr16 | 86537316 | − | chr16: 86539118-86539486 | N_Shore | |
| cg20811659 | 136 | 13.60% | chr17 | 48637730 | − | chr17: 48636103-48639279 | Island | CACNA1G |
| cg20048434 | 132 | 13.20% | chr10 | 116163160 | − | chr10: 116163391-116164599 | N_Shore | AFAP1L2 |
| cg06546607 | 127 | 12.70% | chr19 | 34013019 | + | chr19: 34012271-34012936 | S_Shore | PEPD |
| cg00403498 | 127 | 12.70% | chr6 | 32119923 | − | chr6: 32121829-32122529 | N_Shore | PRRT1; PPT2 |
| cg20891301 | 119 | 11.90% | chr4 | 4864711 | − | chr4: 4864456-4864834 | Island | MSX1 |
| cg17416730 | 116 | 11.60% | chr6 | 33245541 | − | chr6: 33244677-33245554 | Island | B3GALT4 |
| cg01724566 | 113 | 11.30% | chr17 | 26926132 | + | chr17: 26925742-26926512 | Island | SPAG5 |
| cg16501308 | 112 | 11.20% | chr18 | 30350221 | − | chr18: 30349690-30352302 | Island | KLHL14 |
| cg06230736 | 109 | 10.90% | chr10 | 8096650 | + | chr10: 8091374-8098329 | Island | FLJ45983; GATA3 |
| cg03199651 | 105 | 10.50% | chr4 | 4862770 | − | chr4: 4864456-4864834 | N_Shore | MSX1 |
| cg06329022 | 103 | 10.30% | chr17 | 26926511 | + | chr17: 26925742-26926512 | Island | SPAG5 |
| cg13879776 | 102 | 10.20% | chr3 | 170136263 | − | chr3: 170136242-170137886 | Island | CLDN11 |
| cg09024124 | 97 | 9.70% | chr3 | 128207255 | − | chr3: 128205495-128212274 | Island | GATA2 |
| cg01507046 | 96 | 9.60% | chr17 | 48637818 | − | chr17: 48636103-48639279 | Island | CACNA1G |
| cg17113856 | 96 | 9.60% | chr6 | 32120895 | − | chr6: 32121829-32122529 | N_Shore | PPT2; PRRT1 |
| cg07846167 | 94 | 9.40% | chr1 | 16084758 | − | chr1: 16085147-16085862 | N_Shore | FBLIM1 |
| cg18701660 | 85 | 8.50% | chr19 | 34012935 | − | chr19: 34012271-34012936 | Island | PEPD |
| cg07516470 | 82 | 8.20% | chr10 | 8095651 | − | chr10: 8091374-8098329 | Island | FLJ45983; GATA3 |
| cg21096399 | 82 | 8.20% | chr11 | 119188145 | + | chr11: 119186947-119187894 | S_Shore | MCAM |
| cg18187680 | 77 | 7.70% | chr10 | 8095825 | − | chr10: 8091374-8098329 | Island | FLJ45983; GATA3 |
| cg16519300 | 76 | 7.60% | chr1 | 16084830 | − | chr1: 16085147-16085862 | N_Shore | FBLIM1 |
| cg06375949 | 75 | 7.50% | chr4 | 4863356 | − | chr4: 4864456-4864834 | N_Shore | MSX1 |
| cg22590761 | 73 | 7.30% | chr15 | 74218921 | + | chr15: 74218696-74220373 | Island | LOXL1 |
| cg26292521 | 70 | 7.00% | chr10 | 8095831 | − | chr10: 8091374-8098329 | Island | FLJ45983; GATA3 |
| cg00110832 | 69 | 6.90% | chr6 | 32121130 | − | chr6: 32121829-32122529 | N_Shore | PPT2PRRT1 |
| cg04255616 | 67 | 6.70% | chr8 | 41167278 | + | chr8: 41165852-41167140 | S_Shore | SFRP1 |
| cg27426707 | 67 | 6.70% | chr17 | 48639585 | + | chr17: 48636103-48639279 | S_Shore | CACNA1G |
| cg24605046 | 66 | 6.60% | chr6 | 33245895 | − | chr6: 33244677-33245554 | S_Shore | B3GALT4 |
| cg12883279 | 62 | 6.20% | chr6 | 32120773 | + | chr6: 32121829-32122529 | N_Shore | PPT2; PRRT1 |
| cg18454685 | 62 | 6.20% | chr17 | 48639239 | + | chr17: 48636103-48639279 | Island | CACNA1G |
| cg25426302 | 62 | 6.20% | chr6 | 32120826 | − | chr6: 32121829-32122529 | N_Shore | PPT2; PRRT1 |
| cg16650717 | 61 | 6.10% | chr1 | 19970334 | − | chr1: 19970255-19971923 | Island | NBL1 |
| cg26270195 | 61 | 6.10% | chr6 | 33245553 | − | chr6: 33244677-33245554 | Island | B3GALT4 |
| cg00449941 | 60 | 6.00% | chr17 | 26926011 | + | chr17: 26925742-26926512 | Island | SPAG5 |
| cg23058185 | 60 | 6.00% | chr10 | 8095985 | − | chr10: 8091374-8098329 | Island | FLJ45983; GATA3 |
| cg03970849 | 59 | 5.90% | chr11 | 79148183 | − | chr11: 79148358-79152200 | N_Shore | ODZ4 |
| cg09998861 | 58 | 5.80% | chr16 | 86538106 | − | chr16: 86539118-86539486 | N_Shore | |
| cg19315863 | 56 | 5.60% | chr10 | 8096597 | + | chr10: 8091374-8098329 | Island | FLJ45983; GATA3 |
| cg17960080 | 55 | 5.50% | chr17 | 26926868 | − | chr17: 26925742-26926512 | S_Shore | SPAG5 |
| cg12163955 | 53 | 5.30% | chr15 | 41217556 | − | chr15: 41217789-41223180 | N_Shore | |
| cg06206801 | 52 | 5.20% | chr18 | 24131379 | − | chr18: 24126780-24131138 | S_Shore | KCTD1 |
| cg06803850 | 51 | 5.10% | chr17 | 26926738 | + | chr17: 26925742-26926512 | S_Shore | SPAG5 |
| cg10049535 | 51 | 5.10% | chr16 | 68299128 | − | chr16: 68298012-68298979 | S_Shore | SLC7A6 |
| cg14098681 | 50 | 5.00% | chr10 | 8096818 | − | chr10: 8091374-8098329 | Island | FLJ45983; GATA3 |
| cg20652404 | 49 | 4.90% | chr15 | 74218904 | + | chr15: 74218696-74220373 | Island | LOXL1 |
| cg08238215 | 47 | 4.70% | chr2 | 66673985 | − | chr2: 66672431-66673636 | S_Shore | MEIS1 |
| cg13934406 | 47 | 4.70% | chr6 | 32120878 | + | chr6: 32121829-32122529 | N_Shore | PPT2; PRRT1 |
| cg25144207 | 47 | 4.70% | chr4 | 4864302 | + | chr4: 4864456-4864834 | N_Shore | MSX1 |
| cg25755953 | 47 | 4.70% | chr17 | 26926457 | − | chr17: 26925742-26926512 | Island | SPAG5 |
| cg24329557 | 45 | 4.50% | chr6 | 10882326 | − | chr6: 10882926-10883149 | N_Shore | GCM2 |
| cg00319655 | 43 | 4.30% | chr4 | 79473327 | − | chr4: 79472806-79473177 | S_Shore | ANXA3 |
| cg03189210 | 41 | 4.10% | chr6 | 33245474 | − | chr6: 33244677-33245554 | Island | B3GALT4 |
| cg04963480 | 40 | 4.00% | chr15 | 71408776 | + | chr15: 71407656-71408498 | S_Shore | CT62 |
| cg04262471 | 38 | 3.80% | chr6 | 33245585 | + | chr6: 33244677-33245554 | S_Shore | B3GALT4 |
| cg17182507 | 38 | 3.80% | chr17 | 1957231 | − | chr17: 1952919-1962328 | Island | HIC1 |
| cg02048416 | 37 | 3.70% | chr2 | 74782684 | + | chr2: 74781494-74782685 | Island | DOK1 |
| cg07346931 | 37 | 3.70% | chr12 | 49743523 | − | chr12: 49738680-49740841 | S_Shelf | DNAJC22 |
| cg20328456 | 37 | 3.70% | chr6 | 32121113 | − | chr6: 32121829-32122529 | N_Shore | PPT2; PRRT1 |
| cg06023994 | 36 | 3.60% | chr3 | 170137871 | + | chr3: 170136242-170137886 | Island | CLDN11 |
| cg07434518 | 36 | 3.60% | chr3 | 170136327 | + | chr3: 170136242-170137886 | Island | CLDN11 |
| cg11590420 | 36 | 3.60% | chr5 | 150051566 | − | chr5: 150051116-150052107 | Island | MYOZ3 |
| cg14176930 | 36 | 3.60% | chr6 | 10884891 | + | chr6: 10882926-10883149 | S_Shore | |
| cg15520477 | 36 | 3.60% | chr19 | 34012957 | − | chr19: 34012271-34012936 | S_Shore | PEPD |
| cg04749507 | 33 | 3.30% | chr6 | 32120203 | + | chr6: 32121829-32122529 | N_Shore | PPT2; PRRT1 |
| cg08062469 | 33 | 3.30% | chr17 | 26926627 | + | chr17: 26925742-26926512 | S_Shore | SPAG5 |
| cg12741994 | 33 | 3.30% | chr3 | 170137321 | + | chr3: 170136242-170137886 | Island | CLDN11 |
| cg19679989 | 33 | 3.30% | chr10 | 8096602 | + | chr10: 8091374-8098329 | Island | FLJ45983; GATA3 |
| cg20663200 | 33 | 3.30% | chr10 | 116163392 | − | chr10: 116163391-116164599 | Island | AFAP1L2 |
| cg23943136 | 32 | 3.20% | chr10 | 8095755 | − | chr10: 8091374-8098329 | Island | FLJ45983; GATA3 |
| cg13398291 | 31 | 3.10% | chr8 | 41166169 | − | chr8: 41165852-41167140 | Island | SFRP1 |
| cg14315444 | 31 | 3.10% | chr17 | 48636344 | − | chr17: 48636103-48639279 | Island | |
| cg23520930 | 31 | 3.10% | chr3 | 128206967 | + | chr3: 128205495-128212274 | Island | GATA2 |
| cg03682712 | 30 | 3.00% | chr15 | 74219307 | − | chr15: 74218696-74220373 | Island | LOXL1 |
| cg22880620 | 30 | 3.00% | chr6 | 56820808 | + | chr6: 56818873-56820308 | S_Shore | BEND6; DST |
| cg25987744 | 30 | 3.00% | chr19 | 46916588 | − | chr19: 46916587-46916862 | Island | CCDC8; CCDC8 |
| cg26381352 | 30 | 3.00% | chr6 | 33244799 | − | chr6: 33244677-33245554 | Island | B3GALT4 |
| cg02551743 | 29 | 2.90% | chr2 | 66673428 | − | chr2: 66672431-66673636 | Island | MEIS1 |
| cg11522683 | 29 | 2.90% | chr6 | 37501428 | + | chr6: 37503538-37504291 | N_Shelf | |
| cg02989257 | 28 | 2.80% | chr1 | 32169274 | − | chr1: 32169537-32169869 | N_Shore | COL16A1 |
| cg08707112 | 28 | 2.80% | chr10 | 8095764 | + | chr10: 8091374-8098329 | Island | FLJ45983; GATA3 |
| cg14327531 | 28 | 2.80% | chr10 | 8097331 | − | chr10: 8091374-8098329 | Island | GATA3 |
| cg23359665 | 28 | 2.80% | chr6 | 32120907 | − | chr6: 32121829-32122529 | N_Shore | PPT2; PRRT1 |
| cg00868875 | 27 | 2.70% | chr18 | 24127237 | − | chr18: 24126780-24131138 | Island | KCTD1 |
| cg21785145 | 27 | 2.70% | chr17 | 48635853 | + | chr17: 48636103-48639279 | N_Shore | |
| cg11129609 | 26 | 2.60% | chr6 | 33247250 | − | chr6: 33244677-33245554 | S_Shore | WDR46 |
| cg17566118 | 26 | 2.60% | chr10 | 8095797 | + | chr10: 8091374-8098329 | Island | FLJ45983; GATA3 |
| cg02241055 | 24 | 2.40% | chr3 | 170136766 | + | chr3: 170136242-170137886 | Island | CLDN11 |
| cg05942574 | 24 | 2.40% | chr17 | 48637104 | − | chr17: 48636103-48639279 | Island | CACNA1G |
| cg10074727 | 24 | 2.40% | chr6 | 10883105 | − | chr6: 10882926-10883149 | Island | GCM2 |
| cg01803928 | 22 | 2.20% | chr13 | 50701619 | + | chr13: 50697984-50702286 | Island | |
| cg05671070 | 22 | 2.20% | chr10 | 8095960 | − | chr10: 8091374-8098329 | Island | FLJ45983; GATA3 |
| cg12064947 | 22 | 2.20% | chr15 | 41220983 | − | chr15: 41217789-41223180 | Island | DLL4 |
| cg12730771 | 22 | 2.20% | chr10 | 8095996 | − | chr10: 8091374-8098329 | Island | FLJ45983; GATA3 |
| cg24509300 | 22 | 2.20% | chr6 | 32123034 | − | chr6: 32121829-32122529 | S_Shore | PPT2 |
| cg00086577 | 21 | 2.10% | chr6 | 32122894 | + | chr6: 32121829-32122529 | S_Shore | PPT2 |
| cg11386011 | 21 | 2.10% | chr6 | 32121156 | + | chr6: 32121829-32122529 | N_Shore | PPT2; PRRT1 |
| cg01111041 | 20 | 2.00% | chr6 | 32121055 | + | chr6: 32121829-32122529 | N_Shore | PPT2; PRRT1 |
| cg04164190 | 20 | 2.00% | chr17 | 14205456 | − | chr17: 14204168-14207702 | Island | HS3ST3B1 |
| cg07841173 | 20 | 2.00% | chr3 | 128210150 | − | chr3: 128205495-128212274 | Island | GATA2 |
| cg19657198 | 20 | 2.00% | chr10 | 8095121 | − | chr10: 8091374-8098329 | Island | FLJ45983 |
| cg20155566 | 20 | 2.00% | chr17 | 26926074 | − | chr17: 26925742-26926512 | Island | SPAG5 |
| cg23104954 | 20 | 2.00% | chr13 | 50701501 | + | chr13: 50697984-50702286 | Island | |
| cg02344539 | 19 | 1.90% | chr17 | 48637743 | + | chr17: 48636103-48639279 | Island | CACNA1G |
| cg11731114 | 19 | 1.90% | chr10 | 8096064 | + | chr10: 8091374-8098329 | Island | FLJ45983; GATA3 |
| cg03696345 | 18 | 1.80% | chr21 | 34398114 | + | chr21: 34395128-34400245 | Island | OLIG2 |
| cg04186868 | 18 | 1.80% | chr12 | 57611144 | − | chr12: 57609976-57611168 | Island | NXPH4 |
| cg07060913 | 18 | 1.80% | chr16 | 86537142 | + | chr16: 86539118-86539486 | N_Shore | |
| cg09573795 | 18 | 1.80% | chr4 | 4863874 | + | chr4: 4864456-4864834 | N_Shore | MSX1 |
| cg19882268 | 18 | 1.80% | chr6 | 33245779 | − | chr6: 33244677-33245554 | S_Shore | B3GALT4 |
| cg20654074 | 18 | 1.80% | chr15 | 41223179 | + | chr15: 41217789-41223180 | Island | DLL4 |
| cg02503117 | 17 | 1.70% | chr16 | 86538424 | − | chr16: 86539118-86539486 | N_Shore | |
| cg08076158 | 17 | 1.70% | chr16 | 86539022 | − | chr16: 86539118-86539486 | N_Shore | |
| cg12626589 | 17 | 1.70% | chr6 | 32120783 | + | chr6: 32121829-32122529 | N_Shore | PPT2; PRRT1; PPT2 |
| cg13484546 | 15 | 1.50% | chr1 | 16084939 | − | chr1: 16085147-16085862 | N_Shore | FBLIM1 |
| cg14261472 | 15 | 1.50% | chr17 | 48637449 | + | chr17: 48636103-48639279 | Island | CACNA1G |
| cg14294793 | 15 | 1.50% | chr11 | 79150593 | + | chr11: 79148358-79152200 | Island | ODZ4 |
| cg15330117 | 15 | 1.50% | chr10 | 8096669 | − | chr10: 8091374-8098329 | Island | FLJ45983; GATA3 |
| cg17991695 | 15 | 1.50% | chr6 | 10882974 | + | chr6: 10882926-10883149 | Island | GCM2 |
| cg02694099 | 14 | 1.40% | chr15 | 71408914 | − | chr15: 71407656-71408498 | S_Shore | CT62 |
| cg11071401 | 14 | 1.40% | chr17 | 48637194 | + | chr17: 48636103-48639279 | Island | CACNA1G |
| cg15472071 | 14 | 1.40% | chr1 | 16085984 | + | chr1: 16085147-16085862 | S_Shore | FBLIM1 |
| cg08306084 | 13 | 1.30% | chr6 | 33248546 | − | chr6: 33244677-33245554 | S_Shelf | WDR46 |
| cg13882090 | 13 | 1.30% | chr6 | 33246094 | + | chr6: 33244677-33245554 | S_Shore | B3GALT4 |
| cg16662821 | 13 | 1.30% | chr8 | 41164679 | − | chr8: 41165852-41167140 | N_Shore | SFRP1 |
| cg19814946 | 13 | 1.30% | chr17 | 14205248 | − | chr17: 14204168-14207702 | Island | HS3ST3B1 |
| cg01546243 | 12 | 1.20% | chr14 | 61748019 | + | chr14: 61746804-61748141 | Island | TMEM30B |
| cg01626459 | 12 | 1.20% | chr6 | 56820778 | − | chr6: 56818873-56820308 | S_Shore | BEND6; DST |
| cg04216597 | 12 | 1.20% | chr17 | 48639836 | + | chr17: 48636103-48639279 | S_Shore | CACNA1G |
| cg07147364 | 12 | 1.20% | chr1 | 19970256 | − | chr1: 19970255-19971923 | Island | NBL1 |
| cg11303127 | 12 | 1.20% | chr12 | 49740807 | + | chr12: 49738680-49740841 | Island | DNAJC22 |
| cg11950383 | 12 | 1.20% | chr21 | 34400072 | − | chr21: 34395128-34400245 | Island | OLIG2 |
| cg16481280 | 12 | 1.20% | chr6 | 32120955 | + | chr6: 32121829-32122529 | N_Shore | PPT2; PRRT1 |
| cg19333963 | 12 | 1.20% | chr19 | 1467979 | + | chr19: 1465206-1471241 | Island | APC2 |
| cg21333861 | 12 | 1.20% | chr6 | 33244976 | − | chr6: 33244677-33245554 | Island | B3GALT4 |
| cg04641787 | 11 | 1.10% | chr10 | 8096154 | − | chr10: 8091374-8098329 | Island | FLJ45983; GATA3 |
| cg05620923 | 11 | 1.10% | chr19 | 1466647 | − | chr19: 1465206-1471241 | Island | APC2 |
| cg06018514 | 11 | 1.10% | chr15 | 41219741 | − | chr15: 41217789-41223180 | Island | |
| cg06133205 | 11 | 1.10% | chr13 | 50701960 | − | chr13: 50697984-50702286 | Island | |
| cg09255732 | 11 | 1.10% | chr1 | 32171050 | − | chr1: 32169537-32169869 | S_Shore | COL16A1 |
| cg09337254 | 11 | 1.10% | chr2 | 85640762 | + | chr2: 85640969-85641259 | N_Shore | |
| cg14040722 | 11 | 1.10% | chr20 | 37229509 | − | chr20: 37230523-37230742 | N_Shore | C20orf95 |
| cg15187550 | 11 | 1.10% | chr10 | 8096370 | − | chr10: 8091374-8098329 | Island | FLJ45983; GATA3 |
| cg16553500 | 11 | 1.10% | chr1 | 32169868 | + | chr1: 32169537-32169869 | Island | COL16A1 |
| cg18923740 | 11 | 1.10% | chr1 | 19971790 | − | chr1: 19970255-19971923 | Island | NBL1 |
| cg20682981 | 11 | 1.10% | chr17 | 1962627 | + | chr17: 1952919-1962328 | S_Shore | HIC1 |
| cg21249595 | 11 | 1.10% | chr6 | 30848811 | + | chr6: 30852102-30852676 | N_Shelf | |
| cg27390596 | 11 | 1.10% | chr17 | 48637858 | − | chr17: 48636103-48639279 | Island | CACNA1G |
| cg02962630 | 10 | 1.00% | chr15 | 41222776 | − | chr15: 41217789-41223180 | Island | DLL4 |
| cg10169241 | 10 | 1.00% | chr19 | 1467032 | − | chr19: 1465206-1471241 | Island | APC2 |
| cg12103626 | 10 | 1.00% | chr17 | 14204310 | − | chr17: 14204168-14207702 | Island | HS3ST3B1 |
| cg18932158 | 10 | 1.00% | chr6 | 33248279 | − | chr6: 33244677-33245554 | S_Shelf | WDR46 |
| cg19450714 | 10 | 1.00% | chr17 | 48637584 | + | chr17: 48636103-48639279 | Island | CACNA1G |
| cg01070078 | 9 | 0.90% | chr17 | 1958883 | − | chr17: 1952919-1962328 | Island | HIC1 |
| cg06774283 | 9 | 0.90% | chr17 | 26926076 | − | chr17: 26925742-26926512 | Island | SPAG5 |
| cg06814287 | 9 | 0.90% | chr6 | 32120584 | + | chr6: 32121829-32122529 | N_Shore | PPT2; PRRT1 |
| cg11145160 | 9 | 0.90% | chr3 | 170136278 | − | chr3: 170136242-170137886 | Island | CLDN11 |
| cg14130039 | 9 | 0.90% | chr6 | 32121225 | − | chr6: 32121829-32122529 | N_Shore | PPT2 |
| cg19036075 | 9 | 0.90% | chr15 | 74220295 | + | chr15: 74218696-74220373 | Island | LOXL1 |
| cg21538208 | 9 | 0.90% | chr4 | 4864488 | + | chr4: 4864456-4864834 | Island | MSX1 |
| cg22314314 | 9 | 0.90% | chr3 | 44802754 | − | chr3: 44802852-44803618 | N_Shore | KIF15; KIAA1143 |
| cg22322679 | 9 | 0.90% | chr6 | 33244178 | − | chr6: 33244677-33245554 | N_Shore | B3GALT4; RPS18 |
| cg23010452 | 9 | 0.90% | chr19 | 34013117 | + | chr19: 34012271-34012936 | S_Shore | PEPD |
| cg23047693 | 9 | 0.90% | chr12 | 57608606 | + | chr12: 57609976-57611168 | N_Shore | |
| cg00316759 | 8 | 0.80% | chr15 | 71407484 | − | chr15: 71407656-71408498 | N_Shore | CT62 |
| cg04209727 | 8 | 0.80% | chr18 | 30350441 | − | chr18: 30349690-30352302 | Island | KLHL14 |
| cg04856022 | 8 | 0.80% | chr6 | 32122955 | − | chr6: 32121829-32122529 | S_Shore | PPT2 |
| cg04877280 | 8 | 0.80% | chr6 | 32122738 | − | chr6: 32121829-32122529 | S_Shore | PPT2 |
| cg05945782 | 8 | 0.80% | chr17 | 1954986 | − | chr17: 1952919-1962328 | Island | MIR212 |
| cg26579986 | 8 | 0.80% | chr6 | 37504610 | − | chr6: 37503538-37504291 | S_Shore | |
| cg26704078 | 8 | 0.80% | chr18 | 24131115 | + | chr18: 24126780-24131138 | Island | KCTD1 |
| cg27147350 | 8 | 0.80% | chr6 | 33245881 | − | chr6: 33244677-33245554 | S_Shore | B3GALT4 |
| cg03740978 | 7 | 0.70% | chr18 | 24127875 | − | chr18: 24126780-24131138 | Island | KCTD1 |
| cg03839949 | 7 | 0.70% | chr3 | 128210541 | − | chr3: 128205495-128212274 | Island | GATA2 |
| cg04982951 | 7 | 0.70% | chr10 | 8096635 | + | chr10: 8091374-8098329 | Island | FLJ45983; GATA3 |
| cg05133205 | 7 | 0.70% | chr6 | 32121249 | − | chr6: 32121829-32122529 | N_Shore | PPT2 |
| cg08347183 | 7 | 0.70% | chr10 | 8096633 | + | chr10: 8091374-8098329 | Island | FLJ45983; GATA3 |
| cg10551329 | 7 | 0.70% | chr6 | 32120933 | + | chr6: 32121829-32122529 | N_Shore | PPT2; PRRT1 |
| cg16226644 | 7 | 0.70% | chr6 | 33246091 | − | chr6: 33244677-33245554 | S_Shore | B3GALT4 |
| cg20281962 | 7 | 0.70% | chr10 | 8089733 | − | chr10: 8091374-8098329 | N_Shore | |
| cg20914572 | 7 | 0.70% | chr6 | 32119874 | + | chr6: 32121829-32122529 | N_Shore | PRRT1; PPT2 |
| cg26366048 | 7 | 0.70% | chr6 | 56820386 | − | chr6: 56818873-56820308 | S_Shore | BEND6; DST |
| cg01312445 | 6 | 0.60% | chr16 | 86536684 | − | chr16: 86539118-86539486 | N_Shelf | |
| cg01993576 | 6 | 0.60% | chr6 | 44187674 | + | chr6: 44187186-44187400 | S_Shore | SLC29A1 |
| cg03995156 | 6 | 0.60% | chr6 | 32122864 | + | chr6: 32121829-32122529 | S_Shore | PPT2 |
| cg07555797 | 6 | 0.60% | chr14 | 61788314 | − | chr14: 61787880-61789467 | Island | PRKCH |
| cg09942293 | 6 | 0.60% | chr16 | 66957496 | − | chr16: 66958733-66959655 | N_Shore | RRAD |
| cg10372921 | 6 | 0.60% | chr15 | 74218733 | − | chr15: 74218696-74220373 | Island | LOXL1 |
| cg11941520 | 6 | 0.60% | chr6 | 32121522 | + | chr6: 32121829-32122529 | N_Shore | PPT2 |
| cg16396284 | 6 | 0.60% | chr6 | 33245537 | − | chr6: 33244677-33245554 | Island | B3GALT4 |
| cg16710894 | 6 | 0.60% | chr10 | 8092264 | − | chr10: 8091374-8098329 | Island | |
| cg20161179 | 6 | 0.60% | chr4 | 4863282 | + | chr4: 4864456-4864834 | N_Shore | MSX1 |
| cg24092179 | 6 | 0.60% | chr19 | 50931222 | − | chr19: 50931270-50931638 | N_Shore | SPIB |
| cg00552704 | 5 | 0.50% | chr6 | 32121420 | − | chr6: 32121829-32122529 | N_Shore | PPT2; PPT2 |
| cg05176991 | 5 | 0.50% | chr18 | 24128116 | + | chr18: 24126780-24131138 | Island | KCTD1 |
| cg06902929 | 5 | 0.50% | chr6 | 32123258 | + | chr6: 32121829-32122529 | S_Shore | PPT2; PPT2 |
| cg07273125 | 5 | 0.50% | chr16 | 68295692 | + | chr16: 68298012-68298979 | N_Shelf | |
| cg08483834 | 5 | 0.50% | chr6 | 33248239 | + | chr6: 33244677-33245554 | S_Shelf | WDR46 |
| cg08510658 | 5 | 0.50% | chr6 | 10882927 | − | chr6: 10882926-10883149 | Island | GCM2 |
| cg08890824 | 5 | 0.50% | chr16 | 66958786 | + | chr16: 66958733-66959655 | Island | RRAD |
| cg10094078 | 5 | 0.50% | chr19 | 1467925 | + | chr19: 1465206-1471241 | Island | APC2 |
| cg11215918 | 5 | 0.50% | chr21 | 34395699 | − | chr21: 34395128-34400245 | Island | |
| cg14167596 | 5 | 0.50% | chr4 | 4862910 | − | chr4: 4864456-4864834 | N_Shore | MSX1 |
| cg15852223 | 5 | 0.50% | chr10 | 8096372 | − | chr10: 8091374-8098329 | Island | FLJ45983; GATA3 |
| cg17639046 | 5 | 0.50% | chr17 | 14204027 | − | chr17: 14204168-14207702 | N_Shore | HS3ST3B1 |
| cg19951298 | 5 | 0.50% | chr6 | 10883054 | − | chr6: 10882926-10883149 | Island | GCM2 |
| cg20196291 | 5 | 0.50% | chr10 | 116164849 | − | chr10: 116163391-116164599 | S_Shore | AFAP1L2 |
| cg21973370 | 5 | 0.50% | chr17 | 1957919 | − | chr17: 1952919-1962328 | Island | HIC1 |
| cg22648949 | 5 | 0.50% | chr18 | 30351983 | + | chr18: 30349690-30352302 | Island | KLHL14 |
| cg26784201 | 5 | 0.50% | chr5 | 150050950 | − | chr5: 150051116-150052107 | N_Shore | MYOZ3 |
| cg00360474 | 4 | 0.40% | chr6 | 37504404 | + | chr6: 37503538-37504291 | S_Shore | |
| cg0093O833 | 4 | 0.40% | chr8 | 41168264 | − | chr8: 41165852-41167140 | S_Shore | SFRP1 |
| cg01149449 | 4 | 0.40% | chr11 | 79150906 | + | chr11: 79148358-79152200 | Island | ODZ4 |
| cg02388150 | 4 | 0.40% | chr8 | 41165699 | − | chr8: 41165852-41167140 | N_Shore | SFRP1 |
| cg03718845 | 4 | 0.40% | chr2 | 85640001 | + | chr2: 85640969-85641259 | N_Shore | |
| cg03832440 | 4 | 0.40% | chr17 | 14207241 | + | chr17: 14204168-14207702 | Island | HS3ST3B1; MGC12916 |
| cg04414274 | 4 | 0.40% | chr17 | 1957866 | + | chr17: 1952919-1962328 | Island | HIC1 |
| cg06870728 | 4 | 0.40% | chr10 | 8095363 | − | chr10: 8091374-8098329 | Island | FLJ45983; GATA3 |
| cg07132710 | 4 | 0.40% | chr3 | 128202797 | − | chr3: 128205495-128212274 | N_Shelf | GATA2 |
| cg07306737 | 4 | 0.40% | chr6 | 33247141 | − | chr6: 33244677-33245554 | S_Shore | WDR46 |
| cg09857513 | 4 | 0.40% | chr7 | 120969044 | + | chr7: 120969587-120970743 | N_Shore | WNT16 |
| cg11014463 | 4 | 0.40% | chr6 | 56818479 | − | chr6: 56818873-56820308 | N_Shore | BEND6; DST |
| cg11626629 | 4 | 0.40% | chr6 | 33245460 | − | chr6: 33244677-33245554 | Island | B3GALT4 |
| cg12599673 | 4 | 0.40% | chr15 | 71408847 | − | chr15: 71407656-71408498 | S_Shore | CT62 |
| cg14293300 | 4 | 0.40% | chr21 | 34399361 | + | chr21: 34395128-34400245 | Island | OLIG2 |
| cg14904908 | 4 | 0.40% | chr8 | 41167660 | − | chr8: 41165852-41167140 | S_Shore | SFRP1 |
| cg15140798 | 4 | 0.40% | chr21 | 46782485 | − | chr21: 46785130-46785339 | N_Shelf | |
| cg15839448 | 4 | 0.40% | chr8 | 41166530 | − | chr8: 41165852-41167140 | Island | SFRP1 |
| cg17124583 | 4 | 0.40% | chr10 | 8097641 | − | chr10: 8091374-8098329 | Island | GATA3 |
| cg17764989 | 4 | 0.40% | chr16 | 86539121 | + | chr16: 86539118-86539486 | Island | |
| cg19156220 | 4 | 0.40% | chr6 | 33244752 | − | chr6: 33244677-33245554 | Island | B3GALT4 |
| cg22216643 | 4 | 0.40% | chr17 | 74704158 | − | chr17: 74706465-74707067 | N_Shelf | MXRA7 |
| cg23599559 | 4 | 0.40% | chr17 | 48637438 | − | chr17: 48636103-48639279 | Island | CACNA1G |
| cg24858591 | 4 | 0.40% | chr3 | 44803638 | − | chr3: 44802852-44803618 | S_Shore | KIAA1143; KIF15 |
| cg01160692 | 3 | 0.30% | chr17 | 1959620 | + | chr17: 1952919-1962328 | Island | HIC1 |
| cg01271812 | 3 | 0.30% | chr2 | 66671478 | − | chr2: 66672431-66673636 | N_Shore | MEIS1 |
| cg01626899 | 3 | 0.30% | chr17 | 26925852 | + | chr17: 26925742-26926512 | Island | SPAG5 |
| cg01684248 | 3 | 0.30% | chr16 | 86536239 | − | chr16: 86539118-86539486 | N_Shelf | |
| cg02980693 | 3 | 0.30% | chr3 | 128208970 | + | chr3: 128205495-128212274 | Island | GATA2 |
| cg03306486 | 3 | 0.30% | chr19 | 1467952 | + | chr19: 1465206-1471241 | Island | APC2 |
| cg06022942 | 3 | 0.30% | chr10 | 8095484 | + | chr10: 8091374-8098329 | Island | FLJ45983; GATA3 |
| cg06747432 | 3 | 0.30% | chr19 | 46916741 | + | chr19: 46916587-46916862 | Island | CCDC8 |
| cg06844968 | 3 | 0.30% | chr18 | 24131604 | − | chr18: 24126780-24131138 | S_Shore | KCTD1 |
| cg08438366 | 3 | 0.30% | chr20 | 37230612 | + | chr20: 37230523-37230742 | Island | C20orf95 |
| cg09042577 | 3 | 0.30% | chr11 | 119185584 | − | chr11: 119186947-119187894 | N_Shore | MCAM |
| cg09748975 | 3 | 0.30% | chr4 | 4864532 | + | chr4: 4864456-4864834 | Island | MSX1 |
| cg10464312 | 3 | 0.30% | chr2 | 66672688 | − | chr2: 66672431-66673636 | Island | MEIS1 |
| cg10633838 | 3 | 0.30% | chr6 | 33245359 | + | chr6: 33244677-33245554 | Island | B3GALT4 |
| cg13438549 | 3 | 0.30% | chr17 | 48633206 | + | chr17: 48636103-48639279 | N_Shelf | SPATA20 |
| cg15355859 | 3 | 0.30% | chr11 | 79149352 | − | chr11: 79148358-79152200 | Island | ODZ4 |
| cg15709766 | 3 | 0.30% | chr19 | 1466497 | − | chr19: 1465206-1471241 | Island | APC2 |
| cg17029019 | 3 | 0.30% | chr17 | 1959124 | − | chr17: 1952919-1962328 | Island | HIC1 |
| cg17891011 | 3 | 0.30% | chr10 | 8096152 | − | chr10: 8091374-8098329 | Island | FLJ45983; GATA3 |
| cg18774642 | 3 | 0.30% | chr18 | 30353699 | − | chr18: 30349690-30352302 | S_Shore | KLHL14 |
| cg19241689 | 3 | 0.30% | chr6 | 33245516 | − | chr6: 33244677-33245554 | Island | B3GALT4 |
| cg20706438 | 3 | 0.30% | chr2 | 74783005 | + | chr2: 74781494-74782685 | S_Shore | DOK1 |
| cg21068480 | 3 | 0.30% | chr2 | 85980500 | − | chr2: 85980499-85982198 | Island | ATOH8 |
| cg25520679 | 3 | 0.30% | chr17 | 1959121 | − | chr17: 1952919-1962328 | Island | HIC1 |
| cg26055446 | 3 | 0.30% | chr6 | 33245990 | + | chr6: 33244677-33245554 | S_Shore | B3GALT4 |
| cg00040007 | 2 | 0.20% | chr15 | 41222276 | − | chr15: 41217789-41223180 | Island | DLL4 |
| cg00927777 | 2 | 0.20% | chr17 | 1960199 | − | chr17: 1952919-1962328 | Island | HIC1 |
| cg01616215 | 2 | 0.20% | chr22 | 32340373 | − | chr22: 32339933-32341192 | Island | YWHAH; C22orf24 |
| cg01725608 | 2 | 0.20% | chr7 | 120969666 | − | chr7: 120969587-120970743 | Island | WNT16 |
| cg01785568 | 2 | 0.20% | chr4 | 4864833 | + | chr4: 4864456-4864834 | Island | MSX1 |
| cg01796075 | 2 | 0.20% | chr1 | 156878573 | − | chr1: 156877769-156878649 | Island | PEAR1 |
| cg02956248 | 2 | 0.20% | chr6 | 32120901 | − | chr6: 32121829-32122529 | N_Shore | PPT2; PRRT1; PPT2 |
| cg03814826 | 2 | 0.20% | chr22 | 32341378 | − | chr22: 32339933-32341192 | S_Shore | C22orf24; YWHAH |
| cg04203646 | 2 | 0.20% | chr19 | 1467008 | − | chr19: 1465206-1471241 | Island | APC2 |
| cg04751149 | 2 | 0.20% | chr2 | 66673449 | − | chr2: 66672431-66673636 | Island | MEIS1 |
| cg05003322 | 2 | 0.20% | chr1 | 32169706 | − | chr1: 32169537-32169869 | Island | COL16A1 |
| cg05871997 | 2 | 0.20% | chr6 | 56819623 | − | chr6: 56818873-56820308 | Island | BEND6; DST |
| cg06025456 | 2 | 0.20% | chr6 | 32120863 | + | chr6: 32121829-32122529 | N_Shore | PPT2; PRRT1; PPT2 |
| cg06283368 | 2 | 0.20% | chr15 | 74219669 | + | chr15: 74218696-74220373 | Island | LOXL1 |
| cg12881557 | 2 | 0.20% | chr18 | 24130633 | + | chr18: 24126780-24131138 | Island | KCTD1 |
| cg14250833 | 2 | 0.20% | chr6 | 10882240 | − | chr6: 10882926-10883149 | N_Shore | GCM2 |
| cg14914519 | 2 | 0.20% | chr17 | 14205882 | + | chr17: 14204168-14207702 | Island | HS3ST3B1; MGC12 916 |
| cg16838838 | 2 | 0.20% | chr2 | 85641023 | + | chr2: 85640969-85641259 | Island | |
| cg16868298 | 2 | 0.20% | chr7 | 120969033 | + | chr7: 120969587-120970743 | N_Shore | WNT16 |
| cg17276021 | 2 | 0.20% | chr1 | 16084445 | + | chr1: 16085147-16085862 | N_Shore | FBLIM1 |
| cg17372269 | 2 | 0.20% | chr3 | 44802863 | − | chr3: 44802852-44803618 | Island | KIF15; KIAA1143 |
| cg18374181 | 2 | 0.20% | chr21 | 34401798 | − | chr21: 34395128-34400245 | S_Shore | |
| cg18729787 | 2 | 0.20% | chr6 | 33246307 | + | chr6: 33244677-33245554 | S_Shore | B3GALT4 |
| cg19884965 | 2 | 0.20% | chr11 | 79150305 | − | chr11: 79148358-79152200 | Island | ODZ4 |
| cg20138264 | 2 | 0.20% | chr17 | 48585640 | + | chr17: 48585385-48586167 | Island | MYCBPAP |
| cg20152539 | 2 | 0.20% | chr17 | 14206871 | + | chr17: 14204168-14207702 | Island | HS3ST3B1; MGC12916 |
| cg20180247 | 2 | 0.20% | chr6 | 10884140 | + | chr6: 10882926-10883149 | S_Shore | |
| cg20283670 | 2 | 0.20% | chr10 | 116162728 | − | chr10: 116163391-116164599 | N_Shore | AFAP1L2 |
| cg21435190 | 2 | 0.20% | chr3 | 128208037 | + | chr3: 128205495-128212274 | Island | GATA2 |
| cg23253569 | 2 | 0.20% | chr21 | 34398222 | + | chr21: 34395128-34400245 | Island | OLIG2 |
| cg24399924 | 2 | 0.20% | chr2 | 85980533 | − | chr2: 85980499-85982198 | Island | ATOH8 |
| cg24888989 | 2 | 0.20% | chr3 | 44803291 | − | chr3: 44802852-44803618 | Island | KIF15; KIF15; KIAA1143 |
| cg25075776 | 2 | 0.20% | chr6 | 30848828 | + | chr6: 30852102-30852676 | N_Shelf | |
| cg26418770 | 2 | 0.20% | chr17 | 14206886 | + | chr17: 14204168-14207702 | Island | HS3ST3B1; MGC12916 |
| cg26657382 | 2 | 0.20% | chr16 | 86538510 | − | chr16: 86539118-86539486 | N_Shore | |
| cg26977644 | 2 | 0.20% | chr11 | 79149294 | − | chr11: 79148358-79152200 | Island | ODZ4 |
| cg00183916 | 1 | 0.10% | chr17 | 14204936 | + | chr17: 14204168-14207702 | Island | HS3ST3B1 |
| cg00313401 | 1 | 0.10% | chr15 | 74219948 | + | chr15: 74218696-74220373 | Island | LOXL1 |
| cg00592510 | 1 | 0.10% | chr17 | 1957625 | + | chr17: 1952919-1962328 | Island | HIC1 |
| cg00702638 | 1 | 0.10% | chr3 | 44803293 | − | chr3: 44802852-44803618 | Island | KIF15; KIAA1143 |
| cg00739593 | 1 | 0.10% | chr10 | 116164714 | − | chr10: 116163391-116164599 | S_Shore | AFAP1L2 |
| cg00913604 | 1 | 0.10% | chr16 | 66958650 | − | chr16: 66958733-66959655 | N_Shore | RRAD |
| cg01404873 | 1 | 0.10% | chr13 | 50701050 | + | chr13: 50697984-50702286 | Island | DLEU2 |
| cg01807770 | 1 | 0.10% | chr4 | 79471305 | + | chr4: 79472806-79473177 | N_Shore | ANXA3 |
| cg02151609 | 1 | 0.10% | chr17 | 1957529 | − | chr17: 1952919-1962328 | Island | HIC1 |
| cg02242344 | 1 | 0.10% | chr2 | 85640943 | + | chr2: 85640969-85641259 | N_Shore | |
| cg02339682 | 1 | 0.10% | chr6 | 56819432 | − | chr6: 56818873-56820308 | Island | DST; BEND6 |
| cg02429905 | 1 | 0.10% | chr6 | 32119944 | − | chr6: 32121829-32122529 | N_Shore | PRRT1; PPT2 |
| cg02836487 | 1 | 0.10% | chr3 | 128206457 | − | chr3: 128205495-128212274 | Island | GATA2 |
| cg03133371 | 1 | 0.10% | chr8 | 41167673 | + | chr8: 41165852-41167140 | S_Shore | SFRP1 |
| cg03270204 | 1 | 0.10% | chr6 | 30851638 | − | chr6: 30852102-30852676 | N_Shore | DDR1 |
| cg03356734 | 1 | 0.10% | chr20 | 37230413 | + | chr20: 37230523-37230742 | N_Shore | C20orf95 |
| cg03365354 | 1 | 0.10% | chr11 | 119187391 | − | chr11: 119186947-119187894 | Island | MCAM |
| cg03434432 | 1 | 0.10% | chr6 | 32122393 | − | chr6: 32121829-32122529 | Island | PPT2 |
| cg03570994 | 1 | 0.10% | chr6 | 32121143 | + | chr6: 32121829-32122529 | N_Shore | PPT2; PRRT1 |
| cg03575666 | 1 | 0.10% | chr8 | 41168186 | + | chr8: 41165852-41167140 | S_Shore | SFRP1 |
| cg04105091 | 1 | 0.10% | chr6 | 32121355 | + | chr6: 32121829-32122529 | N_Shore | PPT2 |
| cg04436755 | 1 | 0.10% | chr15 | 74218767 | + | chr15: 74218696-74220373 | Island | LOXL1 |
| cg04852949 | 1 | 0.10% | chr1 | 32170929 | − | chr1: 32169537-32169869 | S_Shore | COL16A1 |
| cg04983516 | 1 | 0.10% | chr11 | 79151719 | + | chr11: 79148358-79152200 | Island | ODZ4 |
| cg05457563 | 1 | 0.10% | chr19 | 1467029 | − | chr19: 1465206-1471241 | Island | APC2 |
| cg05470554 | 1 | 0.10% | chr7 | 120969079 | − | chr7: 120969587-120970743 | N_Shore | WNT16 |
| cg05713782 | 1 | 0.10% | chr11 | 94706830 | − | chr11: 94706291-94707060 | Island | KDM4D; CWC15 |
| cg05946971 | 1 | 0.10% | chr22 | 32341328 | − | chr22: 32339933-32341192 | S_Shore | C22orf24; YWHAH |
| cg06065141 | 1 | 0.10% | chr17 | 1957161 | − | chr17: 1952919-1962328 | Island | HIC1 |
| cg06485671 | 1 | 0.10% | chr18 | 30350935 | − | chr18: 30349690-30352302 | Island | KLHL14 |
| cg06515159 | 1 | 0.10% | chr21 | 34400659 | + | chr21: 34395128-34400245 | S_Shore | OLIG2 |
| cg06642647 | 1 | 0.10% | chr6 | 30848807 | + | chr6: 30852102-30852676 | N_Shelf | |
| cg06892009 | 1 | 0.10% | chr11 | 79151611 | − | chr11: 79148358-79152200 | Island | ODZ4 |
| cg07137845 | 1 | 0.10% | chr3 | 170136485 | − | chr3: 170136242-170137886 | Island | CLDN11 |
| cg07265873 | 1 | 0.10% | chr6 | 30851940 | − | chr6: 30852102-30852676 | N_Shore | DDR1 |
| cg07348922 | 1 | 0.10% | chr6 | 33244990 | + | chr6: 33244677-33245554 | Island | B3GALT4 |
| cg07578663 | 1 | 0.10% | chr10 | 8096600 | + | chr10: 8091374-8098329 | Island | FLJ45983; GATA3; |
| cg08110052 | 1 | 0.10% | chr6 | 32125424 | + | chr6: 32121829-32122529 | S_Shelf | PPT2 |
| cg08509237 | 1 | 0.10% | chr6 | 32122065 | − | chr6: 32121829-32122529 | Island | PPT2 |
| cg08711175 | 1 | 0.10% | chr12 | 57614182 | − | chr12: 57609976-57611168 | S_Shelf | NXPH4 |
| cg09074260 | 1 | 0.10% | chr11 | 94707049 | + | chr11: 94706291-94707060 | Island | KDM4D; CWC15 |
| cg09172659 | 1 | 0.10% | chr17 | 14203711 | + | chr17: 14204168-14207702 | N_Shore | HS3ST3B1 |
| cg09410389 | 1 | 0.10% | chr8 | 41168205 | − | chr8: 41165852-41167140 | S_Shore | SFRP1 |
| cg09535924 | 1 | 0.10% | chr2 | 66671659 | + | chr2: 66672431-66673636 | N_Shore | MEIS1 |
| cg09570958 | 1 | 0.10% | chr17 | 14206774 | − | chr17: 14204168-14207702 | Island | HS3ST3B1; MGC12916 |
| cg09673208 | 1 | 0.10% | chr11 | 79151811 | + | chr11: 79148358-79152200 | Island | ODZ4 |
| cg09829319 | 1 | 0.10% | chr6 | 10882238 | − | chr6: 10882926-10883149 | N_Shore | GCM2 |
| cg10405604 | 1 | 0.10% | chr15 | 101390259 | + | chr15: 101389732-101390260 | Island | |
| cg10541674 | 1 | 0.10% | chr12 | 57610491 | − | chr12: 57609976-57611168 | Island | NXPH4 |
| cg10935762 | 1 | 0.10% | chr3 | 128202176 | + | chr3: 128205495-128212274 | N_Shelf | GATA2 |
| cg10948797 | 1 | 0.10% | chr17 | 1957607 | + | chr17: 1952919-1962328 | Island | HIC1 |
| cg11018337 | 1 | 0.10% | chr10 | 8095495 | + | chr10: 8091374-8098329 | Island | FLJ45983; GATA3 |
| cg11452354 | 1 | 0.10% | chr6 | 44187052 | + | chr6: 44187186-44187400 | N_Shore | SLC29A1 |
| cg11453400 | 1 | 0.10% | chr10 | 116165190 | − | chr10: 116163391-116164599 | S_Shore | AFAP1L2 |
| cg11471939 | 1 | 0.10% | chr15 | 72522966 | + | chr15: 72522131-72524238 | Island | PKM2 |
| cg12280317 | 1 | 0.10% | chr1 | 152008083 | + | chr1: 152008838-152009112 | N_Shore | S100A11 |
| cg12308216 | 1 | 0.10% | chr6 | 30853255 | + | chr6: 30852102-30852676 | S_Shore | DDR1 |
| cg13102294 | 1 | 0.10% | chr6 | 32121393 | − | chr6: 32121829-32122529 | N_Shore | PPT2 |
| cg13161961 | 1 | 0.10% | chr7 | 120970240 | + | chr7: 120969587-120970743 | Island | WNT16 |
| cg13333304 | 1 | 0.10% | chr3 | 170136200 | − | chr3: 170136242-170137886 | N_Shore | CLDN11 |
| cg13365340 | 1 | 0.10% | chr6 | 33245342 | + | chr6: 33244677-33245554 | Island | B3GALT4 |
| cg13431023 | 1 | 0.10% | chr10 | 8096220 | − | chr10: 8091374-8098329 | Island | FLJ45983; GATA3 |
| cg13524919 | 1 | 0.10% | chr21 | 34396506 | + | chr21: 34395128-34400245 | Island | |
| cg13543854 | 1 | 0.10% | chr10 | 8095477 | − | chr10: 8091374-8098329 | Island | FLJ45983; GATA3 |
| cg13793145 | 1 | 0.10% | chr6 | 44187109 | − | chr6: 44187186-44187400 | N_Shore | SLC29A1 |
| cg13915354 | 1 | 0.10% | chr17 | 1957671 | − | chr17: 1952919-1962328 | Island | HIC1 |
| cg13951527 | 1 | 0.10% | chr17 | 1957216 | − | chr17: 1952919-1962328 | Island | HIC1 |
| cg14435807 | 1 | 0.10% | chr15 | 74218780 | + | chr15: 74218696-74220373 | Island | LOXL1 |
| cg14448169 | 1 | 0.10% | chr7 | 120968904 | − | chr7: 120969587-120970743 | N_Shore | WNT16 |
| cg14775296 | 1 | 0.10% | chr2 | 66672841 | − | chr2: 66672431-66673636 | Island | MEIS1 |
| cg14843922 | 1 | 0.10% | chr21 | 34398849 | + | chr21: 34395128-34400245 | Island | OLIG2 |
| cg14950855 | 1 | 0.10% | chr12 | 49740781 | + | chr12: 49738680-49740841 | Island | DNAJC22 |
| cg15543281 | 1 | 0.10% | chr6 | 33245181 | + | chr6: 33244677-33245554 | Island | B3GALT4 |
| cg15657704 | 1 | 0.10% | chr10 | 116164955 | − | chr10: 116163391-116164599 | S_Shore | AFAP1L2 |
| cg15848031 | 1 | 0.10% | chr4 | 4864293 | + | chr4: 4864456-4864834 | N_Shore | MSX1 |
| cg15989091 | 1 | 0.10% | chr2 | 74780172 | + | chr2: 74781494-74782685 | N_Shore | L0XL3 |
| cg16004427 | 1 | 0.10% | chr1 | 16083101 | − | chr1: 16085147-16085862 | N_Shelf | |
| cg16079541 | 1 | 0.10% | chr6 | 30848846 | + | chr6: 30852102-30852676 | N_Shelf | |
| cg16437908 | 1 | 0.10% | chr2 | 85640810 | − | chr2: 85640969-85641259 | N_Shore | |
| cg16477774 | 1 | 0.10% | chr11 | 65325249 | − | chr11: 65325081-65326209 | Island | LTBP3 |
| cg16713743 | 1 | 0.10% | chr21 | 34397135 | + | chr21: 34395128-34400245 | Island | OLIG2 |
| cg18729886 | 1 | 0.10% | chr14 | 61788339 | − | chr14: 61787880-61789467 | Island | PRKCH |
| cg19873719 | 1 | 0.10% | chr6 | 33247107 | + | chr6: 33244677-33245554 | S_Shore | WDR46 |
| cg20457147 | 1 | 0.10% | chr14 | 61787823 | − | chr14: 61787880-61789467 | N_Shore | PRKCH |
| cg20459712 | 1 | 0.10% | chr6 | 56815929 | + | chr6: 56818873-56820308 | N_Shelf | DST |
| cg20731875 | 1 | 0.10% | chr17 | 14207701 | + | chr17: 14204168-14207702 | Island | HS3ST3B1; MGC12916 |
| cg21415424 | 1 | 0.10% | chr6 | 37503074 | + | chr6: 37503538-37504291 | N_Shore | |
| cg22609784 | 1 | 0.10% | chr4 | 4863678 | + | chr4: 4864456-4864834 | N_Shore | MSX1 |
| cg22745102 | 1 | 0.10% | chr19 | 50931616 | + | chr19: 50931270-50931638 | Island | SPIB |
| cg22913903 | 1 | 0.10% | chr12 | 49740968 | − | chr12: 49738680-49740841 | S_Shore | DNAJC22 |
| cg22931738 | 1 | 0.10% | chr3 | 128206823 | + | chr3: 128205495-128212274 | Island | GATA2 |
| cg23305408 | 1 | 0.10% | chr1 | 32169701 | − | chr1: 32169537-32169869 | Island | COL16A1 |
| cg23519308 | 1 | 0.10% | chr19 | 34012901 | − | chr19: 34012271-34012936 | Island | PEPD |
| cg23621097 | 1 | 0.10% | chr17 | 1962236 | + | chr17: 1952919-1962328 | Island | HIC1; HIC1 |
| cg23950233 | 1 | 0.10% | chr6 | 33245739 | − | chr6: 33244677-33245554 | S_Shore | B3GALT4 |
| cg24506025 | 1 | 0.10% | chr11 | 94706874 | + | chr11: 94706291-94707060 | Island | KDM4D; CWC15 |
| cg25161092 | 1 | 0.10% | chr2 | 85638535 | + | chr2: 85640969-85641259 | N_Shelf | CAPG |
| cg25484790 | 1 | 0.10% | chr11 | 119185671 | − | chr11: 119186947-119187894 | N_Shore | MCAM |
| cg26709950 | 1 | 0.10% | chr16 | 66959235 | + | chr16: 66958733-66959655 | Island | RRAD |
| cg27038439 | 1 | 0.10% | chr4 | 4864320 | − | chr4: 4864456-4864834 | N_Shore | MSX1 |
| cg27070869 | 1 | 0.10% | chr6 | 32122779 | − | chr6: 32121829-32122529 | S_Shore | PPT2 |
| cg27357571 | 1 | 0.10% | chr21 | 34398226 | + | chr21: 34395128-34400245 | Island | OLIG2 |

TABLE 7
Lists of CpGs and annotation for the methylated CpGs (ischemia-induced) reoccuring in at least 10% of the minimal LASSO models.

| | No of | | | | | | | |
|---|---|---|---|---|---|---|---|---|
| | times | | | | | | | |
| CpG | used | Percentage | chr | pos | strand | Islands_Name | Relation_to_Island | UCSC_RefGene_Name |
| cg01811187 | 767 | 76.70% | chr17 | 48637445 | + | chr17: 48636103-48639279 | Island | CACNA1G |
| cg17078427 | 703 | 70.30% | chr3 | 170137552 | − | chr3: 170136242-170137886 | Island | CLDN11 |
| cg16547027 | 462 | 46.20% | chr18 | 24127588 | − | chr18: 24126780-24131138 | Island | KCTD1 |
| cg19596468 | 458 | 45.80% | chr4 | 4864110 | + | chr4: 4864456-4864834 | N_Shore | MSX1 |
| cg14309111 | 430 | 43.00% | chr11 | 79150411 | + | chr11: 79148358-79152200 | Island | ODZ4 |
| cg17603502 | 415 | 41.50% | chr17 | 14204056 | − | chr17: 14204168-14207702 | N_Shore | HS3ST3B1 |
| cg08133931 | 384 | 38.40% | chr17 | 48636626 | + | chr17: 48636103-48639279 | Island | |
| cg18599069 | 342 | 34.20% | chr10 | 8096991 | + | chr10: 8091374-8098329 | Island | GATA3 |
| cg24840099 | 239 | 23.90% | chr4 | 4864430 | + | chr4: 4864456-4864834 | N_Shore | MSX1 |
| cg09529433 | 220 | 22.00% | chr17 | 48637255 | + | chr17: 48636103-48639279 | Island | CACNA1G |
| cg10096645 | 220 | 22.00% | chr18 | 24130851 | + | chr18: 24126780-24131138 | Island | KCTD1 |
| cg06108383 | 211 | 21.10% | chr6 | 32120899 | − | chr6: 32121829-32122529 | N_Shore | PPT2; PRRT1 |
| cg03884082 | 172 | 17.20% | chr1 | 19971709 | + | chr1: 19970255-19971923 | Island | NBL1 |
| cg01065003 | 171 | 17.10% | chr18 | 24130839 | − | chr18: 24126780-24131138 | Island | KCTD1 |
| cg22647713 | 168 | 16.80% | chr10 | 8095697 | − | chr10: 8091374-8098329 | Island | FLJ45983; GATA3 |
| cg20449692 | 162 | 16.20% | chr3 | 170136920 | − | chr3: 170136242-170137886 | Island | CLDN11 |
| cg07136023 | 150 | 15.00% | chr16 | 86537316 | − | chr16: 86539118-86539486 | N_Shore | |
| cg20811659 | 136 | 13.60% | chr17 | 48637730 | − | chr17: 48636103-48639279 | Island | CACNA1G |
| cg20048434 | 132 | 13.20% | chr10 | 116163160 | − | chr10: 116163391-116164599 | N_Shore | AFAP1L2 |
| cg06546607 | 127 | 12.70% | chr19 | 34013019 | + | chr19: 34012271-34012936 | S_Shore | PEPD |
| cg00403498 | 127 | 12.70% | chr6 | 32119923 | − | chr6: 32121829-32122529 | N_Shore | PRRT1; PPT2 |
| cg20891301 | 119 | 11.90% | chr4 | 4864711 | − | chr4: 4864456-4864834 | Island | MSX1 |
| cg17416730 | 116 | 11.60% | chr6 | 33245541 | − | chr6: 33244677-33245554 | Island | B3GALT4 |
| cg01724566 | 113 | 11.30% | chr17 | 26926132 | + | chr17: 26925742-26926512 | Island | SPAG5 |
| cg16501308 | 112 | 11.20% | chr18 | 30350221 | − | chr18: 30349690-30352302 | Island | KLHL14 |
| cg06230736 | 109 | 10.90% | chr10 | 8096650 | + | chr10: 8091374-8098329 | Island | FLJ45983; GATA3 |
| cg03199651 | 105 | 10.50% | chr4 | 4862770 | − | chr4: 4864456-4864834 | N_Shore | MSX1 |
| cg06329022 | 103 | 10.30% | chr17 | 26926511 | + | chr17: 26925742-26926512 | Island | SPAG5 |
| cg13879776 | 102 | 10.20% | chr3 | 170136263 | − | chr3: 170136242-170137886 | Island | CLDN11 |

1. A method for detecting CpG methylation, comprising the steps of:
obtaining DNA from a biological sample obtained from a kidney allograft;
detecting methylation on a set of CpGs in the DNA of the sample;
wherein the set of CpGs is comprising at least 4 CpGs selected from the group consisting of Set A; at least 4 CpGs selected from the group consisting of Set B; or at least 4 CpGs selected from the group consisting Set A and Set B;
wherein Set A is cg06720949, cg17271223, cg19044229, cg00061520, cg01900755, cg11782729, cg16883450, cg26096304, cg02665578, cg02422197, cg23083046, cg05726208, cg19610659, cg08606493, cg09195780, cg10288719, cg11903872, cg12471836, cg20626616, cg25273619, cg26407571, cg09202851, cg14097773, cg14497910, cg22960616, cg01649611, cg09589331, cg10255171, cg14982576, cg21541534, cg23931819, cg24332389, cg24508633, cg03929366, cg11381106, cg15662465, cg00910503, cg07949722, cg18982976, cg03544320, cg04860664, cg08118957, cg13573626, cg15567016, cg16695176, cg00320453, cg00767269, cg02404377, cg02589501, and cg04644353;
wherein Set B is cg18714712, cg23872081, cg00449941, cg00505001, cg00765922, cg01102477, cg01608635, cg01724566, cg01863682, cg01885291, cg01912015, cg02077276, cg02445909, cg02648847, cg02885694, cg03656020, cg04279973, cg04603730, cg04751133, cg04801617, cg04948892, cg04962528, cg05214390, cg05951603, cg06329022, cg06774283, cg07063068, cg07065803, cg07096772, cg07274618, cg07298257, cg07563569, cg07647164, cg08332990, cg08696866, cg08812189, cg09620840, cg10239194, cg10305311, cg10500512, cg10927449, cg10992014, cg11178170, cg11471138, cg12064947, cg12402251, cg12534549, cg13156931, cg13273128, and cg13349607.

2. The method according to claim 1 wherein the set of CpGs is selected from Set A, and wherein the kidney allograft is at risk of developing glomerulosclerosis.

3. The method according to claim 1 wherein the set of CpGs is selected from Set B, and wherein the kidney allograft is at risk of developing interstitial fibrosis.

4. The method according to claim 1, further comprising detecting, in the DNA of the sample, methylation on a CpG of a CpG island selected from the group consisting of Set C; on a CpG selected from the group consisting of Set D; or on a CpG selected from the group consisting of Set E;
wherein Set C is chr1:152008838-152009112, chr1:156877769-156878649, chr1:16085147-16085862, chr1:19970255-19971923, chr1:32169537-32169869, chr2:27579296-27580135, chr2: 66672431-66673636, chr2:74781494-74782685, chr2:85640969-85641259, chr2:85980499-85982198, chr3:128205495-128212274, chr3:146187108-146187710, chr3:170136242-170137886, chr3:44802852-44803618, chr4:4864456-4864834, chr4: 79472806-79473177, chr5:150051116-150052107, chr6:10882926-10883149, chr6:30852102-30852676, chr6:32121829-32122529, chr6:33244677-33245554, chr6:37503538-37504291, chr6:44187186-44187400, chr6:56818873-56820308, chr7:120969587-120970743, chr7:27190274-27191115, chr7:63505977-63506298, chr8:41165852-41167140, chr9:1050078-1050510, chr10: 116163391-116164599, chr10:8091374-8098329, chr11:119186947-119187894, chr11:65325081-65326209, chr11:79148358-79152200, chr11:94706291-94707060, chr12:49738680-49740841, chr12:57609976-57611168, chr13:50697984-50702286, chr14:61746804-61748141, chr14:61787880-61789467, chr15:101389732-101390260, chr15:41217789-41223180, chr15:71407656-71408498, chr15:72522131-72524238, chr15:74218696-74220373, chr16:66958733-66959655, chr16:68298012-68298979, chr16: 86539118-86539486, chr17:14204168-14207702, chr17:1952919-1962328, chr17:26925742-26926512, chr17:48585385-48586167, chr17:48636103-48639279, chr17: 74706465-74707067, chr18:24126780-24131138, chr18:30349690-30352302, chr19: 1465206-1471241, chr19:34012271-34012936, chr19:46916587-46916862, chr19:47922251-47922777, chr19:496158-496481, chr19:50931270-50931638, chr20:37230523-37230742, chr21:34395128-34400245, chr21:46785130-46785339, and chr22:32339933-32341192;
wherein Set D is cg01811187, cg17078427, cg16547027, cg19596468, cg14309111, cg17603502, cg08133931, cg18599069, cg24840099, cg09529433, cg10096645, cg06108383, cg03884082, cg01065003, cg22647713, cg20449692, cg07136023, cg20811659, cg20048434, cg06546607, cg00403498, cg20891301, cg17416730, cg01724566, cg16501308, cg06230736, cg03199651, cg06329022, cg13879776, cg09024124, cg01507046, cg17113856, cg07846167, cg18701660, cg07516470, cg21096399, cg18187680, cg16519300, cg06375949, cg22590761, cg26292521, cg00110832, cg04255616, cg27426707, cg24605046, cg12883279, cg18454685, cg25426302, cg16650717, cg26270195, cg00449941, cg23058185, cg03970849, cg09998861, cg19315863, cg17960080, cg12163955, cg06206801, cg06803850, cg10049535, cg14098681, cg20652404, cg08238215, cg13934406, cg25144207, cg25755953, cg24329557, cg00319655, cg03189210, cg04963480, cg04262471, cg17182507, cg02048416, cg07346931, cg20328456, cg06023994, cg07434518, cg11590420, cg14176930, cg15520477, cg04749507, cg08062469, cg12741994, cg19679989, cg20663200, cg23943136, cg13398291, cg14315444, cg23520930, cg03682712, cg22880620, cg25987744, cg26381352, cg02551743, cg11522683, cg02989257, cg08707112, cg14327531, cg23359665, cg00868875, cg21785145, cg11129609, cg17566118, cg02241055, cg05942574, cg10074727, cg01803928, cg05671070, cg12064947, cg12730771, cg24509300, cg00086577, cg11386011, cg01111041, cg04164190, cg07841173, cg19657198, cg20155566, cg23104954, cg02344539, cg11731114, cg03696345, cg04186868, cg07060913, cg09573795, cg19882268, cg20654074, cg02503117, cg08076158, cg12626589, cg13484546, cg14261472, cg14294793, cg15330117, cg17991695, cg02694099, cg11071401, cg15472071, cg08306084, cg13882090, cg16662821, cg19814946, cg01546243, cg01626459, cg04216597, cg07147364, cg11303127, cg11950383, cg16481280, cg19333963, cg21333861, cg04641787, cg05620923, cg06018514, cg06133205, cg09255732, cg09337254, cg14040722, cg15187550, cg16553500, cg18923740, cg20682981, cg21249595, cg27390596, cg02962630, cg10169241, cg12103626, cg18932158, cg19450714, cg01070078, cg06774283, cg06814287, cg11145160, cg14130039, cg19036075, cg21538208, cg22314314, cg22322679, cg23010452, cg23047693, cg00316759, cg04209727, cg04856022, cg04877280, cg05945782, cg26579986, cg26704078, cg27147350, cg03740978, cg03839949, cg04982951, cg05133205, cg08347183, cg10551329, cg16226644, cg20281962, cg20914572, cg26366048, cg01312445, cg01993576, cg03995156, cg07555797, cg09942293, cg10372921, cg11941520, cg16396284, cg16710894, cg20161179, cg24092179, cg00552704, cg05176991, cg06902929, cg07273125, cg08483834, cg08510658, cg08890824, cg10094078, cg11215918, cg14167596, cg15852223, cg17639046, cg19951298, cg20196291, cg21973370, cg22648949, cg26784201, cg00360474, cg00930833, cg01149449, cg02388150, cg03718845, cg03832440, cg04414274, cg06870728, cg07132710, cg07306737, cg09857513, cg11014463, cg11626629, cg12599673, cg14293300, cg14904908, cg15140798, cg15839448, cg17124583, cg17764989, cg19156220, cg22216643, cg23599559, cg24858591, cg01160692, cg01271812, cg01626899, cg01684248, cg02980693, cg03306486, cg06022942, cg06747432, cg06844968, cg08438366, cg09042577, cg09748975, cg10464312, cg10633838, cg13438549, cg15355859, cg15709766, cg17029019, cg17891011, cg18774642, cg19241689, cg20706438, cg21068480, cg25520679, cg26055446, cg00040007, cg00927777, cg01616215, cg01725608, cg01785568, cg01796075, cg02956248, cg03814826, cg04203646, cg04751149, cg05003322, cg05871997, cg06025456, cg06283368, cg12881557, cg14250833, cg14914519, cg16838838, cg16868298, cg17276021, cg17372269, cg18374181, cg18729787, cg19884965, cg20138264, cg20152539, cg20180247, cg20283670, cg21435190, cg23253569, cg24399924, cg24888989, cg25075776, cg26418770, cg26657382, cg26977644, cg00183916, cg00313401, cg00592510, cg00702638, cg00739593, cg00913604, cg01404873, cg01807770, cg02151609, cg02242344, cg02339682, cg02429905, cg02836487, cg03133371, cg03270204, cg03356734, cg03365354, cg03434432, cg03570994, cg03575666, cg04105091, cg04436755, cg04852949, cg04983516, cg05457563, cg05470554, cg05713782, cg05946971, cg06065141, cg06485671, cg06515159, cg06642647, cg06892009, cg07137845, cg07265873, cg07348922, cg07578663, cg08110052, cg08509237, cg08711175, cg09074260, cg09172659, cg09410389, cg09535924, cg09570958, cg09673208, cg09829319, cg10405604, cg10541674, cg10935762, cg10948797, cg11018337, cg11452354, cg11453400, cg11471939, cg12280317, cg12308216, cg13102294, cg13161961, cg13333304, cg13365340, cg13431023, cg13524919, cg13543854, cg13793145, cg13915354, cg13951527, cg14435807, cg14448169, cg14775296, cg14843922, cg14950855, cg15543281, cg15657704, cg15848031, cg15989091, cg16004427, cg16079541, cg16437908, cg16477774, cg16713743, cg18729886, cg19873719, cg20457147, cg20459712, cg20731875, cg21415424, cg22609784, cg22745102, cg22913903, cg22931738, cg23305408, cg23519308, cg23621097, cg23950233, cg24506025, cg25161092, cg25484790, cg26709950, cg27038439, cg27070869, and cg27357571; and
wherein Set E is cg01811187, cg17078427, cg16547027, cg19596468, cg14309111, cg17603502, cg08133931, cg18599069, cg24840099, cg09529433, cg10096645, cg06108383, cg03884082, cg01065003, cg22647713, cg20449692, cg07136023, cg20811659, cg20048434, cg06546607, cg00403498, cg20891301, cg17416730, cg01724566, cg16501308, cg06230736, cg03199651, cg06329022, and cg13879776.

5. The method according to claim 1, further comprising detecting, in the DNA of the sample, methylation on a set of at least 4 CpGs chosen from Table 7 selected from Set E.

6. A method for predicting the risk of developing chronic kidney allograft injury, comprising the steps of:
obtaining DNA from a biological sample obtained from the;
detecting methylation on a set of CpGs in the DNA of the sample;
predicting the allograft to be at risk of developing chronic injury when the methylation detected on the set of CpGs is higher compared to reference values of methylation on the same set of CpGs;
wherein the set of CpGs comprises:
at least 1 CpG chosen from Set A, or at least 1 CpG chosen from Set B; and
at least 1 CpG chosen from Set C, at least 1 CpG chosen from Set D, or at least 1 CpG chosen Set E; and
wherein the set of CpGs comprises at least 4 CpGs chosen from the combination of the CpGs in Sets A-E;
wherein Set A is cg06720949, cg17271223, cg19044229, cg00061520, cg01900755, cg11782729, cg16883450, cg26096304, cg02665578, cg02422197, cg23083046, cg05726208, cg19610659, cg08606493, cg09195780, cg10288719, cg11903872, cg12471836, cg20626616, cg25273619, cg26407571, cg09202851, cg14097773, cg14497910, cg22960616, cg01649611, cg09589331, cg10255171, cg14982576, cg21541534, cg23931819, cg24332389, cg24508633, cg03929366, cg11381106, cg15662465, cg00910503, cg07949722, cg18982976, cg03544320, cg04860664, cg08118957, cg13573626, cg15567016, cg16695176, cg00320453, cg00767269, cg02404377, cg02589501, and cg04644353;
wherein Set B is cg18714712, cg23872081, cg00449941, cg00505001, cg00765922, cg01102477, cg01608635, cg01724566, cg01863682, cg01885291, cg01912015, cg02077276, cg02445909, cg02648847, cg02885694, cg03656020, cg04279973, cg04603730, cg04751133, cg04801617, cg04948892, cg04962528, cg05214390, cg05951603, cg06329022, cg06774283, cg07063068, cg07065803, cg07096772, cg07274618, cg07298257, cg07563569, cg07647164, cg08332990, cg08696866, cg08812189, cg09620840, cg10239194, cg10305311, cg10500512, cg10927449, cg10992014, cg11178170, cg11471138, cg12064947, cg12402251, cg12534549, cg13156931, cg13273128, and cg13349607;
wherein Set C is chr1: 152008838-152009112, chr1: 156877769-156878649, chr1: 16085147-16085862, chr1: 19970255-19971923, chr1:32169537-32169869, chr2:27579296-27580135, chr2:66672431-66673636, chr2:74781494-74782685, chr2:85640969-85641259, chr2:85980499-85982198, chr3:128205495-128212274, chr3:146187108-146187710, chr3:170136242-170137886, chr3:44802852-44803618, chr4:4864456-4864834, chr4:79472806-79473177, chr5:150051116-150052107, chr6:10882926-10883149, chr6:30852102-30852676, chr6:32121829-32122529, chr6:33244677-33245554, chr6:37503538-37504291, chr6:44187186-44187400, chr6:56818873-56820308, chr7:120969587-120970743, chr7:27190274-27191115, chr7:63505977-63506298, chr8:41165852-41167140, chr9:1050078-1050510, chr10: 116163391-116164599, chr10:8091374-8098329, chr11:119186947-119187894, chr11:65325081-65326209, chr11:79148358-79152200, chr11:94706291-94707060, chr12:49738680-49740841, chr12:57609976-57611168, chr13:50697984-50702286, chr14:61746804-61748141, chr14:61787880-61789467, chr15:101389732-101390260, chr15:41217789-41223180, chr15:71407656-71408498, chr15:72522131-72524238, chr15:74218696-74220373, chr16:66958733-66959655, chr16:68298012-68298979, chr16: 86539118-86539486, chr17:14204168-14207702, chr17:1952919-1962328, chr17:26925742-26926512, chr17:48585385-48586167, chr17:48636103-48639279, chr17: 74706465-74707067, chr18:24126780-24131138, chr18:30349690-30352302, chr19: 1465206-1471241, chr19:34012271-34012936, chr19:46916587-46916862, chr19:47922251-47922777, chr19:496158-496481, chr19:50931270-50931638, chr20:37230523-37230742, chr21:34395128-34400245, chr21:46785130-46785339, and chr22:32339933-32341192;
wherein Set D is cg01811187, cg17078427, cg16547027, cg19596468, cg14309111, cg17603502, cg08133931, cg18599069, cg24840099, cg09529433, cg10096645, cg06108383, cg03884082, cg01065003, cg22647713, cg20449692, cg07136023, cg20811659, cg20048434, cg06546607, cg00403498, cg20891301, cg17416730, cg01724566, cg16501308, cg06230736, cg03199651, cg06329022, cg13879776, cg09024124, cg01507046, cg17113856, cg07846167, cg18701660, cg07516470, cg21096399, cg18187680, cg16519300, cg06375949, cg22590761, cg26292521, cg00110832, cg04255616, cg27426707, cg24605046, cg12883279, cg18454685, cg25426302, cg16650717, cg26270195, cg00449941, cg23058185, cg03970849, cg09998861, cg19315863, cg17960080, cg12163955, cg06206801, cg06803850, cg10049535, cg14098681, cg20652404, cg08238215, cg13934406, cg25144207, cg25755953, cg24329557, cg00319655, cg03189210, cg04963480, cg04262471, cg17182507, cg02048416, cg07346931, cg20328456, cg06023994, cg07434518, cg11590420, cg14176930, cg15520477, cg04749507, cg08062469, cg12741994, cg19679989, cg20663200, cg23943136, cg13398291, cg14315444, cg23520930, cg03682712, cg22880620, cg25987744, cg26381352, cg02551743, cg11522683, cg02989257, cg08707112, cg14327531, cg23359665, cg00868875, cg21785145, cg11129609, cg17566118, cg02241055, cg05942574, cg10074727, cg01803928, cg05671070, cg12064947, cg12730771, cg24509300, cg00086577, cg11386011, cg01111041, cg04164190, cg07841173, cg19657198, cg20155566, cg23104954, cg02344539, cg11731114, cg03696345, cg04186868, cg07060913, cg09573795, cg19882268, cg20654074, cg02503117, cg08076158, cg12626589, cg13484546, cg14261472, cg14294793, cg15330117, cg17991695, cg02694099, cg11071401, cg15472071, cg08306084, cg13882090, cg16662821, cg19814946, cg01546243, cg01626459, cg04216597, cg07147364, cg11303127, cg11950383, cg16481280, cg19333963, cg21333861, cg04641787, cg05620923, cg06018514, cg06133205, cg09255732, cg09337254, cg14040722, cg15187550, cg16553500, cg18923740, cg20682981, cg21249595, cg27390596, cg02962630, cg10169241, cg12103626, cg18932158, cg19450714, cg01070078, cg06774283, cg06814287, cg11145160, cg14130039, cg19036075, cg21538208, cg22314314, cg22322679, cg23010452, cg23047693, cg00316759, cg04209727, cg04856022, cg04877280, cg05945782, cg26579986, cg26704078, cg27147350, cg03740978, cg03839949, cg04982951, cg05133205, cg08347183, cg10551329, cg16226644, cg20281962, cg20914572, cg26366048, cg01312445, cg01993576, cg03995156, cg07555797, cg09942293, cg10372921, cg11941520, cg16396284, cg16710894, cg20161179, cg24092179, cg00552704, cg05176991, cg06902929, cg07273125, cg08483834, cg08510658, cg08890824, cg10094078, cg11215918, cg14167596, cg15852223, cg17639046, cg19951298, cg20196291, cg21973370, cg22648949, cg26784201, cg00360474, cg00930833, cg01149449, cg02388150, cg03718845, cg03832440, cg04414274, cg06870728, cg07132710, cg07306737, cg09857513, cg11014463, cg11626629, cg12599673, cg14293300, cg14904908, cg15140798, cg15839448, cg17124583, cg17764989, cg19156220, cg22216643, cg23599559, cg24858591, cg01160692, cg01271812, cg01626899, cg01684248, cg02980693, cg03306486, cg06022942, cg06747432, cg06844968, cg08438366, cg09042577, cg09748975, cg10464312, cg10633838, cg13438549, cg15355859, cg15709766, cg17029019, cg17891011, cg18774642, cg19241689, cg20706438, cg21068480, cg25520679, cg26055446, cg00040007, cg00927777, cg01616215, cg01725608, cg01785568, cg01796075, cg02956248, cg03814826, cg04203646, cg04751149, cg05003322, cg05871997, cg06025456, cg06283368, cg12881557, cg14250833, cg14914519, cg16838838, cg16868298, cg17276021, cg17372269, cg18374181, cg18729787, cg19884965, cg20138264, cg20152539, cg20180247, cg20283670, cg21435190, cg23253569, cg24399924, cg24888989, cg25075776, cg26418770, cg26657382, cg26977644, cg00183916, cg00313401, cg00592510, cg00702638, cg00739593, cg00913604, cg01404873, cg01807770, cg02151609, cg02242344, cg02339682, cg02429905, cg02836487, cg03133371, cg03270204, cg03356734, cg03365354, cg03434432, cg03570994, cg03575666, cg04105091, cg04436755, cg04852949, cg04983516, cg05457563, cg05470554, cg05713782, cg05946971, cg06065141, cg06485671, cg06515159, cg06642647, cg06892009, cg07137845, cg07265873, cg07348922, cg07578663, cg08110052, cg08509237, cg08711175, cg09074260, cg09172659, cg09410389, cg09535924, cg09570958, cg09673208, cg09829319, cg10405604, cg10541674, cg10935762, cg10948797, cg11018337, cg11452354, cg11453400, cg11471939, cg12280317, cg12308216, cg13102294, cg13161961, cg13333304, cg13365340, cg13431023, cg13524919, cg13543854, cg13793145, cg13915354, cg13951527, cg14435807, cg14448169, cg14775296, cg14843922, cg14950855, cg15543281, cg15657704, cg15848031, cg15989091, cg16004427, cg16079541, cg16437908, cg16477774, cg16713743, cg18729886, cg19873719, cg20457147, cg20459712, cg20731875, cg21415424, cg22609784, cg22745102, cg22913903, cg22931738, cg23305408, cg23519308, cg23621097, cg23950233, cg24506025, cg25161092, cg25484790, cg26709950, cg27038439, cg27070869, and cg27357571; and
wherein Set E is cg01811187, cg17078427, cg16547027, cg19596468, cg14309111, cg17603502, cg08133931, cg18599069, cg24840099, cg09529433, cg10096645, cg06108383, cg03884082, cg01065003, cg22647713, cg20449692, cg07136023, cg20811659, cg20048434, cg06546607, cg00403498, cg20891301, cg17416730, cg01724566, cg16501308, cg06230736, cg03199651, cg06329022, and cg13879776.

7. The method according to claim 1, wherein the biological sample is taken at the time of implantation.

8. The method of according to claim 1, wherein said biological sample is a biopsy sample from an allograft.

9. The method of according to claim 1, wherein said biological sample is a liquid biopsy sample.

10. The method according to claim 1, further comprising administering toe the recipient an inhibitor of hypermethylation, a demethylating agent, or an inhibitor of fibrosis.

11. The method according to claim 10, wherein the inhibitor of hypermethylation is a stimulator of TET enzyme.

12. The method according to claim 11, wherein said stimulator of TET enzyme is an inhibitor of the BCAT1 enzyme.

13. The method according to claim 10, wherein the inhibitor of fibrosis is a demethylating agent or a Jnk-inhibitor.

14. (canceled)

15. (canceled)

16. (canceled)

17. (canceled)

18. The method according to claim 1, wherein the biological sample is taken up to 3 months post-implantation.

19. The method according to claim 6, wherein the biological sample is taken at the time of implantation.

20. The method according to claim 6, wherein the biological sample is taken up to 3 months post-implantation.

21. The method according to claim 6, wherein said biological sample is a biopsy sample from an allograft.

22. The method according to claim 6, wherein said biological sample is a liquid biopsy sample.

23. The method according to claim 6, further comprising administering toe the recipient an inhibitor of hypermethylation, a demethylating agent, or an inhibitor of fibrosis.

24. The method according to claim 23, wherein the inhibitor of hypermethylation is a stimulator of TET enzyme.
